SEQUENCE LISTING (140 sequences)

SEQ ID NO: 1 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 2 - protein, 503 aa, Brassica juncea
MAILDSGGVAVPPTENGVADLDRLHRRKSRSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRPIIENLMKYGWLIRTDFWFSSTSLRDWPLFMCCLSLSVFPLAAFTVEKMVLQKFISEPVAIILHVIITLTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQFAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFWFSFCIFGQPMCVLLYYHDLMNRKGSMS

SEQ ID NO: 3 - protein, 503 aa, Brassica napus
MAILDSGGVAVPPTENGVADLDRLHRRKSSSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRLIIENLMKYGWLIRTDFWFSSTSLRDWPLFMCCLSLSVFPLAAFTVEKMVLQKFISEPVAIILHVIITMTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQLAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFGSASCIFGQPMCGLLYYHDLMNRKGSMS

SEQ ID NO: 4 - protein, 503 aa, Brassica juncea
MAILDSGTVTMATENGVADLDMLRRRKSRSDSSNGLLSETSPSDDAGAPADVEDRVDSAAQGTANLAGDTETRESGGGGGGGNGEVRFTYRPSVPAHRRTRESPLSSDAIFKQSHAGLFNLCVVVLVAVNSRLIIENLMKYGWLIRTDFWFSSTSLRDWPLFMCCLSLSIFPLAAFTVEKLVLQKFISEPVVIILHIIITMTEVLYPVYVTLRCDSAFLSGVTLMLLTCIVWLKLVSYAHTNYDIRTVANSADKVDPEVSYYVSLKSLAYFMVAPTLCYQPSYPRSPGIRKGWVARQFAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFIGIMFQVPLVFITNYLQERFGSMVGNMIFWFSFCIFGQPMCVLLYYHDLMNRKGSMS

SEQ ID NO: 5 - protein, 518 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEK

SEQ ID NO: 6 - protein, 473 aa, Echium pitardii
MTIWESPEIISSDEAAAALRRRGGAKEVAEQRLDSEEEKKKEEENGKLKYTYRASAPAHRRIKESPLSSDAIFKQSHAGLFNLCIVVLVAVNSRLIIENLMKYGWLISSGFWLSSTSLSDWPLLICCLSLPIFPLASFVVEKLSQQEFISEQVVITLHALITTTVIMYPVIVILRCDPAVLSGVILMLFTCIVWLKLVSYAHTNYDMRALAKDCDKLQALSGSSMEDCSFEVNFQALVYFMVAPTLCYQLRYPRTPCIRWGWVTRHLIKLIIFTGLMGFIIEQYINPIVKNSQHPLKGNLLYAIERVLKLSVPNIYVWLCMFYCFFHLWLNILAELLCFGDREFYKDWWNAQTIEEYWRMWNMPVHKWMVRHIYFPCLRNGMPKELAILIAFLISAIFHELCIAVPCHIFKFWAFIGIMFQVPLVLLTNVLVKKFQNSMVGNMIFWCFFCILGQPMSLLLYYHDVLNRKVNAN

SEQ ID NO: 7 - protein, 523 aa, Vernonia galamensis
MALLDTPQIGEITTTATTTIRRRTTVKPDAGIGDGLFDSSSSSKTNSSFEDGDSLNGDFNDKFKEQIGAGDESKDDSKGNGQKIDHGGVKKGRETTVVHYAYRPSSPAHRRIKESPLSSDAIFKQSHAGLFNLCIVVLVAVNGRLIIENLMKYGLLINSNFWFSSRSLRDWPLLMCCLTPSDFPLAAYIVEKLAWKKRISDPVVITLHVIITTTAILYPVFMILRFDSVVLSGVSLMLCACINWLKLVSFVHTNYDMRSLLNSTDKGEVEPMSSNMDYFYDVNFKSLVYFMVAPTLCYQISYPRTAFIRKGWVLRQLIKLVIFTGFMGFIIEQYINPIVKNSRHPLKGDFLYAIERVLKLSVPNLYVWLCMFYCFFHLWLNILAELLCFGDREFYKDWWNAQTIEEYWRLWNMPVHKWIVRHLYFPCLRNGIPKGAAILVAFFMSAVFHELCIAVPCHIFKFWAFIGIMFQVPLVLLTNYLQHKFQNSMVGNMIFWCFFSIFGQPMCVLLYYHDVMNQKGKSK

SEQ ID NO: 8 - protein, 532 aa, Nicotiana tabacum
MVIMELPESVEMTTTTTTSGIENLNSDLNHSVRRRRGSNGFEAASAINSSDANMSEDRRDVCGSGAGLETVNERSKSVGESSDVIRKEDDRNDNVANGEESKSTETTTTPFKFAYRASAPAHRRIKESPLSSDAIFKQSHAGLFNLCVVVLIAVNSRLIIENLMKYGLLIRAGFWFSSKSLRDWPLLMCCLSLQILPLAAFLVEKLAQQRHLTERAVVTLHITITTAAILYPVLVILGCDSAFLFGVILMLVACIVWMKLVSYAHTNHDMRQLAKSTDKDETSDGDFSYDVSFKSLAYFMVAPTLCYQLSYPHTPCIRKGWVARQFIKLVIFTGLMGFIIEQYINPIVQNSQHPLKGNLLYAIERVLKLSVPNLYVWLCMFYCFFHLWLNILAELLCFGDREFYKDWWNAKTIDEYWRMWNMPVHKWMVRHIYFPCLRNGIPKGVAILIAFLVSAVFHELCIAVPCRLFKWWAFMGIMFQVPLVILTNFLQNKFQSSMVGNMMFWCFFCILGQPMCVLLYYHDVMNRKSSAR

SEQ ID NO: 9 - protein, 534 aa, Perilla frutescens
MAILDSPEILDTTSSSADNGAAHHTTLRRRQSARSVPPLLDSDSNSLEAESAINDSENVRNDANLIENLRGGAVESENEKQESYGKEEGAKVKENGETSNGNGTDVMAVKFTFRPAAPAHRKNKESPLSSDAIFKQSHAGLFNLCIVVLVAVNSRLIIENLMKYGWLIKSGFWFSSTSLRDWPLLMCCLSLPVFALASFLVEKLVKLNYIPEWVAVFLHVTITTVEILFPVVVILRCDSAVLSGVTLMLFACTVWLKLVSYAHTNYDLRVLAKSLDKWEAMSRYWNLDYAYDVSFKSLAYFMVAPTLCYQPSYPRTACIRKGWVVRQLIKLVIFTGLMGFIIEQYINPIVQNSQHPLKGNLLYAIERVLKLSVPNLYVWLCMFYCFFHLWLNILAELLCFGDREFYKDWWNARTVEEYWRMWNMPVHKWMVRHIYCPCLQNGIPKIVAVLIAFLVSAIFHELCVAVPCQIFKFWAFSGIMLQVPLVIVTNYLQEKFKNSMVGNMMFWCFFCIFGQPMCVLLYYHDLMNRKASAR

SEQ ID NO: 10 - protein, 512 aa, Zea mays
MADSEDAPPAVHRRPPRPARGAAAAQGFAAALRRRLRSGAAVAARASFAADSGDESGPGEPSSSRRRDNSGGASSAAGGRAGAGDFSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAK

SEQ ID NO: 11 - protein, 515 aa, Sorghum bicolor
MADTDDAPPAPAVHRRPPRPARGAAAAQGFAAKLRRRLSSGAAAAARASFAADSGDESGPGEPSSSRRRDNGGDASSAADGGRGGAGDFSAFTFRAAAPVHRKAKESPLSSDAIFKQSHAGLFNLCIVVLVAVNSRLIIENLMKYGLLIRSGFWFNATSLRDWPLLMCCLSLPVFPLGAFAVEKLAFNNLITDAAATCFHIFLTTLEIVYPVLVILKCDSAVLSGFVLMFIACIVWLKLVSFAHTNHDIRKLITSGKKVDNELTVADIDNLQAPTLGSLTYFMMAPTLCYQPSYPRTPYVRKGWLVRQVILYLIFTGLQGFIIEQYINPIVVNSQHPLKGGLLNAVETVLKLSLPNVYLWLCMFYCLFHLWLNILAEILRFGDREFYKDWWNAKTIDEYWRKWNMPVHKWMLRHIYFPCIRNGISKEVAAFIAFFVSAVFHELCVAVPCHILKFWAFLGIMLQIPLIILTSYLKNKFNDTMVGNMIFWFFFCIYGQPMCVLLYYHDVMNRTEKTK

SEQ ID NO: 12 - protein, 538 aa, Oryza sativa
MVGSDGDGDGGGGEAHAPAAPAHHHRRPPRPRGGSGAIVEGFAAALRRRIRSGAAAAARASFGGDSGDEAASGEPSSSSSSSPSRRRGGDSNGAEASSAAGGGGGRGGGGDFSAFTFRAAAPVHRKAKESPLSSDAIFKQSHAGLFNLCIVVLVAVNSRLIIENLMKYGLLIRAGFWFNDKSLRDWPLLMCCLSLPAFPLGAFAVEKLAFNNVITDAVATCLHIFLSTTEIVYPVLVILKCDSAVLSGFLLIFIACIVWLKLVSFAHTNHDIRQLTMGGKKVDNELSTVDMDNLQPPTLGNLIYFMMAPTLCYQPSYPRTSCVRKGWLIRQIILYLIFTGLQGFIIEQYINPIVVNSQHPLKGGLLNAVETVLKLSLPNVYLWLCMFYAFFHLWLSILAEILRFGDREFYKDWWNAKTIDEYWRKWNMPVHKWVVRHIYFPCMRNGISKEVAVLISFLVSAVLHEICVAVPCRILKFWAFLGIMLQIPLIVLTAYLKSKFRDTMVGNMIFWFFFCIYGQPMCLLLYYHDVMNRIEKAR

SEQ ID NO: 13 - protein, 477 aa, Oryza sativa
MAPPPSLAPDRGGGEPDDALRLRARAAAAAGDAPAPQQQQEQRHQEQQQQLLWYRASAPAHRRVRESPLSSDAIFRQSHAGLLNLCIVVLVAVNSRLIIENLMKYGLLIRAGFWFSGTSLADWPLLMCCLTLPTFPLAALMVEKLAQRKLISKHVVILLHIVITTSVLVYPVVVILKCDSAVLSGFVLMFLASIIWLKLVSFAHTNYDIRMLSKSIEKGVTHDISIDPENIKWPTFKRLSYFMLAPTLCYQPSYPRTTYIRKGWVVRQLIKCLVFTGLMGFIIEQYINPIVKNSKHPLKGNFLNAIERVLKLSVPTLYVWLCMFYCFFHLWLNILAELLCFGDREFYKDWWNAKTVEEYWRMWNMPVHKWVIRHIYFPCIRNGFSKGVAILISFLVSAAFHELCVAVPCHIFKFWAFIGIMFQIPLVFLTKYLQDKFNNTMVGNMIFWFFFSILGQPMCVLLYYHDVMNRQQAQTNR

SEQ ID NO: 14 - protein, 529 aa, Sorghum bicolor
MAPPPSMAAASDRAVPGADATEASSLRLRRAPSADAGDLADDSSGDRRENGEPQPPQEQQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLIRRKLIGEHVVILLHIIITTSVIVYPVVVTLKCDSAVLSGFLLMFLASIMWMKLVSYAHTNYDIRALSKSTEKGAAYGNYVDPESMKDPTFKSLVYFMLAPTLCYQPTYPRTTCIRKGWVTRQLIKCLVFTGLMGFIIEQYINPIVKNSKHPLKGNFLNAIERVLKLSVPTLYVWLCMFYCFFHLWLNILAELLCFGDREFYKDWWNAKTVEEYWRMWNMPVHKWIIRHIYFPCIRKGFSRGVAILVSFLVSAVFHEICIAVPCHIFKFWAFSGIMFQIPLVFLTRYLQATFKNIMVGNMIFWFFFSIVGQPMCVLLYYHDVMNRQAQQVDNSAETCTLRQVIRSRLERRSRKQQQQQASSPPLPLLPAS

SEQ ID NO: 15 - protein, 494 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASR

SEQ ID NO: 16 - protein, 447 aa, Physcomitrium patens
MPVKSSNLAGERAATSHINANTKFDLRGCTPAHRVRRESPLSSDAIFHQSHAGLFNLCIVVLIAVNSRLIIENLMKYGLLIRTGFWFSSKSARDWPLLMCGLSLPTFPFAALLVEKLCWKNENGKWLIFVLHLIISTVGILYPGYVIHRVQSALLPGLVLILIAVTGWMKLISYAHVNKDMRELLRAKEKLPEAPQYADKIEVPDHLTIQNIAYFMLAPTLCYQLSYPRSDTIRKSWVLRQAGKLVVFLGLGGFIIEQYINPTVKNSQHPLRGNYLQALERVLKLSLPVLYVWLCLFYCLFHLWLNIVAELLRFGDREFYKDWWNAQTVEEYWRMWNMPVHKWMVRHIYFPSIRAGLSKKAAVLLVFAISALFHEVIIGVPCHMLRCWAFLGIMMQVPLVYLTNVIKERYHSSMVGNMVFWFFFCIVGQPMCLLLYYHDVFNNFPST

SEQ ID NO: 17 - protein, 425 aa, Selaginella moellendorffii
MRPSLPAHRRSKESPLSSDAIFTQSHAGLFNLCIVVLVAVNSRLIIENLMKYGLLIQAEVLFSSKSLKDWPLLMCGLSLLIFPLAAYVIEKIKARRPATAVAPLHLINLAAALLYPIYVIEMFQSDLLSGLVLMLIAVTGWLKLVSYAHTNADIRAVKKDGGKIELPAEAPAIDYPDNISLKNIAYFMAAPTLCYQLSYPRSPRIRTGWVLRQLGKWIVFNGFMGFIIGQYMNPIIRNSTHPLKGNYLYAIERVLKLSIPTLYVWLGFFYCFFHLWLNIVAEILCFGDREFYKDWWNAKSVDEYWRLWNMPVHRWLVRHVYFPCLRLGLHKQFAILVVFVISGIFHEICIAVPCHMLRGWAFLGIMFQVPLVLVTNVLQRKFQSSMVGNMIFWFFFCIVGQPMCVLLYYHDVVNRQQLQLAGRSK

SEQ ID NO: 18 - protein, 507 aa, Euonymus alatus
MAANLNEASDLNFSLRRRTGGISSTTVPDSSSETSSSEADYLDGGKGAADVKDRGDGAVEFQNSMKNVERIEKHESRVGLDSRFTYRPSVPAHRTIKESPLSSDAIFKQSHAGLFNLCIVVLVAVNSRLIIENLMKYGWLIRSGFWFSSRSLRDWPLFMCCLTLPVFPLAAFLFEKLAQKNLISEPVVVLLHIVNTTAAVLYPVLVILRCDSAFMSGVTLMLFACIVWLKLVSYAHTNYDMRALTKSVEKGDTPLSSQNMDYSFDVNIKSLAYFMVAPTLCYQISYPRTPYVRKGWVVRQFVKLIIFTGLMGFIIEQYINPIVQNSQHPLKGNFLYAIERVLKLSVPNLYVWLCMFYCLFHLWLNILAELLCFGDREFYKDWWNAKTVEEYWRMWNMPVHKWMVRHIYFPCLRNGIPKGVAFVISFLVSAVFHELCIAVPCHIFKLWAFFGIMLQVPLVLITSYLQNKFRSSMVGNMMFWFSFCIFGQPMCLLLYYHDLMNRNGKME

SEQ ID NO: 19 - protein, 518 aa, Vitis vinifera
MAICNSPVSVTTSSSSSHADSDLDFSIRKRFGGKGKAVADSSLETETEAAAAAVLEAEKSVGEVGSGGDRGESGSQVVRNGENGVAEVAAKFAYRPCAPAHRKVKESPLSSDAIFRQSHAGLFNLCIVVLVAVNSRLIIENLMKYGWLIRAGFWFSSKSLRDWPLFMCCLTLPIFPLAAFVVEKLAQQKYISEQVVVSLHIIITTAAVLFPVLVILRCDSAVLSGVTLMLFACIVWLKLVSFAHTNYDMRAVAKLIDKGDDLSTSLNMDYPYDVNFKSLAYFMVAPTLCYQPSYPRSTCIRKGWVFRQFVKLAIFTGVMGFIIEQYINPIVQNSQHPLKGNFFYALERILKLSVPNLYVWLCMFYCFFHLWLNILAELLRFGDREFYKDWWNAKTVEEYWRMWNMPVHKWMVRHLYFPCLRNGISKGVSVVIAFAISAIFHELCIAVPCHMFKLWAFIGIMFQVPLVLVTNYLQNKFRNSMVGNMIFWLFFSILGQPMCVLLYYHDLMNRKETTESSL

SEQ ID NO: 20 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 21 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYYPCLRHGLPKAAALLIAFLVSALFHELCIAVPCHIFKLWAFGGIMFQVPLVLITNYLQNKFRNSMVGNMIFWFIFSILGQPMCVLLYYHDLMNRKGKLD

SEQ ID NO: 22 - protein, 511 aa, Lotus japonicus
MAISEDSESLFAAAAASSVIQSGSSVRRRPSAISAVATVEDESSSEEPVPVRDSGSDVDDSVSSEQHVSPATANREKNQVHDISATKFAYRPSAPAHRRVKESPLSSDNIFRHHAGLFNLCIVVLVAVNSRLIIENLMKYGWLIRTGFWFSSKSLRDWPLFMCCLSLAIFPFAAFVVEKLVQQKCISEPVVVLHIFISTAAVVYPVLVILRTDSAFPSGVTLMLFACIVWLKLVSYAHTNYDMRELTKSIEKGEALPNTLNMDYSYDVSFKSLAYFMIAPTLCYQPRYPRSPSIRKGWVLRQLVKLIIFTGVMGFIIEQYINPIVQNSQHPLKGNLLYAIERVLKLSVPNLYVWLCMFYCFFHLWLNILAELLRFGDREFYKDWWNAKTFEEYWRMWNMPVHKWMIRHLYFPCLRNGIPKGVAILIAFLVSALFHELCIAVPCHIFKLWAFGGIMFQVPLILITNYLQNKFRNSMVGNMIFWFIFSILGQPMAVLLYYHDLMNRKSKLDQS

SEQ ID NO: 23 - protein, 539 aa, Medicago truncatula
MAISDTPETTATATATVTTIETDTDLKRSSLRRRPSATSTAGGLFDAESAAADAVRDSGSDDSLNGKINNEEEVKDRKTDHAEGIVDDDDDNAVKKNGGNDVINDRENVAVDFKFTYRPSVPAHRRSKESPLSSGNIFRQSHAGLFNLCIVVLVAVNSRLIIENLMKYGWLIRSGFWFSSKSLRDWPLFMCCLSLAIFPLAAFVVEKLAQQKRISEPVIVLLHIVITTVAIIYPVLVILWCDSAFLSGSTLMLLTCIVWLKLVSYAHTTYDMRALAVSNEKGETMPDTFNMEEYPHNVSFQSLAYFMVAPTLCYQPSYPRTPSVRKGWVCRQLLKLVIFTGVMGFIIEQYMNPIVQNSQHPLKGNLLYAIERVLKLSVPNVYVWLCMFYCFFHLWLNILAELLRFGDREFYKDWWNAQTVEEYWRMWNMPVHKWMVRHVYFPCIRFGIPKGAAALTAFLVSAVFHELCIAVPCRMFKLWAFIGIMFQVPLVLITNYLKNKYRNSMVGNMIFWFIFCILGQPMCVLLYYHDLMNRKGEID

SEQ ID NO: 24 - protein, 521 aa, Jatropha curcas
MTILETTTSGGDGVAESSSDLNVSLRRRRKGTSSDGALPELTSNIVELESESGGQVMMDPGMVTEPETEKINGKDCGGDKDKIDNRENRGRSDIKFTYRPSVPAHRALRESPLSSDAIFKQSHAGLFNLCIVVLVAVNSRLIIENLMKYGWLIKTGFWFSSRSLRDWPLLMCCLTLPIFSLAAYLVEKLAYRKYISAPIVIFFHMLITTTAVLYPVSVILSCGSAVLSGVALMLFACIVWLKLVSYAHTNYDMRAIANSADKGDALSDTSGADSSRDVSFKSLVYFMVAPTLCYQPSYPRTDSVRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLIAFFVSAVFHELCIAVPCHMFKLWAFIGIMFQIPLVGITNYLQNKFRSSMVGNMIFWFIFCILGQPMCVLLYYHDLMNRKGNAELR

SEQ ID NO: 25 - protein, 526 aa, Vernicia fordii
MTIPETPDNSTDATTSGGAESSSDLNLSLRRRRTASNSDGAVAELASKIDELESDAGGGQVIKDPGAEMDSGTLKSNGKDCGTVKDRIENRENRGGSDVKFTYRPSVPAHRALKESPLSSDNIFKQSHAGLFNLCIVVLVAVNSRLIIENIMKYGWLIKTGFWFSSRSLRDWPLLMCCLTLPIFSLAAYLVEKLACRKYISAPTVVFLHILFSSTAVLYPVSVILSCESAVLSGVALMLFACIVWLKLVSYAHTNFDMRAIANSVDKGDALSNASSAESSHDVSFKSLVYFMVAPTLCYQPSYPRTASIRKGWVVRQFVKLIIFTGFMGFIIEQYINPIVQNSQHPLKGDLLYAIERVLKLSVPNLYVWLCMFYCFFHLWLNILAELLRFGDREFYKDWWNARTVEEYWRMWNMPVHKWMVRHIYFPCLRHKIPRGVALLITFFVSAVFHELCIAVPCHIFKLWAFIGIMFQIPLVGITNYLQNKFRSSMVGNMIFWFIFCILGQPMCLLLYYHDLMNRKGTTESR

SEQ ID NO: 26 - protein, 523 aa, Vernonia galamensis; identical to SEQ ID NO: 7

SEQ ID NO: 27 - protein, 521 aa, Ricinus communis
MTILETPETLGVISSSATSDLNLSLRRRRTSNDSDGALADLASKFDDDDDVRSEDSAENIIEDPVAAVTELATAKSNGKDCVANSNKDKIDSHGGSSDFKLAYRPSVPAHRSLKESPLSSDLIFKQSHAGLFNLCIVVLVAVNSRLIIENLMKYGWLIKTGFWFSSRSLRDWPLFMCCLSLPVFPLAAYLVEKAAYRKYISPPIVIFLHVIITSAAVLYPASVILSCESAFLSGVTLMELACMVWLKLVSYAHTNYDMRAIADTIHKEDASNSSSTEYCHDVSFKTLAYFMVAPTLCYQPSYPRTAFIRKGWVFRQFVKLIIFTGFMGFIIEQYINPIVQNSQHPLKGDLLYAIERVLKLSVPNLYVWLCLFYCFFHLWLNIVAELLRFGDREFYKDWWNAKTVEEYWRMWNMPVHKWMVRHIYFPCLRRKIPRGVAIVIAFFVSAVFHELCIAVPCHMFKLWAFFGIMFQIPLVVITNYFQRKFRSSMVGNMIFWFFFCILGQPMCVLLYYHDLMNRDGN

SEQ ID NO: 28 - protein, 446 aa, Populus trichocarpa
MMESEDLKSNGKECDKVTNENRSDIKFNYRPSMPAHRGVRESPLSSDAIFKQSHAGLFNLCIVVLVAINSRLIIENIIKYGWLINGGFWFSSKSLRDWPLFMCCLSLPAFPFAAYLVEKLAYQNYLPQLVVVFLHTIITTGSLLYPVLVILRCDSAFLSGVTLMLFSCIVWLKLVSYAHTNSDLRAIAKSIDREDVPSISPYVGNPYDTYFKSLVYFMVAPTLCYQSSYPRTESVRKGWVVQQFVKLIIFTGFMGFIIEQYINPIVKNSQHPFKGNLLYAIERVLKLSVPNLYVWLCMFYCFFHLWLNILAELLCFGDREFYKDWWNARTVEEYWRMWNMPVHKWMVRHIYFPCLRNKIPKGLAILIAFLVSAVFHELCIAVPCHVFKLWAFIGIMLQVPLVVITKFLQNKFRSSMVGNMIFWLFFSILGQPMCVLLYYHDLMNRK

SEQ ID NO: 29 - protein, 493 aa, Populus trichocarpa
MAESESPENRIAAMESTSSSTSDLNFSIRRRSTVMDSASTEMMGSEGLKSSGKACDKVKIEKQSDMKFNYRPSMPAHSGVRESPLSSDAIFKQSHAGLFNLCIVVLVAVNSRLIIENLIKYGWLINSGFWFSSKSLRDWPLFMCCLSLPAFPLAAYLVEKLAYRNCISELVVVFLHIIITTASLLYPVLVILRCDSALLSGGTLMLFACIVWLKLVSFAHTSSDMRAIAKSIDKENTPSISSKADNSYDANFKSLVYFMVAPTLCYQSSYPRSASVRKGWVVRQFVKLIIFTGFMGFIIEQYINPIVQNSQHPLKGNLLYAIERVLKLSVPNLYVWLCMFYCFFHLWLNILAELLRFGDREFYKDWWNARTVEEYWRMWNMPVHKWMVRHIYFPCLRNKIPKWAALLIAFFVSAVFHELCIAVPCHMFKLWAFIGIMFQVPLVVITKFLQNKFKSSMVGNMIFWLFFSILGQPMCVLLYYHDLMNRKGKTERR

SEQ ID NO: 30 - DNA, 2074 bp, Arabidopsis thaliana
  source 1..2074: mol_type = other DNA
tgaatcctttttcctttcttcttcttcttctcttcagagaaaactttgcttctctttctataaggaaccagacacgaatcccattcccaccgatttcttagcttcttccttcaatccgctctttccctctccattagattctgtttcctctttcaatttcttctgcatgcttctcgattctctctgacgcctcttttctcccgacgctgtttcgtcaaacgcttttcgaaatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgaaacaactgttcaaaaaatgactttcttcaaacatctatggcctcgttggatctccgttgatgttgtggtggttctgatgctaaaacgacaaatagtgttataaccattgaagaagaaaagaaaattagagttgttgtatctgcaaaaattttggtagagacacgcgaacccgtttggattttgttatggtgtaaagaaatttcaatcaaaaaactgttgtaataattgttaccaaaaagaaatgcttttctggaaacgaggggaaaaatagtagttttgtt

SEQ ID NO: 31 - DNA, 1512 bp, Brassica juncea
  source 1..1512: mol_type = other DNA
atggcgattttggattctggaggcgtcgctgtaccgccgacggagaacggcgtcgcggatctcgacaggctccaccgtcgtaaatcgagttcggattcttccaacggactcctctccgatacttccccgtcggacgatgttggagctgcggcggccgaaagggatcgggttgattccgctgccgaggaggaggctcagggaacagcgaatttagctggcggagatgccgaaactagggaatccgccggaggcgatgtaaggtttacgtatcgaccgtcggttccagctcatcggaggacgagggagagtcctctcagctccgacgctatcttcaaacaaagccatgcaggattgttcaacctctgtgtagttgttcttgttgctgttaacagtagactcatcatcgaaaacctcatgaagtatggttggttgatcagaactgatttttggtttagttctacatccttacgagactggccgcttttcatgtgttgtctttcactttcggtctttcctttggctgccttcacggtcgagaaaatggtacttcagaaattcatatctgagcctgttgccatcattcttcatgtcattataaccatgacagaggtcttgtatccagtctacgtcacactgaggtgtgattctgccttcttgtcaggtgtcacgttgatgctgctcacttgcattgtgtggctgaagttggtttcttacgctcatactagctacgacataagaaccctggccaattcagctgataaggtcgatcctgaaatctcctactatgttagcttgaagagcttggcgtatttcatggttgctcccacactgtgttatcagccaagctatccacgttctccatgtatccggaagggttgggtggctcgtcaacttgcaaaactggtcatattcactggactcatgggatttataatagagcaatatataaatcctattgttaggaactcaaagcatcctctgaaaggggaccttctatatgctattgaaagagtgttgaagctttcagttccaaatctatatgtgtggctctgcatgttctactgcttcttccacctttggttaaacatattggcagagctcctctgcttcggggaccgtgaattctacaaagattggtggaatgcaaaaagcgttggagattattggagaatgtggaatatgcctgttcacaaatggatggttcgacatgtatactttccgtgcctgcgcatcaagataccaaaagtacccgccattatcattgctttcttagtctctgcagtctttcatgagttatgcatcgcagttccttgccgtctcttcaatctatgggctttcatgggaattatgtttcaggtccctttggtctttatcacaaactttttacaagaaaggtttggctccatggtgggaaacatgatctttggttcagcttcttgcattttcggacaaccgatgtgtgggcttctttattaccatgacctgatgaaccgcaaaggatccatgtcctga

SEQ ID NO: 32 - DNA, 1512 bp, Brassica napus; identical to SEQ ID NO: 31
  source 1..1512: mol_type = other DNA

SEQ ID NO: 33 - DNA, 1510 bp, Brassica juncea
  source 1..1510: mol_type = other DNA
atggcgattttggattctggaactgtcacgatggcgacggagaacggtgtcgcggatctcgatatgcttcgtcgtcgtaaatcgagatcggattcttccaacggacttctctccgagacttccccatcggatgatgctggagctccggccgacgtggaggatcgggttgattcagctgctcagggaacagcgaatttagctggagatacggaaactagggaatccggtggaggaggaggaggaggaaacggcgaggtaaggtttacgtatcgaccgtcggttccagctcatcggaggacgagggagagtccactcagctccgacgctatcttcaaacaaagccatgcaggattgttcaacctctgtgtagttgttcttgttgctgttaacagtagactcatcatcgaaaatctcatgaagtacggttggttgatcagaactgatttctggtttagttctacatccctccgagattggccgcttttcatgtgttgtctttcactttcaatctttcctttggctgcctttaccgtcgagaaattggtactccagaaattcatatctgaacctgttgtcatcattcttcatattattatcaccatgactgaggtcttgtatccagtctacgtcaccctaaggtgtgattcggctttcttatcaggtgtcacattgatgctactcacttgcattgtgtggctgaagttggtttcttacgctcatactaattacgacataagaaccgtagctaattcagctgataaggtcgatcctgaagtctcctactatgttagcttgaagagcttggcgtatttcatggttgctcccacattgtgttatcagccaagctatccacgttctccgggtatccggaagggttgggtggctcgtcaatttgcgaaactggtcatattcactggactcatgggttttataatagagcaatatataaatcctattgtgaggaactcaaagcatcctttgaaaggggatcttctatacgctattgaaagagtgttgaagctttcagttccaaatctatatgtgtggctctgcatgttctactgcttcttccacctttggttaaatatattggcagagctcctttgcttcggggatcgtgaattctacaaagattggtggaatgcaaaaagcgtaggagattattggagaatgtggaatatgcctgttcataaatggatggttcgacatgtatactttccatgcctgcgcataaagataccgaaagtacccgccattatcattgctttcttagtctctgcagttttccatgagctgtgcattgcagttccttgccgtctctttaatttatgggctttcataggaatcatgtttcaggtgcctttggtctttatcacaaactatttacaagaaaggtttggctccatggtgggaaacatgatcttctggttcagcttctgcattttcggacaaccgatgtgtgtgcttctttattaccatgacctcatgaaccgcaaaggatccatgtcct

SEQ ID NO: 34 - DNA, 2090 bp, Tropaeolum majus
  source 1..2090: mol_type = other DNA
acgcggggagttttcaaaatcatattatgctttttcttcactactgcatgaactttctttctacttcttgcaactgatttgtaatccttacacatgtttctagttttctccatataaaaaaaatattctctgagcttctcgattctctagagagagaaggccaaaaaaaaatggcggtggcagagtcgtcacagaacacgacaaccatgagtggtcacggcgactcggatctcaacaatttccgtagaaggaaaccgagttcctccgtgattgaaccttcgtcgtccggttttacatccaccaatggcgtaccggcgactggccacgtggctgagaatcgtgaccaggatcgggtaggggctatggagaacgcaacaggatcggtcaacttaattggaaatggtggaggcgtggttatcgggaatgaagagaaacaggtaggggagactgatatacgattcacttaccggccttcgtttccggctcatcggagggtgagggagagtcctcttagctctgatgcaatcttcaaacagagccatgcgggtttattcaacttgtgtatagtagtgctcattgcagtaaacagtaggcttatcatcgaaaatcttatgaagtatggttggttgatcgatactggtttctggtttagctcaagatcactgggtgattggtccatctttatgtgctgtcttacactcccaattttcccacttgctgcttttattgttgaaaagctggtgcagcgaaatcatatatctgaacttgttgctgttctccttcatgtaatcgtttctaccgctgcagttttatatccagttattgtgatcttaacgtgtgattcggtgtatatgtctggtgtggtattgatgctctttggttgcattatgtggttgaagctggtgtcatatgcacatactagttctgatattagaacactggccaaatctggctataagggggatgcgcaccccaattcaaccattgtgagttgctcatatgatgttagcttgaagagtttggcatacttcatggttgctccgacattatgttaccagcctagctatcctcgttcgtcgtgtatccgcaagggttgggttgttcgtcaatttgtcaaactaatagttttcataggactcatggggttcattatagaacaatatattaatcctatcgttcgaaattccaaacacccattgaaaggagattttttatatgcaatagaaagagttttgaagctttcagttccaaatctatatgtttggctttgcatgttctactcttttttccacctctggttgaacatactggctgagcttcttcgctttggtgatcgtgaattctacaaagattggtggaatgcaaaaactgttgcggagtattggaaaatgtggaatatgcctgttcatagatggatggttcgtcatctatattttccctgtttgaggaatgggatacccaaggaaggtgccattattatcgcgttcttagtttctggtgctttccatgagctctgcattgcagttccttgccacgtattcaagttatgggcctttataggcattatgtttcaggttcccttggtattgattacgaattatctacaagaaaagttcagtaattctatggtgggcaatatgatcttctggttcatcttctgcatacttggccaacctatgtgtgtccttctatattaccatgacctgataaatctaaaggaaaagtgaaaaaatggaagttgcctatgctcagagtattcctatcccaatgcacacattatatggttctgtacaatctgtgcccccttcatcctttacacgtacccatgctggttcctgcacgatgatttgccttttgtttgtaagcaatatttggagagagtccaatttaggaagtgactagtgtggcttatatcttgtatactacctttagtcatgggggggtttttatattactagtaccaaaagtcaagttgtatatgatttacggtttagtttctttcatgttttttgtttttgtgtaaatatacgtttcatatatcactgttttttcaaagtaaaatcaataataccccatagatgttgaaactg

SEQ ID NO: 35 - DNA, 1494 bp, Echium pitardii
  source 1..1494: mol_type = other DNA
cacaaatgacaatatgggagtcgccggagatcataagctccgatgaagcagcagcggcgttgaggcggagaggcggcgccaaggaggttgcggaacagagattggatagtgaagaagagaagaagaaggaggaggaaaatggaaaactgaagtatacttatcgagcttccgctccggctcaccggagaatcaaggagagtcctcttagctccgacgccattttcaaacaaagtcatgcaggcctcttcaatctttgcattgtggtgcttgttgctgtaaacagtcggcttatcattgagaatttgatgaagtatggttggctaattagctcagggttttggttaagttcaacatcattaagcgactggccacttctaatatgttgtctcagtctgccgatattccctctggcttcttttgtggttgaaaagttgtctcaacaggaatttatatctgagcaagtggtgatcactcttcatgcactcataacaacgactgtgattatgtatccagtaattgtcatcctcagatgtgatcccgccgttctatcaggtgtgatattgatgcttttcacgtgtattgtgtggttgaagctggtatcttatgcacatacaaactatgatatgcgagcactagcaaaggactgtgataagttacaggcactatcaggctcttcaatggaagattgttcttttgaagtcaacttccaagctttggtatacttcatggttgctccgacattatgttatcagctaaggtatccgcgtaccccctgcattcgttggggttgggttacacgtcatctcatcaagttaatcatatttactggactgatgggattcattattgagcagtatattaatccaattgtgaaaaattcacaacatccattgaaggggaacctgttgtatgctatagagagggtcttgaagctttcagttccaaatatatatgtctggctctgcatgttttattgtctcttccatctttggttaaatatactagccgaacttctgtgttttggggatcgtgagttctacaaagattggtggaatgctcaaacgatagaggagtattggaggatgtggaacatgcctgtacataaatggatggttcgtcatatatatttcccttgcttgcggaatgggatgcctaaggagttggctattttgattgcgttcctaatatctgcaatcttccatgagctgtgcattgctgtgccgtgtcacatctttaagttctgggcttttatcggaataatgtttcaggtccctttggtccttctgacaaatgttttggtaaaaaagttccaaaattcaatggttggcaatatgatattctggtgcttcttctgcattcttggtcaacccatgagtctgctgctctattaccatgatgtcttgaatagaaaagttaatgcaaactgatactacagatatcttgaaaatgtcatcacaaagagtgtgaaggatcgataggtttcgctcaacagga

SEQ ID NO: 36 - DNA, 1828 bp, Vernonia galamensis
  source 1..1828: mol_type = other DNA
tctgagctcaaatcaaatttctgcgactcatacaggattcaactcaatactttcttgatcggttctgctgttcatttacttgtaatttctacttctgctttgctttcatttcaagcttttttccttaataatggcgttattagatacgcctcagattggagaaataacgaccaccgccaccacaactataagacggcggaccactgtcaagcctgatgctggaatcggagatggattgtttgattcttcgtcgtcttccaaaaccaactcatccttcgaggatggtgacagtttgaatggtgatttcaatgacaaatttaaggaacagatcggagctggtgatgaatccaaggacgactccaaggggaacggacagaagatagatcacggaggagttaaaaagggacgtgaaacgactgtggtgcattatgcttatcggccttcttctccggctcatcggagaattaaagaatctccgcttagctctgacgccatcttcaagcagagtcatgcaggcctctttaacctttgcatagtggtgcttgttgcagtaaatggtaggctcatcattgagaatctgatgaagtatggactattgatcaattccaacttttggttcagttcgagatcattgagagactggccacttctgatgtgctgcctcactccttctgactttccacttgctgcctacattgttgagaaattggcatggaaaaaacgtatatccgaccctgttgtaatcacactccatgttataataactacaactgcaattctttatccggtcttcatgattctgaggttcgattcagttgttctatcaggcgtctcgttgatgctgtgtgcttgcattaattggttgaagttggtatcttttgtgcatacaaattatgacatgcggtcgcttttgaactcaactgataagggagaagtggaacccatgtcttcaaatatggattatttttatgatgtcaacttcaaaagcttggtttatttcatggttgctccaactttgtgttaccagataagctatcctcgcactgcatttattcgaaagggttgggtgttacggcaactgatcaagctagtaatatttacagggttcatgggattcatcattgaacaatatatcaatccgattgtcaaaaattctcgtcatccattgaaaggagactttttatatgcgattgagcgggttttaaagctttcagttccgaatttatatgtgtggctctgtatgttctactgcttttttcacctttggttaaatatacttgctgagcttctttgttttggggatcgtgaattttataaagattggtggaatgcacaaactattgaagagtattggaggctatggaatatgcctgttcataaatggattgttaggcacctttattttccatgcttgcgtaatgggatacctaagggtgctgccatattggttgcatttttcatgtctgccgtgttccatgagctttgtattgctgttccctgccacattttcaagttttgggcttttatcgggatcatgtttcaggtcccgttggtcctactcacaaattacttgcagcacaagtttcaaaactcgatggtgggaaatatgatcttctggtgctttttcagcatttttggtcaacccatgtgtgtattactttactaccatgatgtcatgaatcaaaaggggaaaagcaaataaaaagatgtgattgtgttgctccatttgatctcatagcatgactggactaaacaaacccaagggacacattttagtccttaaaggaaaatttttgtaggaaaaaaaaaaaaaaaaaaaaaaaaaaaa

SEQ ID NO: 37 - DNA, 2099 bp, Nicotiana tabacum
  source 1..2099: mol_type = other DNA
ggcacgagcgaaatcttacccaatcctccgttgcttttcttttagatcctctttttctgtcattctctttttcccaataacaacaactcattgcatgtgaaggttgattttgatttttgtgtttattcaaactctctcttcacgattttcttactctttctagaagtatccattactttttagtctgtgattcggcgaaagtaagcaatggtgatcatggaattgccggagagcgtcgaaatgacgacgacgacgacgacttcgggtattgagaacctcaactccgatcttaatcactcggttcggaggagacgtggcagtaatggttttgaggcggctagtgcaattaacagttcggatgcgaatatgagcgaagatagaagagatgtgtgtggcagcggtgctggattggaaacggtgaatgagcggagtaaatcggttggtgagtccagtgatgtaattcgaaaggaggacgacaggaatgataatgttgcgaatggtgaggaaagcaaatcaacggaaacaacaacgacgccgtttaaatttgcttacagggcgtcggcaccagctcaccggcgaatcaaggagagtcctctcagctccgacgccattttcaaacagagtcacgcaggcctgttcaatctctgtgtggtggtgctgattgctgttaacagcaggctgattatcgagaacttgatgaagtatggccttttaattagggctggcttttggtttagctcgaagtcgttgagggattggccgcttctaatgtgctgtctcagtctccaaattttgccgctcgctgcttttcttgtggagaagttggcacagcagaggcatttgactgagcgtgcggtggttactcttcacataactataacaacagctgccattttgtatccagttctggtcattcttgggtgtgattctgcttttctgtttggtgtcatattgatgctggttgcttgcattgtgtggatgaagctggtttcttacgcacatacaaatcatgatatgagacagctcgcaaagtctacggacaaggatgaaacttcagatggggatttctcttatgatgttagcttcaagagtttggcttacttcatggttgcgccaacattatgttatcagcttagctatccccacactccatgcattcgcaaaggttgggtggcacgccaattcatcaagctggtaatatttacaggattgatgggatttatcatagaacagtacattaacccaattgtgcaaaactcacaacatcctttgaaaggaaaccttttatatgccatcgagagggtattgaagctttcggttccaaatttatatgtctggctctgcatgttttactgcttctttcatctttggctaaatatacttgcggaactactatgttttggtgatcgtgagttctacaaggattggtggaatgccaaaacaattgatgagtactggaggatgtggaatatgcctgttcataagtggatggttcgtcacatttatttcccttgcttaagaaacggaattccaaagggggtcgcaatactgattgctttccttgtatctgctgttttccacgagctgtgtattgctgttccatgtcgccttttcaagtggtgggcattcatgggaattatgttccaggttcctttggtcatactcacaaacttcttacaaaacaagttccaaagctcgatggtgggcaatatgatgttctggtgctttttctgcattcttggtcagccaatgtgtgtgcttctgtattaccacgatgtgatgaatagaaaaagcagtgcacgttaagcttcatccagggatgaattgttgtatgagcaagtattttaagttttggatcccaagctctattctactgtttctggcaaggcattcctgctatttccttcatcagttccaacaatattcagatgatacgaaatatctgtttggaatgcacaacacaagccacggccagagatgctgatgtctcacattttattgtgttcttcatgtcggagaaatgtaaaatactatcttgagataactctcatgttagtaaatacctttttgcctctaaaaaaaaaaaaaaaaaaaaaaaa

SEQ ID NO: 38 - DNA, 1964 bp, Perilla frutescens
  source 1..1964: mol_type = other DNA
tttagaaccaaactattctccgttaagttctgagttcgatttctttcttttctcaaattttccgtgcgatggcgatcttggactcgccggagatcctggacacgacgtcgtccagtgccgacaacggcgccgcacatcacaccactcttcgccggagacaaagtgcgcgctccgttccgcctcttctcgactccgattccaactctctggaggcagagagcgcaatcaatgattccgaaaatgttcgaaacgacgctaatttgatcgaaaatctccgcggcggagccgtggaatccgagaacgaaaaacaggagagttatggtaaggaggagggggcgaaagtgaaggagaatggagaaactagtaatggcaacggaactgatgttatggccgtcaaattcacattcaggccggcggcgcctgctcaccgcaaaaataaggagagtcctcttagctccgacgccatcttcaaacagagccatgcaggcctcttcaacctttgtatagtggtgcttgttgctgtaaatagcagactaataattgagaatttaatgaagtatgggtggctgatcaaatcaggattttggtttagttcaacatcgcttagggattggccactgctaatgtgttgtcttagtcttccagtttttgcactcgcttcatttcttgtcgagaagttggtgaaactaaattatatacctgagtgggtcgcagtctttcttcatgttacaatcacaacagtggaaatcttgtttccagttgttgtcattcttaggtgtgattctgctgttctatcaggtgtcacgctaatgctctttgcttgcactgtatggttgaagctcgtttcctacgcacatacaaactatgatttgagagtacttgcaaaatcacttgataagtgggaagctatgtccaggtactggaacctcgactacgcttatgatgtaagctttaagagtctggcatacttcatggttgctcctacattgtgttaccagccaagctaccctcggacagcttgcattcggaagggttgggtggtaaggcaactaattaagctggtaatattcacaggactcatgggatttattatagaacagtacataaacccgatcgttcaaaattctcaacatcctctgaaaggaaaccttttatatgccattgagagggtcttgaagctttctgttccaaatttatatgtgtggctctgcatgttttattgttttttccacctctggctaaatatacttgctgaacttctgtgctttggggaccgtgagttttataaggattggtggaatgcgaggacagtggaggagtactggagaatgtggaatatgcctgtccataaatggatggttcggcatatatactgtccatgcttacaaaatggaataccaaagatagtggcagttttgatcgcgtttcttgtgtctgcgatttttcatgagctgtgcgttgcagtcccttgccaaatattcaagttttgggcgttctcgggtatcatgcttcaggttcctctcgtaatcgtgactaattacttgcaagaaaagttcaaaaactcaatggtgggcaatatgatgttctggtgcttcttctgtatctttggtcaacctatgtgtgtgttgctgtactaccacgacttgatgaatcgaaaagctagtgcaaggtagggatgtgattcatcttctgagtagaaatctaaagctcaccagccccaacccacccgaaaaacaaaaaggagcaaggatcctgattgtgagctggtagataatttgctacaactatgtttcttaaatagctgggagtagtttgttatctgccttcacctaggacgacgttatgatctgttgtgatgggggtaagggggcatgcaaattttgtctatttttcaaggaatacagaaatggtgaaaatttgatgaagcatacccctcgtttactgacaaaaaaaaaaaaaaaaaa

SEQ ID NO: 39 - DNA, 1539 bp, Zea mays
  source 1..1539: mol_type = other DNA
atggcggactccgaagacgcgccgccagccgtgcaccgccgcccaccgcgccccgctcgcggtgctgctgcagcccagggcttcgcggccgcgttgcgccgccggctgagatccggcgctgcggtggcggcacgcgccagctttgccgcagactccggggacgagtctggccccggcgagccctcttcgtctcgccgccgcgacaacagcgggggcgcctcgtccgccgccggcggccgggccggggcaggggacttctccgcgttcaccttccgcgccgcggcgcctgtccaccggaaagccaaggagagccctctgagctccgacgccatcttcaagcagagtcatgcaggccttttcaacctatgtattgttgttctggttgcggtgaatagcaggctcattattgagaacctgatgaagtatggcttattaataagatctggcttttggtttaatgctacatcattgcgagactggccactgctaatgtgttgccttagtctacccatatttccccttggtgcatttgcagtcgaaaagttggcattcaacaatctcattagtgatcctgctactacctgttttcacatcctttttacaacatttgaaattgtatatccagtgctcgtgattcttaagtgtgattctgcagttttatcaggctttgtgttgatgtttattgcctgcattgtttggctgaagcttgtatcttttgcacatacaaaccatgatataagaaaactgatcacaagcggcaagaaggttgataatgaactgaccgcggctggcatagataatttacaagctccaactcttgggagtctaacatacttcatgatggctccgacactctgttatcagccaagttatcctcgaacaccttatgttagaaaaggttggctggtccgtcaagttattctatacttgatatttactggtctccaaggattcattattgagcaatacataaatcctattgttgtgaactctcaacatccattgatgggaggattactgaatgctgtagagactgttttgaagctctcattaccaaatgtctacctgtggctttgcatgttttattgccttttccatctgtggttaaacatacttgctgagattcttcgatttggtgaccgagaattctacaaagactggtggaatgcaaagacaattgatgagtactggagaaaatggaacatgcctgtgcataaatggattgttcgtcatatatatttcccttgcatgcgaaatggtatatcaaaggaagttgctgtttttatatcgttctttgtttctgctgtacttcatgagttatgtgttgctgttccctgccacatactcaagttctgggctttcttaggaatcatgcttcagattcccctcatcatattgacatcatacctcaaaaataaattcagtgacacaatggttggcaatatgatcttttggttttttttctgcatatacgggcagccaatgtgtgttctattgtattaccatgatgtgatgaaccggactgagaaggcaaaataa

SEQ ID NO: 40 - DNA, 1548 bp, Sorghum bicolor
  source 1..1548: mol_type = other DNA
atggcggacaccgacgacgcgccgccggccccggccgtgcaccgccgcccaccgcgccccgcccgcggtgctgccgctgcccagggcttcgcggccaagttgcgccgacggctcagctccggcgccgcggcggcggcgcgcgccagcttcgcggcagactccggggacgagtccggccccggggagccctcttcgtcccgccgccgcgacaacggcggggacgcctcgtccgccgccgacggcggccggggcggggcaggggacttctccgctttcacgttccgcgccgcggcgcctgtccaccggaaagccaaggagagccccctcagctccgacgctatcttcaagcagagtcatgcaggccttttcaacctatgtattgttgttctggttgcagtgaatagcaggctcattattgagaacctgatgaagtatggcttattaataaggtctggcttttggtttaatgctacatcattgcgagactggccactgctaatgtgttgccttagtctacctgtgtttccccttggtgcatttgcagttgaaaagttggcattcaacaatctcattactgatgctgccgctacctgttttcacatctttcttacaacacttgaaattgtatatccagtgcttgtgattcttaagtgtgattctgcagttttatcaggctttgtgttgatgtttattgcctgcattgtttggctgaagcttgtatcttttgcacatacaaaccatgatataagaaaactgatcacaagcggcaagaaggttgataatgaactgaccgtggctgacatagataatttacaagctccaactcttgggagtctaacatacttcatgatggctccgacactctgttatcagccaagttatcctcgaacaccttatgttagaaaaggttggctggttcgtcaagttattctatacttgatatttactggtctccaaggattcattattgagcaatacataaatcctattgttgtgaactctcaacatccattgaagggaggattactgaatgctgtagagactgttttgaagctctcattaccaaatgtctacctgtggctttgcatgttctattgccttttccatctatggttaaacatacttgctgagattcttcgatttggtgaccgtgaattctacaaagactggtggaatgcaaaaacaattgatgagtactggagaaaatggaacatgcctgtgcataaatggatgcttcgtcatatatattttccttgcatacgaaatggtatatcaaaggaagttgctgcttttatagcgttctttgtttctgctgtatttcatgagttatgtgttgctgttccctgccacatactcaagttctgggctttcttaggaatcatgcttcagattcccctcatcatactgacatcatacctcaaaaataaattcaatgacacaatggttggcaatatgatcttttggttctttttctgcatttacgggcagccaatgtgcgttctattgtattaccatgatgtgatgaaccggactgagaagacaaaataa

SEQ ID NO: 41 - DNA, 2103 bp, Oryza sativa
  source 1..2103: mol_type = other DNA
gtcgtcccgcgtctctcctcacctctcgtctccctcgcgacgtctccccttctccctccacccctccaatggtgggctccgatggcgacggcgacggcggcggaggggaagcacacgcgccggcggcgcccgcgcaccaccaccgccggcccccgcgcccgcggggaggcagcggggccatcgtcgagggcttcgcggcggcgctccgtcgcaggatccgctcgggggccgcggcggccgcgcgggccagcttcgggggcgactccggggacgaggccgcctccggggagccctcctcgtcctcgtcctcgtccccgtcccgccgccgtggcggcgactccaacggggcggaggcgtcctccgccgccgggggcggtggtggccgtggcggcggtggggacttctccgcgttcacgttccgcgcggcggcgccggtgcaccgcaaggccaaggagagccccctcagctccgacgccatcttcaagcagagtcatgcaggccttttcaacctatgcattgttgttctagttgcagtgaacagcaggcttattatcgagaacttaatgaagtatggcttattaataagagctgggttttggtttaatgataaatcattgcgggactggccacttctaatgtgttgtcttagtctgcctgctttccccctgggtgcatttgcagttgaaaagttggcatttaacaatgttattactgatgctgttgctacctgcctccatatcttcctttcaacaaccgaaattgtatatccagtgcttgtgattcttaagtgtgattctgcagttttgtctggctttttgttgatatttattgcctgtattgtttggctgaagcttgtatcttttgcacatacaaaccatgatataaggcaactgaccatgggcggcaagaaggttgataatgaactaagcacagttgacatggataatttacaacctccaactttagggaatctaatatacttcatgatggctcctacactctgttatcagccaagctatccccgaacttcatgtgttagaaaaggttggctgattcgtcaaattattctgtacttgatctttactggtcttcaaggcttcattattgagcaatacataaatccaattgttgtgaattctcagcatccattgaaaggaggactcctaaatgctgtagagactgttttgaaactctcattaccaaatgtttacctgtggctttgcatgttctatgcttttttccatctctggttaagtatacttgctgagattcttcgatttggtgaccgtgaattctacaaagattggtggaatgcaaaaacaattgatgagtattggagaaaatggaatatgcctgtacataaatgggttgttcgccatatttactttccttgcatgcgaaatggtatatcaaaggaagttgctgtcttgatatcattccttgtttctgccgtactccatgagatatgtgtcgctgttccctgccgcattctcaagttctgggcattcttaggaataatgctacagatcccccttatcgtattgacagcatacctcaaaagtaaattcagagatacaatggttggcaacatgatattttggttctttttctgcatctatgggcagccaatgtgccttctcctgtactatcatgatgtgatgaacaggattgagaaggcaagataaatgcgtgttgccatctttttcctctgtttcattttgtaccagcagaagcacaagcaataatccacatgctagccataaaacagcatgattcccaacggtgtggtacagccaaccttcctgttattctattttcttggctgtggtgtagatttagtttttaacttgtggctaaccgcaggaatgcctgtagataagcatctgtcattctgtctggcgacgttctccttattaatgtgtagatgtagaactgtttccgaaaactatatatcttgaatctgttatgcctcgacgaacataatccttttgttaagcttagttggtacagtctagaaaggataagagtcgtggatgtacgatttcgtctgccatatatcacgctcatattggcacaggtaactttgtcgctaccttctatctc

SEQ ID NO: 42 - DNA, 1473 bp, Oryza sativa
  source 1..1473: mol_type = other DNA
atggccccgcccccctcgctcgcccccgatcgcggcggcggcgaacccgacgacgccctccgcctgcgggcccgcgccgccgccgccgccggtgacgctcccgctccgcagcagcagcaggagcagaggcatcaggagcagcagcagcagctgctctggtaccgcgcgtcggcgcccgcccaccgccgcgtcagggagagccccctcagctccgacgccatcttccgccagagccatgcaggccttctgaacctatgcattgttgtgctggttgctgtgaacagcagacttattattgagaatttaatgaagtatggcctactaattagagctggattttggtttagtggaacatcgctggcagattggcctcttctcatgtgctgtctcactttaccaactttcccgcttgctgcacttatggttgagaagttggctcaaagaaaacttattagtaaacatgtggttattcttctccatatcgttattacaacatctgtccttgtctatccagttgttgtgattctaaagtgtgattcggcagtattatctggatttgtgttgatgtttcttgcaagcattatttggttgaagcttgtttcttttgctcatacaaattatgatataagaatgctgtccaaaagtattgaaaagggcgtgacacatgacatttctatagatccggagaacattaaatggccaacctttaaaaggctatcctacttcatgttggccccaacactttgttaccagccaagttatccccgaactacatatattagaaaaggttgggtggtccgacaactgataaaatgccttgtttttacaggcttgatgggttttataattgagcaatacataaatccaattgtgaagaattcgaagcatccattgaaagggaatttcttgaatgctatagagagagtattgaaattatcagtgccaacattatatgtctggctttgcatgttctactgttttttccatctctggttgaatattcttgctgagctcctctgttttggtgatcgtgaattctacaaggactggtggaatgccaaaacagttgaagagtattggagaatgtggaatatgcctgttcacaagtgggtcattcgacatatatattttccatgcataaggaatggtttttcaaagggtgttgctatcctaatctcgttcctggtttcagctgcatttcatgagatatgtattatttatttcattgtccttatcgatttgcagctatgtgttgctgttccatgccacatttttaaattctgggcatttattgggatcatgtttcagattcccctggtattcttgacgaaataccttcaagataaattcaataacacaatggtgggcaacatgatattttggttcttcttcagcatcctggggcaaccaatgtgtgttctcttatactaccatgatgtcatgaacaggcaacaagcccaaacaaatagatag

SEQ ID NO: 43 - DNA, 1590 bp, Sorghum bicolor
  source 1..1590: mol_type = other DNA
atggccccgcccccctccatggccgccgcctccgatcgcgccgtccccggcgccgacgcgaccgaggcgtcctccctccgcctccgccgcgccccctcagccgacgccggcgaccttgccgacgattcctcaggagaccggcgggagaacggcgagccgcaaccgccgcaggagcagcagcagcagcacgagatgctgtactaccgcgcgtcggcgcccgcccaccgccgcgtcaaggagagccccctcagctccgacgccatcttccggcagagccatgctggtcttctgaatctatgcatcgttgttctgattgcagtgaacagcagactcattattgagaatttaatgaagtatggcctattgataagagctggattttggtttagtgcaagatcgctgggtgactggccccttctaatgtgctgcctcactttaccagttttcccacttgttgccctcatggctgagaagctgattagaagaaagctcattggtgaacatgtggttattctactccatatcattattacaacatctgtcattgtctatccagttgttgtgactcttaagtgcgactcagcagtgctatctggattcttgctaatgtttcttgcgagcatcatgtggatgaagcttgtctcttatgcacatacaaattatgatataagggcattgtccaaaagtactgaaaagggtgctgcatatggaaattatgtcgatcctgagagtatgaaagatccaacctttaaaagtctagtgtacttcatgttggccccaacactttgttaccagccaacttatccccgaactacatgtattaggaagggttgggtgacccgacaacttataaagtgcctggtttttacaggcttgatgggcttcataattgagcaatatataaacccaattgtgaagaattccaaacatccactgaaagggaatttcttgaatgctatagaaagagtcttaaaactctcagtgccaacattatatgtatggctttgcatgttctattgcttttttcatttatggctgaacattctagctgaactcctctgtttcggtgaccgtgaattctacaaggactggtggaatgccaaaactgttgaagagtactggaggatgtggaacatgcctgttcataaatggatcatcagacacatatattttccatgtataaggaaaggcttttccaggggtgtagctattctagtctcgtttctggtttcagctgtatttcatgagatatgtattgcggtgccgtgccacattttcaaattctgggcattttctgggatcatgtttcagataccgttggtattcttgacaagatatctccaggctacgttcaagaatataatggtgggcaacatgatattttggttcttcttcagtatagtcgggcagccgatgtgtgtccttttatactaccatgatgtcatgaacaggcaggcccagcaagtagataattcggcagaaacatgtactttaagacaagttatcagaagcagactggagcgacgcagcaggaagcagcagcagcagcaggccagcagcccccctttgccattgttaccagctagctag

SEQ ID NO: 44 - DNA, 1485 bp, Zea mays
  source 1..1485: mol_type = other DNA
atggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatag

SEQ ID NO: 45 - DNA, 1912 bp, Physcomitrella patens
  source 1..1912: mol_type = other DNA
atgcctgtgaagagtagcaatctggcgggggagagggctgccaccagccatattaatgccaacacgaaattcgacctgcgggggtgtacgcctgcgcatcgcgttaggagggaaagcccgcttagttcagacgcaatttttcatcagagtcatgctgggttgttcaatctctgtattgttgttttgatagctgttaacagccggcttattattgagaacttaatgaagtatggactactcattagaactggtttctggtttagctccaagtctgcacgcgattggccactcttgatgtgcggtttgagtttaccgacttttccctttgcagcgttactagtggaaaaactatgctggaaaaatgaaaacgggaaatggttgattttcgtactacacctcataatcagcactgtagggatactgtatcctggatatgttatacacagggtgcaatccgcactgctgcctggtcttgtattgatactcattgcagtgactgggtggatgaagcttatatcttacgctcatgtcaacaaggacatgcgagaacttttgagagccaaagaaaagctacctgaggcaccacagtacgcagataaaatagaggttccggaccaccttacgatccaaaatattgcttatttcatgctcgcgcccacactttgctaccagttgagctaccctcgttcggacacaattcgaaaaagttgggtgttacggcaagccgggaaattggttgtgttcttgggtttagggggattcatcattgagcagtacataaatcccactgtgaagaattcacagcacccgctcaggggcaactatcttcaagcactggagagggttttaaagctttcgttgccagttctttatgtttggctgtgcttgttctactgtttatttcatctttggttgaatattgtggcggagctacttcgctttggggacagggaattttataaggactggtggaatgctcaaacagttgaagagtattggcgaatgtggaacatgcctgtgcacaagtggatggtgcggcatatttattttccctccattcgagctggcttatcaaagaaagcagcagtactactggtgtttgcaatttcagctttgtttcatgaggttatcattggtgttccgtgtcatatgcttcgatgctgggcttttcttggtatcatgatgcaggttccgttggtgtacttgacaaacgtgataaaagagcggtaccatagctctatggttggaaatatggtattttggttcttcttttgcattgtcgggcaacccatgtgcttgcttctctactaccacgacgttttcaacaactttcccagtacctgaactgagatcacccattcgtgcagttgttaatcttgtgaatagcactcctcatctgtaccttgttatggcttccacttctcgagaatcgactaaaccgcaactcatatgtttgtcaataacgattcattcgtaggcgggttggtgtgagattacaagagaggaaactttcgtcgtaagccagcagtgtagatacagtccaggtaggagtgtaacgggcacttgcttcaagggcagatccttgtcaacgcaagcttttgttggagcttgttggagcttgtttcgcacttagtattcttttctagctgtagttttaggtgacgttagtctattcttggtcccatcatccatcctgtaagatgctgggcgtgctcacgtgcagaagctgcctcgaatcctacgttacaatttcatgttggataccgcgtggatgtccgcttcagaatctgcgtcatgatgatgcacaccatttttttctattggaaactgaacagagggtagtgatacgtaagaacattttgggaaccgtgcctgaaaatcgtcggagcaaataatgatgtggttttgcagc

SEQ ID NO: 46 - DNA, 1278 bp, Selaginella moellendorffii
  source 1..1278: mol_type = other DNA
atgcggccctcgctcccggcgcaccggcggagcaaggagagcccgctcagctccgacgccatcttcacgcagagccacgccggcctcttcaacctctgcatcgtcgtgctggtggcggtgaacagccggctcatcatcgagaatctcatgaagtatggcctcctcatccaggccgaggtcctcttcagctccaagtcgctcaaggactggccgctcctcatgtgtggcctctcgctgctgatcttccccctcgccgcctatgtcatcgagaagatcaaggcccgccgccccgccaccgccgtggcgccgctccacttgatcaacctcgccgccgcgctgctctacccgatctacgtgatcgagatgttccagtcggatctcctctcggggctggttctcatgctcatcgccgtcaccggctggctcaagctcgtctcctacgcgcacaccaacgccgacatccgcgcggtgaagaaggacggtggcaagatcgagctccccgccgaggcgccggcgatcgactacccggacaacatctcgctcaagaacatcgcctacttcatggcagcgccgacgctgtgctaccagctgagctacccgcgctcgccgcggattagaaccgggtgggtgctccggcagctgggcaagtggatcgtcttcaacggattcatgggcttcatcatcgggcagtacatgaatccgatcatccggaactcgacgcacccgctcaaggggaactacctctacgcgatcgagcgcgtgctcaagctgtccatccccacgctctacgtctggctcggcttcttctactgcttcttccacctgtggctcaacatcgtggcggagatcctgtgcttcggcgaccgcgagttctacaaggactggtggaacgccaagtcggtggacgagtactggcggctgtggaacatgcccgtccaccgctggctcgtccgccacgtctacttcccgtgcctccggctgggcctccacaagcagttcgccattttggtggtgttcgtcatctccgggatctttcacgagatttgcatcgcggtgccgtgccacatgctgcggggctgggcgtttctggggatcatgttccaggtgccgctggttctggtgaccaacgtcctccagcgcaagttccagagctccatggtcggcaacatgatcttctggttcttcttttgcatcgtcgggcagccgatgtgcgtgctgctctactatcacgacgttgtcaacaggcagcagctccagctagctgggcggtccaaataa

SEQ ID NO: 47 - DNA, 1524 bp, Euonymus alatus
  source 1..1524: mol_type = other DNA
atggctgctaacttgaacgaagcctcggatcttaatttttcgcttcggaggagaactggtggcatctcaagtacgactgtgcctgattctagttccgagacaagttcgtcggaggcggactatttggacggaggcaaaggtgccgcggacgtcaaagatcgtggggatggtgcggtggagtttcagaattcgatgaagaacgtggagaggattgagaaacatgagagccgagtaggattggattcgagattcacgtataggccatcggtcccggctcatcgcacaataaaggagagcccgcttagctcggacgcaatattcaaacagagtcacgcaggtctcttcaatctctgtatagtagttctggttgctgtgaacagcaggctgatcattgaaaatctaatgaagtatggatggttaataaggagtgggttttggttcagctcaagatcattgagagactggcccctttttatgtgttgtctcacactaccagtattccctcttgctgcttttctgtttgagaagttggctcaaaaaaatttaatatctgaacctgttgttgttttgcttcatatagtaaacactacagctgccgttttataccctgttttggtgattctaaggtgtgattctgcctttatgtctggggttacgttgatgctctttgcttgtattgtgtggttaaagttggtatcttatgctcataccaactatgatatgagagccctcaccaagtctgttgaaaagggggacacgccgttgagctctcagaacatggattactcgtttgatgtcaatatcaagagtttggcatattttatggttgctcccacattatgttaccagattagctatcctcgtaccccatatgttcgcaagggttgggtggttcgtcaatttgtcaagttaataatatttactggacttatgggatttataattgaacaatatatcaatcctattgtccagaattcacaacaccctttgaaaggaaactttttgtatgccattgagagagttttgaagctttcagtcccaaacctttatgtttggctctgcatgttctactgcctttttcatctctggttaaacatacttgctgaacttctttgttttggtgatcgtgagttctacaaggattggtggaacgctaaaactgttgaggagtactggagaatgtggaacatgcctgttcataagtggatggttcgtcatatctacttcccatgcttgaggaacgggatacccaagggtgttgcttttgtcatttccttcttagtttctgccgtcttccatgagctatgcattgctgttccctgccacatcttcaagttatgggctttctttggaataatgcttcaggttcccttggtgttgatcacaagttatctgcaaaataagttcagaagctcaatggtgggaaatatgatgttttggttctctttctgcatttttggtcaacctatgtgcttacttctatattaccatgatttgatgaatcgcaatgggaagatggagtag

SEQ ID NO: 48 - DNA, 1557 bp, Vitis vinifera
  source 1..1557: mol_type = other DNA
atggcgatctgcaactcgcctgtcagtgtgaccacgtcgtcatcaagctctcacgccgattcagatctcgacttttccattcggaagaggttcggcgggaaggggaaggccgtggcggattcgtcgctggagacggagacggaggcggcggcggcggcggtgctcgaagcagagaagtcggtgggcgaggtggggagtggcggtgatcgaggggaatcggggagtcaggtggtgaggaatggggagaacggagtggctgaggttgccgcgaaattcgcgtaccggccgtgtgcgccggctcaccggaaagtgaaggaaagtcctctcagttctgacgccattttcagacagagtcatgcgggtctcttcaacctctgtatagtagtgcttgtagctgtaaacagccggcttatcattgagaatcttatgaagtatggttggttaatcagggctggtttttggtttagttcaaaatcattgagagattggccactctttatgtgctgtttaaccctcccaatctttccacttgctgcttttgtggttgaaaagttggctcaacaaaagtatatctctgagcaggttgttgtctctcttcacatcataattactacagctgcagttttgtttccagttttggtgattctaaggtgtgattcagctgttctctctggtgtcacactaatgctctttgcttgcattgtgtggttaaaattggtatcttttgcacatacaaattatgacatgagagcagttgccaagttaattgataagggggatgacttgtccacttcattgaatatggattacccttatgatgtcaacttcaagagtttggcatacttcatggttgcccccacgctatgttaccagccaagctatcctcgcagcacatgcattcggaagggttgggtctttcgccaatttgtcaagttggcaatatttacaggtgttatgggatttataatagaacagtatattaatccaattgttcagaattctcagcaccctttgaaggggaattttttttatgcattggagaggattttgaagctttctgttccaaatttatatgtgtggctctgcatgttctactgctttttccacctctggttaaatatacttgctgagcttcttcgttttggggaccgtgagttctataaagattggtggaatgcaaaaacagttgaggagtattggagaatgtggaatatgcctgttcataaatggatggttcgccatctctattttccatgtctacggaatgggatatctaagggagtttctgtggtgattgcctttgccatatctgccatattccatgagctatgcattgctgtaccttgtcacatgtttaagctttgggctttcattggaattatgttccaggttcccttggttttggtcacaaattacttgcaaaataagttcagaaattctatggtgggaaatatgatcttctggctgtttttcagcattcttggtcagccaatgtgtgtgcttctatattaccatgacttgatgaatcgaaaagagacaactgaatcaagcctctga

SEQ ID NO: 49 - DNA, 1880 bp, Glycine max
  source 1..1880: mol_type = other DNA
gaagagaagactgagttagtaaacacgctcgctcggtcttcttttccaatggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactgaaggtgcacgtggataagcttttctgtttttggagtgtataattgatgtcgatatgttgatcaatattggtttccacgagtactttcatctaccatggcagtggctgctctgaaggatttccacctgatataccaggtcgcgaggctaattcatcttgatctatgtacttaatcaactctcctctggcaattgtatcgatatatgcaattttgagagccatacactggcattgataactgccaaggaacagtgntagctgtttttctgttaaatgttaattagtagagagctagatgtaaataaatttatgctcaaaaaaaaaaaaaaaaaaaaaa

SEQ ID NO: 50 - DNA, 1960 bp, Glycine max
  source 1..1960: mol_type = other DNA
acgcggggggagaagagaagactgagttagaaaacacgctcggtcttcttctccaatggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagctcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattatccatgtttaaggcacggtctcccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatattcagtatccttggtcaacctatgtgtgtactgctatactaccatgacttgatgaataggaaaggcaaacttgactgaagctacggccattacattttaaaggtgcacatggatgagcttttcagttttcagattgtaaaattgatgtggatatgttggtcaatattgttttctacgaatgctttcatctaccatggcattggctgctctgaaggaattccacgggatatgccagttcacgaggctaattcattatcttgatctatgtacttaccaactctcctctggcaattgtatcaaaatatgcaattttgagagccatacactggcattgataactgccaaggaacactctaactgttttctgttagctgttaattagtagagggctagatgtaaatggtttatgctcaatatatttatttcctcctagtcttcaagttccaaaaaaaaaaaaaaaaaaaaaaa

SEQ ID NO: 51 - DNA, 1536 bp, Lotus japonicus
  source 1..1536: mol_type = other DNA
atggcgatttccgaagactctgaatctctattcgccgccgccgccgcttcctccgtcatccaaagcggctcttccgtacgccgcaggcccagcgctatctccgccgtcgcgacagtcgaagacgagagttcgagtgaagagccggtgccggtgagggattctggttccgacgtcgacgactccgtaagcagcgagcaacacgtctcccccgccaccgccaatcgagagaagaatcaggtgcatgacatctcagccaccaaattcgcctaccgtccttccgcccccgctcatcgcagagtcaaggagagccccctcagctccgacaacattttccgtcatcatgcgggtctcttcaacctttgtatagttgtgcttgttgcagtgaatagcagacttatcattgagaatttgatgaagtatggttggttgattaggactggcttttggtttagttcaaaatcattgagggattggccactcttcatgtgttgtctcagtcttgcaatatttccttttgccgcctttgtagtcgagaagttggtgcaacagaagtgtatttctgaaccagttgttgttcttcatatattcatttcaacagctgcagttgtctatccagttttagtaatcctcaggactgattctgcttttccatcaggcgtcacattaatgttatttgcttgcattgtatggctaaaactggtgtcttatgcacatacgaactatgatatgagagaacttaccaaatcaattgaaaagggagaagcacttcccaatactctgaatatggactattcttatgatgtgagcttcaagagcttggcatactttatgattgctcctacattatgttaccagccaagatatcctcgcagtccttctatccggaaaggttgggtgcttcgtcaacttgtcaagctgataatatttacaggagtaatgggatttataatagaacaatatattaatcctatagttcaaaattcacagcatcctttgaagggaaacctactatatgccattgaaagagttctgaagctgtctgttccaaacttatatgtgtggctctgcatgttctactgctttttccacctttggttaaatattctcgcagagcttcttagatttggtgatcgcgagttctacaaggattggtggaatgccaaaacttttgaagagtattggaggatgtggaatatgcctgttcacaaatggatgatccgacacctatattttccatgtttaagaaatggtatacccaagggtgttgctattttaattgccttcctggtttctgcattgttccatgagctgtgcattgctgttccttgccacattttcaagttgtgggcttttggtggaattatgtttcaggttcctttgatcttgataacaaattatctgcaaaataagttcagaaactcaatggttggaaacatgattttttggttcatattcagtattcttggtcaaccaatggccgtactgctatactaccacgatttgatgaatcggaaaagcaaacttgaccaaagctag

SEQ ID NO: 52 - DNA, 1620 bp, Medicago truncatula
  source 1..1620: mol_type = other DNA
atggcaatttccgatacaccggaaaccactgcaaccgccacagccaccgtaacaaccatcgaaaccgacacagatctcaaacgctcttcactccgacgacgaccgagcgccacatcaaccgccggtggtctcttcgacgcggaaagtgcagctgcagatgccgttcgagattcaggctccgatgattcgttgaacggtaagatcaacaacgaagaagaggttaaagatcgaaaaacggatcatgcagaaggaattgttgacgatgatgatgataatgcggttaagaagaatggtggtaatgacgtcatcaatgatcgtgaaaatgttgctgtcgatttcaaattcacttatcgtccttcagttcccgctcaccggagaagcaaagaaagtcctcttagctccggcaatatttttagacagagtcatgcaggactgttcaatctctgtattgtggtgcttgttgcagtgaatagtaggcttattattgaaaatctgatgaagtatggatggttgattcgatctggcttttggtttagttcaaaatcgcttagagattggcccctcttcatgtgttgtcttagtcttgcaatatttccacttgctgcctttgtagtcgaaaagttggcccaacaaaaacgtatttctgaaccagttattgttctccttcatattgtaattacaactgttatatcttatggatccatggccttgctgtgcaggtgcgattctgcttttttatctggttccacgttaatgctattgacttgcatagtgtggttaaaattggtgtcatatgcacatacaacctatgatatgagagcgcttgctgtttcaaatgaaaagggagaaacaatgcccgatactttcaatatggaggagtacccacacaatgtgagcttccagagtttagcatacttcatggttgctcctacattatgctaccagccaagctatcctcgcacaccttcggttcgaaagggttgggtctgtcgacaacttctcaagctggtcatatttacaggagttatgggatttataatagaacaatatatgaatcctattgtccagaattcacaacatccattgaagggaaaccttctatatgccattgagagagttctgaagctttctgttccaaatgtttatgtgtggctgtgcatgttctattgctttttccatctttggttaaatatacttgcggagcttctccggtttggtgatcgtgagttctacaaagattggtggaatgcccaaacggttgaagagtattggaggatgtggaatatgcctgtgcacaaatggatggttcgtcacgtgtattttccctgcataaggtttggtatacccaagggtgctgctgctttgactgctttcctggtttctgctgtgttccatgagttatgcattgctgttccttgccgcatgttcaagttgtgggcttttattggaattatgttccaggttcctttggtcttgatcaccaattacctgaaaaataaatacagaaactcaatggttggaaatatgattttttggttcatattttgtattcttggtcaacctatgtgtgtactactatactatcatgacttgatgaataggaaaggtgaaattgactga

SEQ ID NO: 53 - DNA, 1566 bp, Jatropha curcas
  source 1..1566: mol_type = other DNA
atgacgattttggagaccactactagcggaggtgatggtgttgctgagtcgtcttccgatcttaacgtatcgcttcgacggagacggaaaggcaccagctcggatggagctttgccggaattgacttcgaatattgttgaattggaatctgaaagcggtggccaggtgatgatggatccaggtatggtgacggaaccggagacagagaaaattaatggaaaagattgcggcggtgacaaggataagattgacaatcgcgagaatcgtgggaggtcggatattaaattcacgtaccggccatcggtgccggctcatcgagcgctcagggagagtccgcttagctctgatgctatatttaaacaaagtcatgcaggtctgttcaacctctgtatagtagtgcttgttgctgttaacagcaggcttatcattgaaaatctaatgaagtatggttggttaattaaaacggggttttggtttagttcaagatcgttgagagattggccccttcttatgtgctgtcttaccctccctatattctctcttgccgcctatctagttgagaagttggcatatcgaaaatatatatctgcacctattgttattttctttcatatgctcattaccacaacagcagttttgtacccagtttctgtgattctcagttgtgggtctgctgttctgtctggtgttgcattgatgctctttgcttgtatcgtgtggttgaaattagtatcttatgcacatacaaactatgacatgagagccattgccaactcagctgacaagggagatgcactatccgatacttcaggtgcagattcttcacgtgatgttagcttcaagagtttggtctacttcatggttgctcctacgctatgttaccagccaagttatcctcgaacagattcagttagaaagggttgggtggttcgtcaatttgtcaagttaataatatttacaggattcatgggatttatcatagaacaatatatcaatcctattgtccagaattcacaacaccccttaaagggggatctattatatgccattgaaagggttttgaagctctcagttccaaacttatatgtgtggctctgcatgttctactgcttttttcatctatggttaaatatacttgctgagctccttcggtttggtgacagagagttctataaagattggtggaatgcaaggacagttgaggagtactggagaatgtggaatatgcctgttcataagtggatggttcgccatatctactttccatgcttgcggcataaaataccaaggggggtagccttgttaattgctttcttcgtttcagctgtatttcatgagttgtgcattgctgttccttgccacatgttcaagctctgggcttttattggaattatgtttcagattccattggtcgggatcactaattacctccagaacaagttcagaagctccatggtgggaaatatgatcttttggttcattttctgcattcttggtcaacccatgtgtgtgctattgtattatcatgacctaatgaatcggaaaggcaatgctgaattaagatga

SEQ ID NO: 54 - DNA, 2084 bp, Vernicia fordii
  source 1..2084: mol_type = other DNA
tctctctctctttgctttacgtgtacatcgaccaccaccacagccatcttgcgactgttcaattatcctataagtaccaccgcattcatcaccgccaatccttaactctaatttgctatactaaacacttgctttatatgcgcttttctatttactcttcactgtaatttcttattggtattcaaagtgttttcaatgacaatccctgaaacgccggataattccacggatgctaccaccagtggcggtgctgagtcctcttccgatcttaacctttctcttcgacggaggaggactgcttcaaactccgatggagctgtcgcggaattggcttccaagattgatgagttggaatctgatgccggaggaggccaggtgattaaggatccgggagcagaaatggattcggggactttgaaaagtaatggaaaagattgcggaaccgttaaggataggattgaaaatcgtgagaatcgtggaggatcggatgttaaattcacgtatcggccgtcggtgccggctcaccgggcgctcaaggagagtccgcttagctctgataatatatttaaacaaagtcatgcaggtctcttcaatctctgtatagtagtgcttgtagcggttaacagtcggcttatcattgaaaacataatgaagtatggttggttaattaagactgggttttggtttagttcaagatcgttgagagactggccacttcttatgtgctgtcttaccctcccaatattttctcttgctgcctatctagttgagaagttggcctgtcggaagtatatatctgcacccactgttgtttttcttcatattcttttctcctcaacagcagttttataccctgtttctgtgattctcagttgtgaatctgctgttttgtccggtgtcgcattgatgctctttgcttgtatcgtgtggttgaaattggtatcttatgcacatacaaactttgatatgagagcaattgctaactcagttgataagggagatgcgctatccaatgcttcgagtgcagagtcctctcatgatgttagcttcaagagtttggtttatttcatggttgctcccacattgtgttaccagccaagttatcctcgaactgcatccattcgaaagggttgggtggttcgtcaatttgttaagttaataatatttacaggattcatgggatttatcatagaacaatatatcaatcctatcgttcagaattcacaacatcctttaaaaggggatctcttatatgccattgagagggttttgaagctctcagttccgaatttatatgtctggctttgcatgttctactgcttttttcacctatggttaaatatacttgctgagctccttcgctttggtgatagagagttctataaagattggtggaatgcaaggacagttgaggagtattggagaatgtggaatatgcctgttcataagtggatggttcgccatatctactttccatgcttgcggcataaaataccaaggggggtggccttattaattactttcttcgtttcagcagtatttcatgagttgtgcattgctgttccttgccacatattcaagctctgggcttttattggaataatgtttcagattcctttggtcgggatcacaaattaccttcaaaacaagttcagaagctcaatggtggggaacatgatcttctggttcattttctgcattcttggtcaaccaatgtgcttgctgttgtattaccatgacctaatgaatcgaaaagggactaccgaatcaagatgacactaactcatcgtgtggtagactctatatatatacatagacttaccagagatgggttgcttccaacatattgtgcacaagaggcaattgttgttctcatcagaagagtgggttaattaattaattaatgtacaagcaattttgaaagtataatcactggcagggactagtgcccgactgtagtactgagattatagaggtattatcaatcgttagtggaaaattgtaaatgtataaagttcaatctttgtattgtttcttttctaatatcatatttttttttattgctcatcaaaaaaaaaaaaaaa

SEQ ID NO: 55 - DNA, 1828 bp, Vernonia galamensis; identical to SEQ ID NO: 36
  source 1..1828: mol_type = other DNA

SEQ ID NO: 56 - DNA, 2020 bp, Ricinus communis
  source 1..2020: mol_type = other DNA
cttcttcttctgctgctgttcctctctcctccaccgccacgctcacctctctttgcatgacataatactattgttcttattatcattttcactctttaaatacaaacatcaattattccttttctatcaaacacatgtattctattctctcgtcgtctagattctcatcttcattgaatcctccttcttagcgtgtctttgtccacttcttttgggcaccgacgtttttaatctccatgacgattctcgaaacgccagaaactcttggcgtcatctcctcctccgccacttccgatctcaacctctctctccgacgtagacggacctcaaatgactccgatggtgcacttgctgatttggcttcgaagtttgatgatgatgacgacgtaagatcggaagattctgctgaaaatattatcgaagatcctgtagcagcggttactgaattggcgacagcaaagagtaacggaaaagactgtgttgccaatagtaataaggataaaattgatagccatggaggatcatcggattttaaacttgcatataggccttcggttccagctcaccggtcacttaaggagagtccgcttagctctgatttaatatttaaacaaagtcatgcaggtctgtttaacctttgtatagtagtgctcgtagctgttaacagcaggctcatcattgagaatttaatgaagtatggctggttaattaagacgggcttttggtttagttcaagatcattgagagattggccgctttttatgtgctgtctttctctcccagtattcccccttgctgcctatctagttgagaaggccgcatatcgaaaatatatatctccgcctattgttattttccttcatgtgatcatcacctcagcagctgttttgtacccagcttctgtaattctcagttgtgaatctgcttttttatctggtgtcacattgatggaacttgcttgtatggtatggttgaaattggtatcctatgcacatacaaactatgatatgagagcgatcgctgacaccattcataaggaagatgcatccaattcttctagtacagagtattgtcatgatgtgagctttaagactttggcgtacttcatggtcgcacccacattatgttaccagccaagttatcctcgcacagcatttattagaaagggctgggtgttccgtcaatttgtcaaactaataatttttacaggattcatgggatttatcatagaacaatacatcaatcctatcgtccagaattctcaacaccctttaaaaggggatctcttatatgccattgagagggttctgaagctctcagttccgaatttatatgtgtggctctgcttgttctactgcttttttcacctgtggttgaatatagttgctgagctccttcgcttcggtgaccgggagttctacaaagattggtggaatgcaaaaactgttgaggagtactggaggatgtggaatatgcctgttcacaagtggatggttcgccatatctacttcccatgcctacgtcgtaaaataccaaggggggtagcaatagttattgctttcttcgtttcagctgtatttcatgagttgtgcattgctgttccttgccacatgttcaaactttgggctttttttggaataatgtttcagattcctttagttgtgatcactaattattttcaaaggaagttcagaagctcaatggtgggaaatatgatcttctggttctttttctgcattctcggccaacctatgtgtgtactgttgtattaccatgacctaatgaatcgcgatgggaactgaaccatgggctcagtccagatatgggtacaccttccaagatgttattttcgtgagtgaagactgcaccacagtgttgttcttgttacacaatccccattgacagagtaggttaatcgtcagtttcaggagataagacacaattttgaaagtacagcagaggctgctattaatgtatcatgttgagtttctgttatgttatgttattcttttttaatctc

SEQ ID NO: 57 - DNA, 1444 bp, Populus trichocarpa
  source 1..1444: mol_type = other DNA
aaatcatggcggcgttagagtctccggagaatcatatacgacgtcggatcttaacttctccgttcgacggaggtctactactgtcacggattcgccttcgacggagatgatggaatcggaggatttgaaaagtaatggtaaagaatgcgataaggttacgaatgagaatcgatcggatattaaattcaattatcggccttcaatgcctgctcatcgtggtgttagagagagtcctcttagttctgatgctatttttaaacaaagtcatgcaggtctcttcaatctctgtatagttgtgcttgtggctataaacagcaggcttatcattgaaaatataatcaagtatggttggttaattaacggtggattttggtttagttcaaaatcattaagagactggcccctgtttatgtgctgtcttagccttccagcattccctttcgcggcctatcttgttgagaagttggcatatcaaaattatttacctcaacttgttgttgttttccttcatacaatcatcaccacaggatcacttttatatccagttttagtaattctcaggtgtgattctgcttttctatctggtgtcacgttgatgctcttttcttgcattgtgtggctaaaattggtatcttatgctcatacaaactctgatttgagagcaattgccaagtcaatagatagggaagatgtcccatccatttctccttatgtgggtaatccttatgatacttactttaagagtttggtctacttcatggtggctcccacattatgttaccagtcaagctatcctcgcactgaatctgttcgaaagggatgggtggttcaacaatttgtcaagttaataatatttactggattcatgggatttatcatagaacaatatatcaatcctattgttaagaattcacagcacccttttaaaggaaatctcttgtatgccattgagagggtcttgaagctctcagttcctaatttatatgtatggctttgcatgttctactgctttttccacctgtggttaaatatacttgccgagctcctttgctttggtgatcgggagttctacaaggattggtggaatgcaagaactgttgaagaatattggagaatgtggaatatgccagttcataagtggatggttcgccatatctattttccatgcctacggaataaaataccgaaggggttagctatacttattgccttcttagtttcagctgtatttcacgagctgtgcattgctgttccctgccacgtgttcaagctctgggcatttattggaattatgttacaggttcccttagtggtgatcacaaaatttctccaaaataagttcagaagctccatggtgggaaacatgatcttctggttgtttttcagcattcttggtcaaccaatgtgtgtgcttctgtattaccatgacttgatgaatcggaag

SEQ ID NO: 58 - DNA, 1816 bp, Populus trichocarpa
  source 1..1816: mol_type = other DNA
aacatatttaaatcatggcggagtcagagtcaccggagaatcgtatagcggcaatggaaagtacatcttcctcgacgtcagatctcaacttctctattcgacggaggtctacagtcatggactcggcttcgacggaaatgatgggatcggagggtttgaaaagtagtggtaaagcatgcgataaggttaagattgagaagcaatcagatatgaaattcaattatcggccatcaatgcccgctcatagtggtgttagagagagtcctcttagttctgatgctatttttaaacaaagtcatgcaggtctcttcaatctctgtatagtagtgcttgtggctgtaaacagcaggcttatcattgaaaatttaatcaagtacggctggttaatcaattcaggattttggtttagttcaaaatcattaagagactggcccctgtttatgtgctgtcttagtcttccagcattccctctcgcggcctatctcgttgagaagttggcatatcgaaattgtatatctgaacttgttgttgttttccttcatataatcatcaccacagcatcacttttgtatccagttttagtaattctcaggtgtgattctgctttactatctggtggcacattgatgctctttgcttgcattgtgtggttgaaattggtatcttttgcacatacaagctctgatatgagagcaattgccaagtcaattgataaggaaaataccccatccatttcttcgaaagcagataattcttatgatgctaactttaagagtttggtctacttcatggtggctcccacattatgttaccagtcaagctatcctcgttctgcatctgttcgaaagggttgggtggttcgacaatttgtcaagttaataatatttactggattcatgggatttatcatagaacaatatatcaatcctattgttcagaactcgcagcaccctttgaaaggaaatctcttgtatgccattgagagggtcttgaagctctcagttcctaatttatatgtttggctctgcatgttctactgctttttccacttgtggttaaatatacttgccgagctccttcgctttggtgatcgggagttctacaaggattggtggaatgcaagaactgttgaagagtactggagaatgtggaatatgccagttcataagtggatggttcgccatatctattttccatgtttacggaataaaataccaaagtgggcagccttacttattgccttctttgtctcagctgtatttcatgagttgtgtattgctgttccttgccacatgttcaagctctgggcatttattggaattatgtttcaggttcccttagtggtgatcacaaaattccttcaaaataagttcaaaagctcaatggtgggcaatatgatcttctggttatttttcagcattcttggtcaacctatgtgtgtgcttctatattaccatgacttgatgaatcggaaagggaaaactgaacgaagatgacaaatgcggtatggtagagatcgtcaagatgaacaaaatgcacggttatgatagtagagcaggcattaggtgtgccttttcttatgtattctgcaggagaaattgactcgattttgttgagtcgagagatggtctcttcaggacttttatttttatgtatctcaattgacgtgcaagcaattttggaagtacaagcactggcaattaaaatgccaatgcaacagtggatctgttgtgttggttaatcatttccagaaatttgtaaatgtttcttgttccgtcttttgcttcaaaggaaataaaaaaagaagaaaatttct

SEQ ID NO: 59 - DNA, 1656 bp, Tropaeolum majus
  source 1..1656: mol_type = other DNA
atggctgttgctgaatcttctcaaaacactactactatgtctggtcacggtgattctgatttgaacaacttcagaagaagaaagccatcctcctctgttattgaaccatcttcttcaggtttcacttctaccaatggtgttccagctactggtcatgttgctgaaaacagagatcaagatagagttggtgctatggaaaatgctactggttccgttaatttgattggtaatggtggtggtgttgtcatcggtaatgaagaaaaacaagttggtgaaaccgacatcagattcacttacagaccatcttttccagcccatagaagagttagagaatctccactctcgagtgatgctattttcaaacaatctcatgccggtttgttcaacttgtgcatcgttgttttgattgccgtcaactcaagattgatcatcgaaaacttgatgaagtacggttggttgattgatactggtttctggttctcttcaagatctttgggtgattggtctattttcatgtgttgtttgaccttgccaattttcccattggctgcttttatcgttgaaaagcttgttcaaagaaaccacatctccgaattggttgctgttttgttgcatgttatcgtttctactgctgctgtcttgtatccagttatcgttattttgacctgcgactccgtttacatgtctggtgttgttttgatgttgttcggttgcattatgtggttgaagttggtttcttacgctcacacctcttccgatattagaactttggctaaatctggttacaagggtgatgctcatccaaactctactatcgtttcttgctcttacgatgtctccttgaaatctttggcttactttatggttgctccaaccttgtgttatcaaccttcttatccaagatcctcctgcattagaaaaggttgggttgttagacaattcgtcaagttgatcgttttcatcggtttgatgggtttcatcatcgaacaatacatcaacccaatcgtcagaaactctaagcacccattgaaaggtgatttcttgtacgccattgaaagagtcttgaagctttctgttccaaacttgtacgtttggttgtgcatgttctactcattcttccatttgtggttgaacatcttggccgaattattgagattcggtgacagagaattttacaaggattggtggaatgctaagacagttgctgaatattggaagatgtggaacatgccagttcatagatggatggttagacacttgtacttcccatgtttgagaaacggtattccaaaagaaggtgccatcattatcgctttcttggtttctggtgctttccacgaattgtgtattgctgttccatgtcacgtttttaagttgtgggctttcattggtatcatgttccaagttccattggtcttgattaccaactacttgcaagaaaagttctccaactctatggtcggtaacatgattttctggttcattttctgcatcttgggtcaacctatgtgcgttttgttgtactaccatgacttgatcaacttgaaagaaaaggccaagggtgaattgagaggtcatccatttgaaggtaagccaattccaaatcctttgttgggtttggattctactagaactggtcatcatcatcaccatcac

SEQ ID NO: 60 - DNA, 1638 bp, Zea mays
  source 1..1638: mol_type = other DNA
atggctgattctgaagatgctcctccagctgttcataggagaccaccaagaccagctagaggtgctgctgctgctcaaggttttgctgctgctttgagaagaagattgagatctggagctgctgttgctgctagagcttcttttgctgctgattctggtgatgaatctggtccaggtgaaccatcttcttcaagaagaagagataattctggaggtgcttcttctgcagcaggtggtagagctggtgctggtgatttttctgcttttacttttagagctgctgctccagttcatagaaaagctaaagaatctccactctcgagtgatgctattttcaaacaatctcatgctggtttgtttaacttgtgtattgttgttttggttgctgttaactcaagattgattattgaaaacttgatgaagtacggtttgttgattagatctggtttttggtttaatgctacttctttgagagattggccattgttgatgtgttgtttgtctttgccaatttttccattgggtgcttttgctgttgaaaagcttgctttcaacaacttgatttctgatccagctactacttgttttcatattttgttcactactttcgaaattgtttacccagttttggttattttgaagtgtgattctgctgttttgtctggttttgttttgatgttcattgcttgtattgtttggttgaagttggtttcttttgctcatacaaaccatgatattagaaagttgattacttctggtaagaaagttgataatgaattgactgctgctggtattgataatttgcaagctccaactttgggttctttgacttactttatgatggctccaacattgtgttatcaaccatcttatccaagaactccatacgttagaaaaggttggttggttagacaagttattttgtacttgattttcactggtttgcaaggtttcattattgaacaatacattaacccaattgttgttaattctcaacatccattgatgggtggtttgttgaatgctgttgaaactgttttgaagctttctttgccaaatgtttatttgtggttgtgcatgttttactgtttgttccatttgtggttgaatattttggctgaaattttgagatttggtgatagagaattttacaaggattggtggaatgctaaaactattgatgaatattggagaaaatggaatatgccagttcataaatggattgttagacacatatacttcccatgtatgagaaatggtatttctaaagaagttgctgttttcatttcttttttcgtttctgctgttttacatgaattgtgtgttgctgttccatgtcatattttgaagttttgggcttttttgggtataatgttgcaaattccattgattattttgacatcttacttgaagaacaagttctctgatactatggttggtaatatgattttttggttcttcttctgcatatatggtcaaccaatgtgtgttttgttgtactatcatgatgttatgaacagaactgaaaaagctaaggccaagggcgagcttcgaggtcacccattcgaaggtaagcctatccctaaccctctcctcggtctcgattctacgcgtaccggtcatcatcaccatcaccat

SEQ ID NO: 61 - DNA, 1662 bp, Tropaeolum majus
  source 1..1662: mol_type = other DNA
atggctgttgctgaatcttctcaaaacactactactatgtctggtcacggtgattctgatttgaacaacttcagaagaagaaagccatcctcctctgttattgaaccatcttcttcaggtttcacttctaccaatggtgttccagctactggtcatgttgctgaaaacagagatcaagatagagttggtgctatggaaaatgctactggttccgttaatttgattggtaatggtggtggtgttgtcatcggtaatgaagaaaaacaagttggtgaaaccgacatcagattcacttacagaccatcttttccagcccatagaagagttagagaatctccactctcgagtgatgctattttcaaacaatctcatgctggtttgtttaacttgtgtattgttgttttggttgctgttaactcaagattgattattgaaaacttgatgaagtacggtttgttgattagatctggtttttggtttaatgctacttctttgagagattggccattgttgatgtgttgtttgtctttgccaatttttccattgggtgcttttgctgttgaaaagcttgctttcaacaacttgatttctgatccagctactacttgttttcatattttgttcactactttcgaaattgtttacccagttttggttattttgaagtgtgattctgctgttttgtctggttttgttttgatgttcattgcttgtattgtttggttgaagttggtttcttttgctcatacaaaccatgatattagaaagttgattacttctggtaagaaagttgataatgaattgactgctgctggtattgataatttgcaagctccaactttgggttctttgacttactttatgatggctccaacattgtgttatcaaccatcttatccaagaactccatacgttagaaaaggttggttggttagacaagttattttgtacttgattttcactggtttgcaaggtttcattattgaacaatacattaacccaattgttgttaattctcaacatccattgatgggtggtttgttgaatgctgttgaaactgttttgaagctttctttgccaaatgtttatttgtggttgtgcatgttttactgtttgttccatttgtggttgaatattttggctgaaattttgagatttggtgatagagaattttacaaggattggtggaatgctaaaactattgatgaatattggagaaaatggaatatgccagttcataaatggattgttagacacatatacttcccatgtatgagaaatggtatttctaaagaagttgctgttttcatttcttttttcgtttctgctgttttacatgaattgtgtgttgctgttccatgtcatattttgaagttttgggcttttttgggtataatgttgcaaattccattgattattttgacatcttacttgaagaacaagttctctgatactatggttggtaatatgattttttggttcttcttctgcatatatggtcaaccaatgtgtgttttgttgtactatcatgatgttatgaacagaactgaaaaagctaaggccaagggcgagcttcgaggtcacccattcgaaggtaagcctatccctaaccctctcctcggtctcgattctacgcgtaccggtcatcatcaccatcaccat

SEQ ID NO: 62 - DNA, 1395 bp, Zea mays
  source 1..1395: mol_type = other DNA
atgggtggtggttcttctgcttttacttttagagctgctgctccagttcatagaaaagctaaagaatctccactctcgagtgatgctattttcaaacaatctcatgctggtttgtttaacttgtgtattgttgttttggttgctgttaactcaagattgattattgaaaacttgatgaagtacggtttgttgattagatctggtttttggtttaatgctacttctttgagagattggccattgttgatgtgttgtttgtctttgccaatttttccattgggtgcttttgctgttgaaaagcttgctttcaacaacttgatttctgatccagctactacttgttttcatattttgttcactactttcgaaattgtttacccagttttggttattttgaagtgtgattctgctgttttgtctggttttgttttgatgttcattgcttgtattgtttggttgaagttggtttcttttgctcatacaaaccatgatattagaaagttgattacttctggtaagaaagttgataatgaattgactgctgctggtattgataatttgcaagctccaactttgggttctttgacttactttatgatggctccaacattgtgttatcaaccatcttatccaagaactccatacgttagaaaaggttggttggttagacaagttattttgtacttgattttcactggtttgcaaggtttcattattgaacaatacattaacccaattgttgttaattctcaacatccattgatgggtggtttgttgaatgctgttgaaactgttttgaagctttctttgccaaatgtttatttgtggttgtgcatgttttactgtttgttccatttgtggttgaatattttggctgaaattttgagatttggtgatagagaattttacaaggattggtggaatgctaaaactattgatgaatattggagaaaatggaatatgccagttcataaatggattgttagacacatatacttcccatgtatgagaaatggtatttctaaagaagttgctgttttcatttcttttttcgtttctgctgttttacatgaattgtgtgttgctgttccatgtcatattttgaagttttgggcttttttgggtataatgttgcaaattccattgattattttgacatcttacttgaagaacaagttctctgatactatggttggtaatatgattttttggttcttcttctgcatatatggtcaaccaatgtgtgttttgttgtactatcatgatgttatgaacagaactgaaaaagctaaggccaagggcgagcttcgaggtcacccattcgaaggtaagcctatccctaaccctctcctcggtctcgattctacgcgtaccggtcatcatcaccatcaccat

SEQ ID NO: 63 - DNA, 1656 bp, Tropaeolum majus
  source 1..1656: mol_type = other DNA
atggctgttgctgaatcttctcaaaacactactactatgtctggtcacggtgattctgatttgaacaacttcggtggtggtaagccatcctcctctgttattgaaccatcttcttcaggtttcacttctaccaatggtgttccagctactggtcatgttgctgaaaacagagatcaagatagagttggtgctatggaaaatgctactggttccgttaatttgattggtaatggtggtggtgttgtcatcggtaatgaagaaaaacaagttggtgaaaccgacatcagattcacttacagaccatcttttccagcccatagaagagttagagaatctccactctcgagtgatgctattttcaaacaatctcatgccggtttgttcaacttgtgcatcgttgttttgattgccgtcaactcaagattgatcatcgaaaacttgatgaagtacggttggttgattgatactggtttctggttctcttcaagatctttgggtgattggtctattttcatgtgttgtttgaccttgccaattttcccattggctgcttttatcgttgaaaagcttgttcaaagaaaccacatctccgaattggttgctgttttgttgcatgttatcgtttctactgctgctgtcttgtatccagttatcgttattttgacctgcgactccgtttacatgtctggtgttgttttgatgttgttcggttgcattatgtggttgaagttggtttcttacgctcacacctcttccgatattagaactttggctaaatctggttacaagggtgatgctcatccaaactctactatcgtttcttgctcttacgatgtctccttgaaatctttggcttactttatggttgctccaaccttgtgttatcaaccttcttatccaagatcctcctgcattagaaaaggttgggttgttagacaattcgtcaagttgatcgttttcatcggtttgatgggtttcatcatcgaacaatacatcaacccaatcgtcagaaactctaagcacccattgaaaggtgatttcttgtacgccattgaaagagtcttgaagctttctgttccaaacttgtacgtttggttgtgcatgttctactcattcttccatttgtggttgaacatcttggccgaattattgagattcggtgacagagaattttacaaggattggtggaatgctaagacagttgctgaatattggaagatgtggaacatgccagttcatagatggatggttagacacttgtacttcccatgtttgagaaacggtattccaaaagaaggtgccatcattatcgctttcttggtttctggtgctttccacgaattgtgtattgctgttccatgtcacgtttttaagttgtgggctttcattggtatcatgttccaagttccattggtcttgattaccaactacttgcaagaaaagttctccaactctatggtcggtaacatgattttctggttcattttctgcatcttgggtcaacctatgtgcgttttgttgtactaccatgacttgatcaacttgaaagaaaaggccaagggtgaattgagaggtcatccatttgaaggtaagccaattccaaatcctttgttgggtttggattctactagaactggtcatcatcatcaccatcac

SEQ ID NO: 64 - DNA, 1662 bp, Tropaeolum majus
  source 1..1662: mol_type = other DNA
atggctgttgctgaatcttctcaaaacactactactatgtctggtcacggtgattctgatttgaacaacttcggtggtggtaagccatcctcctctgttattgaaccatcttcttcaggtttcacttctaccaatggtgttccagctactggtcatgttgctgaaaacagagatcaagatagagttggtgctatggaaaatgctactggttccgttaatttgattggtaatggtggtggtgttgtcatcggtaatgaagaaaaacaagttggtgaaaccgacatcagattcacttacagaccatcttttccagcccatagaagagttagagaatctccactctcgagtgatgctattttcaaacaatctcatgctggtttgtttaacttgtgtattgttgttttggttgctgttaactcaagattgattattgaaaacttgatgaagtacggtttgttgattagatctggtttttggtttaatgctacttctttgagagattggccattgttgatgtgttgtttgtctttgccaatttttccattgggtgcttttgctgttgaaaagcttgctttcaacaacttgatttctgatccagctactacttgttttcatattttgttcactactttcgaaattgtttacccagttttggttattttgaagtgtgattctgctgttttgtctggttttgttttgatgttcattgcttgtattgtttggttgaagttggtttcttttgctcatacaaaccatgatattagaaagttgattacttctggtaagaaagttgataatgaattgactgctgctggtattgataatttgcaagctccaactttgggttctttgacttactttatgatggctccaacattgtgttatcaaccatcttatccaagaactccatacgttagaaaaggttggttggttagacaagttattttgtacttgattttcactggtttgcaaggtttcattattgaacaatacattaacccaattgttgttaattctcaacatccattgatgggtggtttgttgaatgctgttgaaactgttttgaagctttctttgccaaatgtttatttgtggttgtgcatgttttactgtttgttccatttgtggttgaatattttggctgaaattttgagatttggtgatagagaattttacaaggattggtggaatgctaaaactattgatgaatattggagaaaatggaatatgccagttcataaatggattgttagacacatatacttcccatgtatgagaaatggtatttctaaagaagttgctgttttcatttcttttttcgtttctgctgttttacatgaattgtgtgttgctgttccatgtcatattttgaagttttgggcttttttgggtataatgttgcaaattccattgattattttgacatcttacttgaagaacaagttctctgatactatggttggtaatatgattttttggttcttcttctgcatatatggtcaaccaatgtgtgttttgttgtactatcatgatgttatgaacagaactgaaaaagctaaggccaagggcgagcttcgaggtcacccattcgaaggtaagcctatccctaaccctctcctcggtctcgattctacgcgtaccggtcatcatcaccatcaccat

SEQ ID NO: 65 - DNA, 1662 bp, Arabidopsis thaliana
  source 1..1662: mol_type = other DNA
atggctattttggattctgctggtgttactactgttactgaaaacggtggaggtgaatttgttgatttggatagattgagaagaagaaagtcaagatctgattcttctaatggtttgttgttgtctggttctgataacaattctccatctgatgatgttggtgctccagctgatgttagagatagaattgattctgttgttaacgatgatgctcaaggcactgctaatttggctggtgataacaacggtggtggagacaacaatggtggtggaagaggaggtggtgaaggtagaggtaatgctgatgctacttttacttatagaccatctgttccagctcatagaagagctagagaatctccactctcgagtgatgctattttcaaacaatctcatgctggtttgtttaatttgtgtgttgttgttttgattgctgttaactcaagattgattattgaaaacttgatgaaatacggttggttgattagaactgatttttggttctcttcaagatctttgagagattggccattgtttatgtgttgtatttctttgtctatttttccattggctgcttttactgttgaaaagcttgttttgcaaaagtatatttctgaaccagttgttattttcttgcatattatcattactatgactgaagttttgtatccagtttacgttactttgagatgtgattctgcttttttgtctggtgttactttgatgttgttgacttgtattgtttggttgaagttggtttcttatgctcatacttcttacgatattagatctttggctaatgctgctgataaagctaatccagaagtttcttactacgtttctttgaaatctttggcttactttatggttgctccaactttgtgttatcaaccatcttatccaagatctgcttgtattagaaaaggttgggttgctagacaatttgctaagttggttattttcactggttttatgggtttcattattgaacaatacattaacccaattgttagaaattctaagcatccattgaaaggtgatttgttgtatgctattgaaagagttttgaagctttctgttccaaacttgtacgtttggttgtgcatgttttactgttttttccatttgtggttgaatattttggctgaattgttgtgttttggtgatagagaattttacaaggattggtggaatgctaaatctgttggtgattattggagaatgtggaatatgccagttcataaatggatggttagacacatatactttccatgtttgagatctaaaattccaaagactttggctatcattattgcttttttggtttctgctgtttttcatgaattgtgtattgctgttccatgtagattgtttaaattgtgggcttttttgggtataatgttccaagttccattggttttcattacaaactacttgcaagaaagatttggttctactgttggtaatatgattttctggttcattttctgtatttttggtcaaccaatgtgtgttttgttgtactatcatgatttgatgaatagaaagggttctatgtctgccaagggcgagcttcgaggtcacccattcgaaggtaagcctatccctaaccctctcctcggtctcgattctacgcgtaccggtcatcatcaccatcaccat

SEQ ID NO: 66 - DNA, 1662 bp, Arabidopsis thaliana
  source 1..1662: mol_type = other DNA
atggctattttggattctgctggtgttactactgttactgaaaacggtggaggtgaatttgttgatttggatagattgagaagaagaaagtcaagatctgattcttctaatggtttgttgttgtctggttctgataacaattctccatctgatgatgttggtgctccagctgatgttagagatagaattgattctgttgttaacgatgatgctcaaggcacttcaaatttggcttctgataacaacggtggtggagacaacaatggtggtggaagaggaggtggtgaatcaagatctaatgctgatgctacttttacttatagaccatctgttccagctcatagaagagctagagaatctccactctcgagtgatgctattttcaaacaatctcatgctggtttgtttaatttgtgtgttgttgttttgattgctgttaactcaagattgattattgaaaacttgatgaaatacggttggttgattagaactgatttttggttctcttcaagatctttgagagattggccattgtttatgtgttgtatttctttgtctatttttccattggctgcttttactgttgaaaagcttgttttgcaaaagtatatttctgaaccagttgttattttcttgcatattatcattactatgactgaagttttgtatccagtttacgttactttgagatgtgattctgcttttttgtctggtgttactttgatgttgttgacttgtattgtttggttgaagttggtttcttatgctcatacttcttacgatattagatctttggctaatgctgctgataaagctaatccagaagtttcttactacgtttctttgaaatctttggcttactttatggttgctccaactttgtgttatcaaccatcttatccaagatctgcttgtattagaaaaggttgggttgctagacaatttgctaagttggttattttcactggttttatgggtttcattattgaacaatacattaacccaattgttagaaattctaagcatccattgaaaggtgatttgttgtatgctattgaaagagttttgaagctttctgttccaaacttgtacgtttggttgtgcatgttttactgttttttccatttgtggttgaatattttggctgaattgttgtgttttggtgatagagaattttacaaggattggtggaatgctaaatctgttggtgattattggagaatgtggaatatgccagttcataaatggatggttagacacatatactttccatgtttgagatctaaaattccaaagactttggctatcattattgcttttttggtttctgctgtttttcatgaattgtgtattgctgttccatgtagattgtttaaattgtgggcttttttgggtataatgttccaagttccattggttttcattacaaactacttgcaagaaagatttggttctactgttggtaatatgattttctggttcattttctgtatttttggtcaaccaatgtgtgttttgttgtactatcatgatttgatgaatagaaagggttctatgtctgccaagggcgagcttcgaggtcacccattcgaaggtaagcctatccctaaccctctcctcggtctcgattctacgcgtaccggtcatcatcaccatcaccat

SEQ ID NO: 67 - DNA, 1662 bp, Arabidopsis thaliana
  source 1..1662: mol_type = other DNA
atggctattttggattctgctggtgttactactgttactgaaaacggtggaggtgaatttgttgatttggatagattgagaagaagaaagtcaagatctgattcttctaatggtttgttgttgtctggttctgataacaattctccatctgatgatgttggtgctccagctgatgttagagatagaattgattctgttgttaacgatgatgctcaaggcacttctaatttggcttcagataacaacggtggtggagacaacaatggtggtggaagaagaagaagagaatcaagatctaatgctgatgctacttttacttatagaccatctgttccagctcatagaagagctagagaatctccactctcgagtgatgctattttcaaacaatctcatgctggtttgtttaatttgtgtgttgttgttttgattgctgttaactcaagattgattattgaaaacttgatgaaatacggttggttgattagaactgatttttggttctcttcaagatctttgagagattggccattgtttatgtgttgtatttctttgtctatttttccattggctgcttttactgttgaaaagcttgttttgcaaaagtatatttctgaaccagttgttattttcttgcatattatcattactatgactgaagttttgtatccagtttacgttactttgagatgtgattctgcttttttgtctggtgttactttgatgttgttgacttgtattgtttggttgaagttggtttcttatgctcatacttcttacgatattagatctttggctaatgctgctgataaagctaatccagaagtttcttactacgtttctttgaaatctttggcttactttatggttgctccaactttgtgttatcaaccatcttatccaagatctgcttgtattagaaaaggttgggttgctagacaatttgctaagttggttattttcactggttttatgggtttcattattgaacaatacattaacccaattgttagaaattctaagcatccattgaaaggtgatttgttgtatgctattgaaagagttttgaagctttctgttccaaacttgtacgtttggttgtgcatgttttactgttttttccatttgtggttgaatattttggctgaattgttgtgttttggtgatagagaattttacaaggattggtggaatgctaaatctgttggtgattattggagaatgtggaatatgccagttcataaatggatggttagacacatatactttccatgtttgagatctaaaattccaaagactttggctatcattattgcttttttggtttctgctgtttttcatgaattgtgtattgctgttccatgtagattgtttaaattgtgggcttttttgggtataatgttccaagttccattggttttcattacaaactacttgcaagaaagatttggttctactgttggtaatatgattttctggttcattttctgtatttttggtcaaccaatgtgtgttttgttgtactatcatgatttgatgaatagaaagggttctatgtctgccaagggcgagcttcgaggtcacccattcgaaggtaagcctatccctaaccctctcctcggtctcgattctacgcgtaccggtcatcatcaccatcaccat

SEQ ID NO: 68 - DNA, 1371 bp, Arabidopsis thaliana
  source 1..1371: mol_type = other DNA
atgggtggtggttctgctacttttacttatagaccatctgttccagctcatagaagagctagagaatctccactctcgagtgatgctattttcaaacaatctcatgctggtttgtttaatttgtgtgttgttgttttgattgctgttaactcaagattgattattgaaaacttgatgaaatacggttggttgattagaactgatttttggttctcttcaagatctttgagagattggccattgtttatgtgttgtatttctttgtctatttttccattggctgcttttactgttgaaaagcttgttttgcaaaagtatatttctgaaccagttgttattttcttgcatattatcattactatgactgaagttttgtatccagtttacgttactttgagatgtgattctgcttttttgtctggtgttactttgatgttgttgacttgtattgtttggttgaagttggtttcttatgctcatacttcttacgatattagatctttggctaatgctgctgataaagctaatccagaagtttcttactacgtttctttgaaatctttggcttactttatggttgctccaactttgtgttatcaaccatcttatccaagatctgcttgtattagaaaaggttgggttgctagacaatttgctaagttggttattttcactggttttatgggtttcattattgaacaatacattaacccaattgttagaaattctaagcatccattgaaaggtgatttgttgtatgctattgaaagagttttgaagctttctgttccaaacttgtacgtttggttgtgcatgttttactgttttttccatttgtggttgaatattttggctgaattgttgtgttttggtgatagagaattttacaaggattggtggaatgctaaatctgttggtgattattggagaatgtggaatatgccagttcataaatggatggttagacacatatactttccatgtttgagatctaaaattccaaagactttggctatcattattgcttttttggtttctgctgtttttcatgaattgtgtattgctgttccatgtagattgtttaaattgtgggcttttttgggtataatgttccaagttccattggttttcattacaaactacttgcaagaaagatttggttctactgttggtaatatgattttctggttcattttctgtatttttggtcaaccaatgtgtgttttgttgtactatcatgatttgatgaatagaaagggttctatgtctgccaagggcgagcttcgaggtcacccattcgaaggtaagcctatccctaaccctctcctcggtctcgattctacgcgtaccggtcatcatcaccatcaccat

SEQ ID NO: 69 - protein, 552 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 70 - protein, 546 aa, Zea mays
MADSEDAPPAVHRRPPRPARGAAAAQGFAAALRRRLRSGAAVAARASFAADSGDESGPGEPSSSRRRDNSGGASSAAGGRAGAGDFSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 71 - protein, 553 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 72 - protein, 465 aa, Zea mays
MGGGSSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 73 - protein, 552 aa, Tropaeolum majus
MAVAESSQNTTTMSGHGDSDLNNFGGGKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHISELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 74 - protein, 554 aa, Tropaeolum majus
MAVAESSQNTTTMSGHGDSDLNNFGGGKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 75 - protein, 554 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMSAKGELRGHPFEGKPIPNPLLGLDSTRTGHHHHHH

SEQ ID NO: 76 - protein, 554 aa, Arabidopsis thaliana
MAILDSAGVTTVTENGGGEFVDLDRLRRRKSRSDSSNGLLLSGSDNNSPSDDVGAPADVRDRIDSVVNDDAQGTSNLASDNNGGGDNNGGGRGGGESRSNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMSAKGELRGHPFEGKPIPNPLLGLDSTRTGHHHHHH

SEQ ID NO: 77 - protein, 554 aa, Arabidopsis thaliana
MAILDSAGVTTVTENGGGEFVDLDRLRRRKSRSDSSNGLLLSGSDNNSPSDDVGAPADVRDRIDSVVNDDAQGTSNLASDNNGGGDNNGGGRRRRESRSNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMSAKGELRGHPFEGKPIPNPLLGLDSTRTGHHHHHH

SEQ ID NO: 78 - protein, 457 aa, Arabidopsis thaliana
MGGGSATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMSAKGELRGHPFEGKPIPNPLLGLDSTRTGHHHHHH

SEQ ID NO: 79 - DNA, 1758 bp, Tropaeolum majus
  source 1..1758: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggagctatggaaaacgctaccggatctgttaaccttatcggaaacggtggaggtgttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 80 - DNA, 1740 bp, Zea mays
  source 1..1740: mol_type = other DNA
atggctgattctgaggatgctcctcctgctgttcatagaaggcctccaagacctgctagaggtgctgctgctgctgtaaaagaagatgttttttatttccagcaatgttacattgttatacgtataatgatgagtttagtgatcaagttcctctttgattcttctttcttgttgcagcaaggattcgctgctgctcttagaagaaggcttagaagcggagctgctgttgctgctagagcttctttcgctgctgattctggtgatgagtctggacctggtgagccttcttcatctaggcgtagagataactctggtggagcttcttctgctgctggtggtagagctggtgctggtgatttctctgctttcaccttcagagctgctgctcctgttcacagaaaggctaaagaatctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 81 - DNA, 1686 bp, Zea mays
  source 1..1686: mol_type = other DNA
atggctcctccaccttctatgcctgctgcttctgatagagctggacctggaagagatgctggcgattctgtaaaagaagatgttttttatttccagcaatgttacattgttatacgtataatgatgagtttagtgatcaagttcctctttgattcttctttcttgttgcagtcatctctcagacttagaagggctccatctgctgacgctggtgaccttgctggtgatagctctggtggacttagagaaaacggtgagcctcaatctcctactaaccctccacctcaagagcaacaacagcacgagatgctttactacagagcttctgctcctgctcatagaagagtgaaagaatctccactctcgagtgatgctatcttcagacagtctcatgctggacttctcaacctctgcatcgttgttcttatcgctgtgaacagcagacttatcatcgagaacctcatgaagtacggacttctcatcagagctggattctggttctctgctagatctcttggagattggcctcttcttatgtgctgtcttacccttcctgttttccctcttgttgctcttatggccgagaagcttatcactagaaagctcatcggagagcatgttgttatccttctccacatcatcatcactacctctgctatcgtttaccctgttgttgttacccttaagtgcgattctgctgttctttctggattcgtgcttatgttcctcgcttctatcatgtggatgaagctcgtttcttacgctcacaccaactacgatatcagagtgctctctaagtctactgagaagggtgctgcttacggaaactatgtggaccctgagaacatgaaggatcctaccttcaagtctctcgtgtacttcatgcttgctcctaccctttgttaccaacctacttaccctcagactacctgtatcagaaagggatgggttacccaacaactcatcaagtgcgttgtgttcactggacttatgggattcatcatcgagcagtacatcaaccctatcgtgaagaactctaagcaccctcttaagggaaacttcctcaacgctatcgagagagttctcaagctttctgttcctactctttacgtttggctctgcatgttctactgtttcttccacctttggctcaacatcgttgctgagcttctctgtttcggagatcgtgagttctacaaggattggtggaacgctaagactgttgaggaatactggcgtatgtggaacatgcctgttcataagtggatcatcaggcacatctacttcccttgcatcaggaagggattctctaggggagttgctatccttatctctttcctcgtttctgctgttttccatgagatctgtatcgctgttccttgtcacatcttcaagttctgggctttctctggtatcatgttccagatccctcttgttttccttaccagataccttcacgctactttcaagcacgttatggtgggaaacatgattttctggttctttttcagcatcgttggacagcctatgtgtgttcttctctactaccacgatgttatgaacagacaagctcaggcttctagggctaagggtgagcttagaggtcatcctttcgagggtaagcctatccctaaccctcttctcggtctcgattctactagaactggtcatcatcatcaccatcac

SEQ ID NO: 82 - DNA, 1740 bp, Zea mays
  source 1..1740: mol_type = other DNA
atggctgattctgaggatgctcctcctgctgttcatggatcacctccatcacctgctagaggtgctgctgctgctgtaaaagaagatgttttttatttccagcaatgttacattgttatacgtataatgatgagtttagtgatcaagttcctctttgattcttctttcttgttgcagcaaggattcgctgctgctcttagaagaaggcttagaagcggagctgctgttgctgctagagcttctttcgctgctgattctggtgatgagtctggacctggtgagccttcttcatctggaggtagagataactctggtggagcttcttctgctgctggtggtagagctggtgctggtgatttctctgctttcaccttcagagctgctgctcctgttcacagaaaggctaaagaatctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 83 - DNA, 1740 bp, Zea mays
  source 1..1740: mol_type = other DNA
atggctgattctgaggatgctcctcctgctgttcatagaaggcctccaagacctgctagaggtgctgctgctgctgtaaaagaagatgttttttatttccagcaatgttacattgttatacgtataatgatgagtttagtgatcaagttcctctttgattcttctttcttgttgcagcaaggattcgctgctgctctttcatctaggctttcaagcggagctgctgttgctgctagagcttctttcgctgctgattctggtgatgagtctggacctggtgagccttcttcatctggaggtagagataactctggtggagcttcttctgctgctggtggtagagctggtgctggtgatttctctgctttcaccttcagagctgctgctcctgttcacagaaaggctaaagaatctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 84 - DNA, 1677 bp, Tropaeolum majus
  source 1..1677: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggagctatggaaaacatcagattcacctacagaccatctttcccagctcacagaagagttagggagtctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 85 - DNA, 1677 bp, Tropaeolum majus
  source 1..1677: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgttggagctatggaaaacgctaccggatctgttaaccttatcggaaacggtggaggtgttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 86 - DNA, 1763 bp, Tropaeolum majus
  source 1..1763: mol_type = other DNA
tggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggatcaatggaaaacgctaccggatctgttaaccttatcggaaacggtggatcagttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 87 - DNA, 1677 bp, Tropaeolum majus
  source 1..1677: mol_type = other DNA
atggctcctccaccttctatgcctgcttcatctgatagagctggaccttcaagagatgctggcgattctgtaaaagaagatgttttttatttccagcaatgttacattgttatacgtataatgatgagtttagtgatcaagttcctctttgattcttctttcttgttgcagtcatctctcagacttagaagggctccatctgctgactcatctgaccttgctggtgatagctctggtggacttagagaaaacggtgagcctcaatctcctactaaccctccacctcaagagcaacaacagcacgagatgctttactacagagcttctgctcctgctcatagaagagtgaaagaatctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 88 - DNA, 1755 bp, Tropaeolum majus
  source 1..1755: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcaagccttctagctctgttatcgagccttcttcttctggattcacctctactaacggtgttcctgctactggacatgttgctgagaacagagatcaggatagagttggagctatggaaaacgctaccggatctgttaaccttatcggaaacggtggaggtgttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 89 - DNA, 1665 bp, Tropaeolum majus
  source 1..1665: mol_type = other DNA
atggctcctccaccttctatgcctgctgcttctgatagagctggacctggaagagatgctggcgattctgtaaaagaagatgttttttatttccagcaatgttacattgttatacgtataatgatgagtttagtgatcaagttcctctttgattcttctttcttgttgcagtcatctctcgctccatctgctgacgctggtgaccttgctggtgatagctctggtggacttagagaaaacggtgagcctcaatctcctactaaccctccacctcaagagcaacaacagcacgagatgctttactacagagcttctgctcctgctcatagaagagtgaaagaatctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 90 - DNA, 1764 bp, Tropaeolum majus
  source 1..1764: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggatctatggaaaacgctaccggatctgttaaccttatcggaaacagacgtagagttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcac

SEQ ID NO: 91 - DNA, 1677 bp, Tropaeolum majus
  source 1..1677: mol_type = other DNA
atggctcctccaccttctatgcctgctgcttcaagaagacgtggacctggaagagatgctggcgattctgtaaaagaagatgttttttatttccagcaatgttacattgttatacgtataatgatgagtttagtgatcaagttcctctttgattcttctttcttgttgcagtcatctctcagacttagaagggctccatctgctgacgctggtgaccttgctggtgatagcagacgtagacttagagaaaacggtgagcctcaatctcctactaaccctccacctcaagagcaacaacagcacgagatgctttactacagagcttctgctcctgctcatagaagagtgaaagaatctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 92 - DNA, 1686 bp, Zea mays
  source 1..1686: mol_type = other DNA
atggctcctccaccttctatgcctagaaggagagatagagctggacctggaagagatgctggcgattctgtaaaagaagatgttttttatttccagcaatgttacattgttatacgtataatgatgagtttagtgatcaagttcctctttgattcttctttcttgttgcagtcatctctcagacttagaagggctccatctgctgacgctggtgaccttgctggtgatagctctggtggacttagagaaaacggtgagcctcaatctcctactaaccctccacctcaagagcaacaacagcacgagatgctttactacagagcttctgctcctgctcatagaagagtgaaagaatctccactctcgagtgatgctatcttcagacagtctcatgctggacttctcaacctctgcatcgttgttcttatcgctgtgaacagcagacttatcatcgagaacctcatgaagtacggacttctcatcagagctggattctggttctctgctagatctcttggagattggcctcttcttatgtgctgtcttacccttcctgttttccctcttgttgctcttatggccgagaagcttatcactagaaagctcatcggagagcatgttgttatccttctccacatcatcatcactacctctgctatcgtttaccctgttgttgttacccttaagtgcgattctgctgttctttctggattcgtgcttatgttcctcgcttctatcatgtggatgaagctcgtttcttacgctcacaccaactacgatatcagagtgctctctaagtctactgagaagggtgctgcttacggaaactatgtggaccctgagaacatgaaggatcctaccttcaagtctctcgtgtacttcatgcttgctcctaccctttgttaccaacctacttaccctcagactacctgtatcagaaagggatgggttacccaacaactcatcaagtgcgttgtgttcactggacttatgggattcatcatcgagcagtacatcaaccctatcgtgaagaactctaagcaccctcttaagggaaacttcctcaacgctatcgagagagttctcaagctttctgttcctactctttacgtttggctctgcatgttctactgtttcttccacctttggctcaacatcgttgctgagcttctctgtttcggagatcgtgagttctacaaggattggtggaacgctaagactgttgaggaatactggcgtatgtggaacatgcctgttcataagtggatcatcaggcacatctacttcccttgcatcaggaagggattctctaggggagttgctatccttatctctttcctcgtttctgctgttttccatgagatctgtatcgctgttccttgtcacatcttcaagttctgggctttctctggtatcatgttccagatccctcttgttttccttaccagataccttcacgctactttcaagcacgttatggtgggaaacatgattttctggttctttttcagcatcgttggacagcctatgtgtgttcttctctactaccacgatgttatgaacagacaagctcaggcttctagggctaagggtgagcttagaggtcatcctttcgagggtaagcctatccctaaccctcttctcggtctcgattctactagaactggtcatcatcatcaccatcac

SEQ ID NO: 93 - DNA, 1758 bp, Tropaeolum majus
  source 1..1758: mol_type = other DNA
atggctgttgctgaaagaaggagaaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggagctatggaaaacgctaccggatctgttaaccttatcggaaacggtggaggtgttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcac

SEQ ID NO: 94 - protein, 552 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 95 - protein, 546 aa, Zea mays; identical to SEQ ID NO: 70

SEQ ID NO: 96 - protein, 528 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASRAKGELRGHPFEGKPIPNPLLGLDSTRTGHHHHHH

SEQ ID NO: 97 - protein, 546 aa, Zea mays
MADSEDAPPAVHGSPPSPARGAAAAQGFAAALRRRLRSGAAVAARASFAADSGDESGPGEPSSSGGRDNSGGASSAAGGRAGAGDFSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 98 - protein, 546 aa, Zea mays
MADSEDAPPAVHRRPPRPARGAAAAQGFAAALSSRLSSGAAVAARASFAADSGDESGPGEPSSSGGRDNSGGASSAAGGRAGAGDFSAFTFRAAAPVHRKAKESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 99 - protein, 525 aa, Tropaeolum majus
MAVAESSQNTTTMSGHGDSDLNNFRRRKPSSSVIEPSSSGFTSTNGVPATGHVAENRDQDRVGAMENIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 100 - protein, 525 aa, Tropaeolum majus
MAVAESSQNTTTMSGHGDSDLNNFRRRKPSSSVIVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 101 - protein, 554 aa, Tropaeolum majus
MAVAESSQNTTTMSGHGDSDLNNFRRRKPSSSVIEPSSSGFTSTNGVPATGHVAENRDQDRVGSMENATGSVNLIGNGGSVVIGNEEKQVGETDIRFTYRPSFPAHRRVRESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 102 - protein, 525 aa, Tropaeolum majus
MAPPPSMPASSDRAGPSRDAGDSSSLRLRRAPSADSSDLAGDSSGGLRENGEPQSPTNPPPQEQQQHEMLYYRASAPAHRRVKESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 103 - protein, 551 aa, Tropaeolum majus
MAVAESSQNTTTMSGHGDSDLNNFKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 104 - protein, 521 aa, Tropaeolum majus
MAPPPSMPAASDRAGPGRDAGDSSSLAPSADAGDLAGDSSGGLRENGEPQSPTNPPPQEQQQHEMLYYRASAPAHRRVKESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 105 - protein, 554 aa, Tropaeolum majus
MAVAESSQNTTTMSGHGDSDLNNFRRRKPSSSVIEPSSSGFTSTNGVPATGHVAENRDQDRVGSMENATGSVNLIGNRRRVVIGNEEKQVGETDIRFTYRPSFPAHRRVRESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 106 - protein, 525 aa, Tropaeolum majus
MAPPPSMPAASRRRGPGRDAGDSSSLRLRRAPSADAGDLAGDSRRRLRENGEPQSPTNPPPQEQQQHEMLYYRASAPAHRRVKESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 107 - protein, 528 aa, Zea mays
MAPPPSMPRRRDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASRAKGELRGHPFEGKPIPNPLLGLDSTRTGHHHHHH

SEQ ID NO: 108 - protein, 552 aa, Tropaeolum majus
MAVAERRRNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 109 - DNA, 1767 bp, Tropaeolum majus
  source 1..1767: mol_type = other DNA
atggctgttgctgaatcttctcagaacaccactactatgtctggacacggtgattctgacctcaacgtaaaagaagatgttttttatttccagcaatgttacattgttatacgtataatgatgagtttagtgatcaagttcctctttgattcttctttcttgttgcagaacttcagacgtagaaagccttctagctctgttatcgagccttcttcttctggattcacctctactaacggtgttcctgctactggacatgttgctgagaacagagatcaggatagagttggagctatggaaaacgctaccggatctgttaaccttatcggaaacggtggaggtgttgttatcggtaacgaggaaaagcaagttggagagactgatatcagattcacctacagaccatctttcccagctcacagaagagttagggagtctccactctcgagtgatgctatcttcaagcagtctcacgctggacttttcaacctctgcatcgttgttcttgttgctgtgaacagcagactcatcatcgagaacctcatgaagtacggacttctcatcagatctggattctggttcaacgctacctctcttagagattggcctcttcttatgtgctgtctctctcttccaatcttccctcttggtgctttcgctgttgagaagcttgctttcaacaacctcatctctgatcctgctactacttgcttccacatccttttcactaccttcgagatcgtttaccctgttctcgttatccttaaatgcgattctgctgttctttctggattcgtgctcatgttcattgcttgcatcgtttggcttaagctcgtttctttcgctcacactaaccacgatatcagaaagctcatcacctctggaaagaaggttgacaacgagcttactgctgctggaatcgataaccttcaggctcctactcttggatctctcacctacttcatgatggctcctaccctttgttaccaaccttcttaccctagaaccccttacgttagaaagggatggcttgttagacaggttatcctctaccttatcttcactggacttcagggattcatcatcgagcagtacatcaaccctatcgttgttaactctcagcatcctcttatgggaggacttcttaacgctgttgagactgtgcttaagctttctctccctaacgtttacctttggctctgtatgttctactgccttttccacctttggcttaacatccttgctgagatccttagattcggagacagagagttctacaaggattggtggaacgctaagactatcgatgagtactggcgtaagtggaacatgcctgttcataagtggatcgtgaggcatatctacttcccttgcatgagaaacggaatctctaaagaggttgccgttttcatctctttcttcgtgtctgctgttctccatgagctttgtgttgctgttccttgccacatccttaagttctgggctttccttggaatcatgcttcagatccctcttatcatccttaccagctacctcaagaacaagttctctgataccatggtgggaaacatgattttctggttctttttctgcatctacggacaacctatgtgtgttcttctctactaccacgatgttatgaacagaaccgagaaggccaaggctaagggtgagcttagaggtcatcctttcgagggtaagcctatccctaaccctcttctcggtctcgattctactagaactggtcatcatcatcaccatcactga

SEQ ID NO: 110 - DNA, 1680 bp, Tropaeolum majus
  source 1..1680: mol_type = other DNA
atggctcctccaccttctatgcctgctgcttctgatagagctggacctggaagagatgctggcgattctgtaaaagaagatgttttttatttccagcaatgttacattgttatacgtataatgatgagtttagtgatcaagttcctctttgattcttctttcttgttgcagtcatctctcagacttagaagggctccatctgctgacgctggtgaccttgctggtgatagctctggtggacttagagaaaacggtgagcctcaatctcctactaaccctccacctcaagagcaacaacagcacgagatgctttactacagagcttctgctcctgctcatagaagagtgaaagaatctccactctcgagtgatgctatcttcaagcagtctcacgctggacttttcaacctctgcatcgttgttcttatcgctgttaatagcagacttatcatcgagaacctcatgaagtacggatggcttatcgatactggattctggttctcttctcgttctcttggtgactggtctatcttcatgtgctgtcttactctccctatcttccctcttgctgctttcatcgttgagaagcttgttcagaggaaccatatcgctgagcttgttgctgttcttctccacgttatcgtttctactgctgctgttctctaccctgttatcgttatccttacctgcgattctgtttacatgtctggtgttgtgcttatgcttttcggatgcatcatgtggcttaagctcgtttcttacgctcacacctcttcagatatcagaaccctcgctaagtctggatacaaaggtgatgctcaccctaactctactatcgtgtcttgctcttacgatgtgtctcttaagtctctcgcttacttcatggttgctcctaccctttgttaccaaccttcttaccctagatctagctgcatcagaaagggatgggttgtgagacaattcgttaagctcatcgtgttcatcggacttatgggattcatcatcgagcagtacatcaaccctatcgtgagaaactctaagcaccctctcaagggagatttcctttacgctatcgagagagtgcttaagctttctgtgcctaacctttacgtttggctctgcatgttctactcattcttccacctttggcttaacatccttgctgagttgcttagattcggagacagagagttctacaaggattggtggaacgctaagactgttgctgagtactggaagatgtggaacatgcctgttcatagatggatggttaggcacctttacttcccttgtctcagaaacggaatccctaaagagggtgctatcatcattgctttcttggtgtctggtgctttccatgagttgtgtatcgctgttccttgtcacgttttcaagctctgggctttcatcggaatcatgttccaagttcctctcgttcttatcactaactacctccaagagaagttctctaacagcatggtgggaaacatgattttctggttcattttctgcatccttggacagcctatgtgtgttcttctctactaccacgatctcatcaacctcaaagagaaggctaagggtgagcttagaggtcatcctttcgagggtaagcctatccctaaccctcttctcggtctcgattctactagaactggtcatcatcatcaccatcactga

SEQ ID NO: 111 - protein, 554 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLVAVNSRLIIENLMKYGLLIRSGFWFNATSLRDWPLLMCCLSLPIFPLGAFAVEKLAFNNLISDPATTCFHILFTTFEIVYPVLVILKCDSAVLSGFVLMFIACIVWLKLVSFAHTNHDIRKLITSGKKVDNELTAAGIDNLQAPTLGSLTYFMMAPTLCYQPSYPRTPYVRKGWLVRQVILYLIFTGLQGFIIEQYINPIVVNSQHPLMGGLLNAVETVLKLSLPNVYLWLCMFYCLFHLWLNILAEILRFGDREFYKDWWNAKTIDEYWRKWNMPVHKWIVRHIYFPCMRNGISKEVAVFISFFVSAVLHELCVAVPCHILKFWAFLGIMLQIPLIILTSYLKNKFSDTMVGNMIFWFFFCIYGQPMCVLLYYHDVMNRTEKAKAKGELRGHPFEGKPIPNPLLGLDSTRTGHHHHHH

SEQ ID NO: 112 - protein, 525 aa, Tropaeolum majus
MAPPPSMPAASDRAGPGRDAGDSSSLRLRRAPSADAGDLAGDSSGGLRENGEPQSPTNPPPQEQQQHEMLYYRASAPAHRRVKESPLSSDAIFKQSHAGLFNLCIVVLIAVNSRLIIENLMKYGWLIDTGFWFSSRSLGDWSIFMCCLTLPIFPLAAFIVEKLVQRNHIAELVAVLLHVIVSTAAVLYPVIVILTCDSVYMSGVVLMLFGCIMWLKLVSYAHTSSDIRTLAKSGYKGDAHPNSTIVSCSYDVSLKSLAYFMVAPTLCYQPSYPRSSCIRKGWVVRQFVKLIVFIGLMGFIIEQYINPIVRNSKHPLKGDFLYAIERVLKLSVPNLYVWLCMFYSFFHLWLNILAELLRFGDREFYKDWWNAKTVAEYWKMWNMPVHRWMVRHLYFPCLRNGIPKEGAIIIAFLVSGAFHELCIAVPCHVFKLWAFIGIMFQVPLVLITNYLQEKFSNSMVGNMIFWFIFCILGQPMCVLLYYHDLINLKEKAKGELRGHPFEGKPIPNPLLGLDSTRTGHHHHHH

SEQ ID NO: 113 - protein, 157 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLNK

SEQ ID NO: 114 - protein, 140 aa, Brassica juncea
MAILDSGGVAVPPTENGVADLDRLHRRKSRSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRPIIENLMK

SEQ ID NO: 115 - protein, 140 aa, Brassica napus
MAILDSGGVAVPPTENGVADLDRLHRRKSSSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRLIIENLMK

SEQ ID NO: 116 - protein, 140 aa, Brassica juncea
MAILDSGTVTMATENGVADLDMLRRRKSRSDSSNGLLSETSPSDDAGAPADVEDRVDSAAQGTANLAGDTETRESGGGGGGGNGEVRFTYRPSVPAHRRTRESPLSSDAIFKQSHAGLFNLCVVVLVAVNSRLIIENLMK

SEQ ID NO: 117 - protein, 149 aa, Tropaeolum majus
MAVAESSQNTTTMSGHGDSDLNNFRRRKPSSSVIEPSSSGFTSTNGVPATGHVAENRDQDRVGAMENATGSVNLIGNGGGVVIGNEEKQVGETDIRFTYRPSFPAHRRVRESPLSSDAIFKQSHAGLFNLCIVVLIAVNSRLIIENLNK

SEQ ID NO: 118 - protein, 103 aa, Echium pitardii
MTIWESPEIISSDEAAAALRRRGGAKEVAEQRLDSEEEKKKEEENGKLKYTYRASAPAHRRIKESPLSSDAIFKQSHAGLFNLCIVVLVAVNSRLIIENLPMK

SEQ ID NO: 119 - protein, 152 aa, Vernonia galamensis
MALLDTPQIGEITTTATTTIRRRTTVKPDAGIGDGLFDSSSSSKTNSSFEDGDSLNGDFNDKFKEQIGAGDESKDDSKGNGQKIDHGGVKKGRETTVVHYAYRPSSPAHRRIKESPLSSDAIFKQSHAGLFNLCIVVLVAVNGRLIIENLNK

SEQ ID NO: 120 - protein, 165 aa, Nicotiana tabacum
MVIMELPESVEMTTTTTTSGIENLNSDLNHSVRRRRCSNGFEAASAINSSDANMSEDRRDVCGSGAGLETVNERSKSVGESSDVIRKEDDRNDNVANGEESKSTETTTTPFKFAYRASAPAHRRIKESPLSSDAIFKQSHAGLFNLCVVVLIAVNSRLIIENLNK

SEQ ID NO: 121 - protein, 163 aa, Perilla frutescens
MAILDSPEILDTTSSSADNGAAHHTTLRRRRSARSVPPLLDSDSNSLEAESAINDSENVRNDANLIENLRGGAVESENEKQESYGKEEGAKVKENGETSNGNGTDVMAVKFTFRPAAPAHRKNKESPLSSDAIFKQSHAGLFNLCIVVLVAVNSRLIIENLNK

SEQ ID NO: 122 - protein, 141 aa, Zea mays
MADSEDAPPAVHRRPPRPARGAAAAQGFAAALRRRLRSGAAVAARASFAADSGDESGPGEPSSSRRRDNSGGASSAAGGRAGAGDFSAFTFRAAAPVHRKAKESPLSSDAIFKQSHAGLFNLCIVVLVAVNSRLIIENLMK

SEQ ID NO: 123 - protein, 142 aa, Sorghum bicolor
MADTDDAPPAPAVHRRPPRPARGAAAAQUAAELRRRLSSGAAAAARASFAADSGDESGPGEPSSSRRRDNGGDASSAADGGRGGAGDFSAFIFRAAAPVHFEAKESPLSSDAIFEQSHALENLCIVVLVAVNSPLIIENLNK

SEQ ID NO: 124 - protein, 167 aa, Oryza sativa
MVGSDGDGDGGGGEAHAPAAPAHHHRRPPRPRGGSGATVEGFAAALRRPIRSGAAAAARASFGGDSGDEAASGEPSSSSSSSPSRRRGGDSNGAEASSAAGGGGGRGGGGDFSAFTFRAAAPVHRKAKESPLSSDAIFKQSHAGLFNLCIVVLVAVNSRLIIENLNK

SEQ ID NO: 125 - protein, 104 aa, Oryza sativa
MAPPPSLAPDRGGGEPDDALRLRARAAAAAGDAPAPQQQQEQRHQEQQQQLLWYRASAPAHRRVRESPLSSDAIFRQSHAGLLNLCIVVLVAVNSRLIIENLMK

SEQ ID NO: 126 - protein, 118 aa, Sorghum bicolor
MAPPPSMAAASDRAVPGADATEASSLRLRRAPSADAGDLADDSSGDRRENGEPQPPQEQQQQHEMLYYRASAPAHRRVKESPLSSDAIFRQSHAGLLNLCIVVLIAVNSRLIIENLMK

SEQ ID NO: 127 - protein, 122 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMK

SEQ ID NO: 128 - protein, 76 aa, Physcomitrium patens
MPVKSSNLAGERAATSHINANTKFDLRGCTPAHRVRRESPLSSDAIFHQSHAGLFNLCIVVLIAVNSRLIIENLMK

SEQ ID NO: 129 - protein, 51 aa, Selaginella moellendorffii
MRPSLPAHRRSKESPLSSDAIFTQSHAGLFNLCIVVLVAVNSRLIIENLMK

SEQ ID NO: 130 - protein, 136 aa, Euonymus alatus
MAANLNEASDLNFSLRRRTGGISSTTVPDSSSETSSSEADFLDGGKGAADVKDRGDGAVEFQNSMKNVERIEKHESRVGLDSRFTYRPSVPAHRTIKESPLSSDAIFKQSHAGLFNLCIVVLVAVNSRLIIENLNK

SEQ ID NO: 131 - protein, 146 aa, Vitis vinifera
MAICNSFPSVTTSSSSSHADSDLDFSIRKRFGGKGKAVADSSLETETEAAAAAVLEAEKSVGEVGSGGDRGESGSQVVRNGENGVAEVAAKFAYRPCAPAHRKVKESPLSSDAIFRQSHACGLFNLCIVVLVAVNSRLIIENLNNK

SEQ ID NO: 132 - protein, 128 aa, Glycine max
MAISDEPETVATALNHSSLRRRPTAAGLFNSPETTTDSSGDDLAKDSGSDDSISSDAANSQPQQKKQDTDFSVLKFAYRPSVPAHREVEESPLSSDTIFRQSHAGLFNLCIVVLVAVNSRLIIENLMK

SEQ ID NO: 133 - protein, 133 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLFNLCIVVLVAVNSRLIIENLMK

SEQ ID NO: 134 - protein, 139 aa, Lotus japonicus
MAISEDSESLFAAAAASSVIQSGSSVRRRPSAISAVATVEDESSSEEPVPVRDSGSDVDDSVSSEQHVSPATANREKNQVHDISATKFAYRPSAPAHRRVKESPLSSDNIFRHHAGLFNLCIVVLVAVNSRLIIENLNK

SEQ ID NO: 135 - protein, 167 aa, Medicago truncatula
MAISDTPETTATATATVTTIETDTDLKRSSLRRRPSATSTAGGLFDAESAAADAVRDSGSDDSLNGKINNEEEVKDRKTDHAEGIVDDDDDNAVKKNGGNDVINDRENVAVDFKFTYRPSVPAHRRSKESPLSSGNIFRQSHAGLFNLCIVVLVAVNSRLIIENLNK

SEQ ID NO: 136 - protein, 148 aa, Jatropha curcas
MTILETTTSGGDGVAESSSDLNVSLRRRRKGTSSDGALPELTSNIVELESESGGQVMMDPGMVTEPETEKINGKDCGGDKDKIDNRENRGRSDIKFTYRPSVPAHRALRESPLSSDAIFKQSHAGLFNLCIVVLVAVNSRLIIENLNK

SEQ ID NO: 137 - protein, 153 aa, Vernicia fordii
MTIPETPDNSTDATTSGGAESSSDLNLSLRRRRTASNSDGAVAELASKIDELESDAGGGQVIKDPGAEMDSGTLKSNGKDCGTVKDRIENRENRGGSDVKFTYRPSVPAHRALKESPLSSDNIFKQSHAGLFNLCIVVLVAVNSRLIIENINK

SEQ ID NO: 138 - protein, 153 aa, Ricinus communis
MTILETPETLGVISSSATSDLNLSLRRRRTSNDSDGALADLASKFDDDDDVRSEDSAENIIEDPVAAVTELATAKSNGKDCVANSNKDKIDSHGGSSDFKLAYRPSVPAHRSLKESPLSSDLIFKQSHAGLFNLCIVVLVAVNSRLIIENLNK

SEQ ID NO: 139 - protein, 79 aa, Populus trichocarpa
MMESEDLKSNGKECDKVTNENRSDIKFNYRPSMPAHRGVRESPLSSDAIFKQSHAGLFNLCIVVLVAINSRLIIENIIK

SEQ ID NO: 140 - protein, 120 aa, Populus trichocarpa
MAESESPENRIAAMESTSSSTSDLNFSIRRRSTVMDSASTEMMGSEGLKSSGKACDKVKIEKQSDMKFNYRPSMPAHSGVRESPLSSDAIFKQSHAGLFNLCIVVLVAVNSRLIIENLIK

(19) United States
(12) Patent Application Publication
(10) Pub. No.: US 2025/0297271 A1
(43) Pub. Date: Sep. 25, 2025

(54) MODIFIED ACYLTRANSFERASE POLYNUCLEOTIDES, POLYPEPTIDES, AND METHODS OF USE

(71) Applicant: AGRESEARCH LIMITED, Lincoln (NZ)

(72) Inventors: Nicholas John ROBERTS, Feilding (NZ); Somrutai WINICHAYAKUL, Palmerston North (NZ)

(21) Appl. No.: 18/719,650

(22) PCT Filed: Dec. 14, 2022
(86) PCT No.: PCT/IB2022/062175

§ 371 Date: Jun. 13, 2024

(30) Foreign Application Priority Data
Dec. 16, 2021 (AU) 2021904092

(51) Int. Cl. C12N 15/82; C12N 9/10; C12P 7/64

(52) U.S. Cl. CPC C12N 15/8247; C12N 9/1029; C12N 15/8205; C12P 7/64; C12Y 203/0102

(57) ABSTRACT

The invention provides a method for producing a modified DGAT1 protein, comprising targeted manipulation of at least one motif selected from: a) a motif of the formula selected from RR, RXR, and RXXR, b) a motif of the formula AXXXA, c) a motif of the formula AXXXG, d) a motif of the formula GXXXG, and e) a motif of the formula GXXXA, in the N-terminal region of the protein upstream of the acyl-CoA binding site of a DGAT1 protein, where R is arginine, alanine, G is glycine and X is any amino acid. The modified DGAT1 protein can be expressed in a cell or organism, to increase the production of lipid in the cell or organism. The invention also provides the modified DGAT1 protein, polynucleotides encoding the modified DGAT1 proteins, cells and compositions comprising the polynucleotides or modified DGAT1 proteins, and methods using the modified DGAT1 proteins to produce oil.

### CROSS REFERENCE TO RELATED APPLICATIONS

This application is the 35 U.S.C. § 371 national stage application of PCT Application No. PCT/IB2022/062175, filed Dec. 14, 2022, where the PCT claims priority to, and the benefit of, Australian Patent Application entitled “Modified acyltransferase polynucleotides, polypeptides, and methods of use” having serial no. 2021904092, filed Dec. 16, 2021, both of which are herein incorporated by reference in their entireties.

### TECHNICAL FIELD

The invention relates to compositions and methods for the manipulation of cellular lipid production.

### REFERENCE TO SEQUENCE LISTING

The Sequence Listing submitted Jun. 6, 2025, as an Extensible Markup Language file named “2025-06-06_41253_2_Replacement_Sequence_Listing,” created on Jun. 6, 2025, and having a size of 249,126 bytes is hereby incorporated by reference.

### BACKGROUND

Plant oil is an economically important product not only due to its broad utilization in the food industry and as a component of feed ingredients but it also has a wide range of applications as biofuels or in the manufacture of various nutraceutical and industrial products. Within the plant itself, oil is essential to carry out a number of metabolic processes which are vital to growth and development particularly during seed germination and early plant growth stages. Considering its value, there is a growing research interest within the biotechnology field to improve plant oil production and make the supply more sustainable.

The major component of plant oil is triacylglyceride (TAG). It is the main form of storage lipid in oil seeds and the primary source of energy for seed germination and seedling development. TAG biosynthesis via the Kennedy pathway involves sequential acylation steps starting from the precursor sn-glycerol-3-phosphate (G3P). Firstly, G3P is esterified by an acyl-CoA to form lysophosphatidic acid (LPA) in a reaction catalyzed by glycerol-3-phosphate acyltransferase (GPAT, EC 2.3.1.15). This is followed by a second acylation step catalyzed by lysophosphatidic acid acyltransferase (LPAT; EC 2.3.1.51) forming phosphatidic acid (PA), a key intermediate in the biosynthesis of glycerolipids. The PA is then dephosphorylated by the enzyme phosphatidic acid phosphatase (PAP; EC3.1.3.4) to release the immediate precursor for TAG, the sn-1,2-diacylglycerol (DAG). Finally, DAG is acylated in the sn-3 position by the enzyme diacylglycerol acyltransferase (DGAT; EC 2.3.1.20) to form TAG.

Since this last catalytic action is the only unique step in TAG biosynthesis, DGAT is termed as the committed triacylglycerol-forming enzyme. As DAG is located at the branch point between TAG and membrane phospholipid biosyntheses, DGAT potentially plays a decisive role in regulating the formation of TAG in the glycerolipid synthesis pathway (Lung and Weselake, 2006, Lipids. December 2006; 41 (12): 1073-88). There are two different families of DGAT proteins. The first family of DGAT proteins (“DGAT1”) is related to the acyl-coenzyme A:cholesterol acyltransferase (“ACAT”) and has been desbried in the U.A. at. U.S. Pat. Nos. 6,100,077 and 6,344,548. A second family of DGAT proteins (“DGAT2”) is unrelated to the DGAT1 family and is described in PCT Patention Publication WO 2004/011671 published Feb. 5, 2004. Other references to DGAT genes and their use in plants include PCT Publication Nos. WO2004/011,671, WO1998/055,631, and WO2000/001,713, and US Patent Publication No. 20030115632.

DGAT1 is typically the major TAG synthesising enzyme in both the seed and senescing leaf (Kaup et al., 2002, Plant Physiol. 129 (4): 1616-26; for reviews see Lung and Weselake 2006, Lipids. December 2006; 41 (12): 1073-88; Cahoon et al., 2007, Current Opinion in Plant Biology. 10:236-244; and Li et al., 2010, Lipids. 45:145-157).

Raising the yield of oilseed crops (canola, sunflower, safflower, soybean, corn, cotton, linseed, flax etc) has been a major target for the agricultural industry for decades. Many approaches (including traditional and mutational breeding as well as genetic engineering) have been tried, typically with modest success (Xu et al., 2008, Plant Biotechnol J., 6:799-818 and references therein).

Although liquid biofuels offer considerable promise, the reality of utilising biological material is tempered by competing uses and the quantities available. Consequently, engineering plants and microorganisms to address this is the focus of multiple research groups; in particular the accumulation of triacylglcerol (TAG) in vegetative tissues and oleaginous yeasts and bacteria (Fortman et al., 2008, Trends Biotechnol 26, 375-381; Ohlrogge et al., 2009, Science 324, 1019-1020). TAG is a neutral lipid with twice the energy density of cellulose and can be used to generate biodiesel a high energy density desirable biofuel with one of the simplest and most efficient manufacturing processes. Engineering TAG accumulation in leaves has so far resulted in a 5-20 fold increase over WT utilising a variety of strategies which includes: the over-expression of seed development transcription factors (LEC1, LEC2 and WRI1); silencing of APS (a key gene involved in starch biosynthesis); mutation of CGI-58 (a regulator of neutral lipid accumulation); and upregulation of the TAG synthesising enzyme DGAT (diacylglycerol O acyltransferase, EC 2.3.1.20) in plants and also in yeast (Andrianov et al., 2009, Plant Biotech J 8, 1-11; Mu et al., 2008, Plant Physiol 148, 1042-1054; Sanjaya et al., 2011, Plant Biotech J 9, 874-883; Santos-Mendoza et al., 2008, Plant J 54, 608-620; James et al., 2010, Proc Natl Acad Sci USA 107, 17833-17838; Beopoulos et al., 2011, Appl Microbiol Biotechnol 90, 1193-1206; Bouvier-Navé et al., 2000, Eur J Biochem 267, 85-96; Durrett et al., 2008, Plant J 54, 593-607. However, it has been acknowledged that to achieve further increases in TAG, preventing its catabolismmay be crucial within non oleaginous tissues and over a range of developmental stages (Yang and Ohlrogge, 2009, Plant Physiol 150, 1981-1989).

Positively manipulating the yield and quality of triacylglycderides (TAG) in eukaryotes is difficult to achieve. The enzyme diacylglycerol-O-acyltransferase (DGAT) has the lowest specific activity of the Kennedy pathway enzymes and is regarded as a ‘bottleneck’ in TAG synthesis.

Attempts have been made previously to improve DGAT1 by biotechnological methods, with limited success. For example Nykiforuk et al., (2002, Biochimica et Biophysica Acta 1580:95-109) reported N-terminal truncation of the Brassica napus DGAT1 but reported approximately 50% lower activity. McFie et al., (2010, JBC., 285:37377-37387) reported that N-terminal truncation of the mouse DGAT1 resulted in increased specific activity of the enzyme, but also reported a large decline in the level of protein that accumulated.

Xu et al., (2008, Plant Biotechnology Journal, 6:799-818) recently identified a consensus sequence (X-Leu-X-Lys-X-X-Ser-X-X-X-Val) within Tropaeolum majus (garden nasturtium) DGAT1 (TmDGAT1) sequences as a targeting motif typical of members of the SNF1-related protein kinase-1 (SnRK1) with Ser being the residue for phosphorylation. The SnRK1 proteins are a class of Ser/Thr protein kinases that have been increasingly implicated in the global regulation of carbon metabolism in plants, e.g. the inactivation of sucrose phosphate synthase by phosphorylation (Halford & Hardie 1998, Plant Mol Biol. 37:735-48. Review). Xu et al., (2008, Plant Biotechnology Journal, 6:799-818) performed site-directed mutagenesis on six putative functional regions/motifs of the TmDGAT1 enzyme. Mutagenesis of a serine residue (S197) in a putative SnRK1 target site resulted in a 38%-80% increase in DGAT1 activity, and over-expression of the mutated TmDGAT1 in Arabidopsis resulted in a 20%-50% increase in oil content on a per seed basis.

N-terminal deletion of the DGAT (WO/2014/068437) or generation of chimeric DGATs by combining monocotyledonous and dicotyledonous DGAT peptide sequences (WO/2014/068439) has resulted in substantial increases in FA content in both yeast and plant tissues. However, these interventions require relatively large scale changes to DGAT1 sequences and/or target genomes and may be viewed by the regulatory authorities in some countries as genetic manipulations requiring arduous regulatory process to be completed before useful products of such technology can be widely commercialised.

It would be beneficial to provide forms of DGAT1 with similar or improved capacity to increase cellular lipid production, with smaller changes to wild-type sequences, which could be conveniently introduced using less interventionst technologies such as gene editing.

It is an object of the invention to provide modified DGAT1 proteins, and methods for their use to increase cellular lipid production, which overcome one or more of the deficiencies of the prior art, and/or at least to provide the public with a useful choice.

### SUMMARY OF THE INVENTION

The inventors have for the first time identified the presence of certain specific motifs in the N-terminal region of DGAT1 proteins. Furthermore, the applicants have surprisingly shown that it is possible to increase the capacity of DGAT1 proteins to produce cellular lipid, by targeted manipulation of these motifs to produce modified DGAT1 proteins. The motifs have a formula selected from: RR, RXR, and RXXR, AXXXA, AXXXG, GXXXG and GXXXA where R is arginine, A is alanine, G is glycine, and X is any amino acid. The modified DGAT1 proteins of the invention can be expressed in cells, organisms, and in particular plants, to increase lipid accumulation. The targeted manipulation of these motifs, can also be advantageously and conveniently achieved by introducing relatively small changes to endogenous DGAT1 genes using gene-editing technologies, to increase lipid accumulation in the cells, organisms and in particular plants.

### Method for Producing a Modified DGAT1 Protein

In the first aspect the invention provides a method for producing a modified DGAT1 protein, the method comprising targeted manipulation of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
   in the N-terminal region of the protein upstream of the acyl-CoA binding site of a DGAT1 protein, where R is arginine, A is alanine, G is glycine and X is any amino acid.

In one embodiment the N-terminal region extends from the N-terminus of the DGAT1 protein to a position at least 1, preferably at least 2, more preferably at least 3 amino acids upstream of the conserved motif ESPLSS (Glu-Ser-Pro-Leu-Ser-Ser) in the acyl-CoA binding site of the DGAT1 protein.

### Manipulation of the Motifs

In one embodiment the manipulation alters the number or position of at least one of the motifs in the N-terminal region of the protein upstream of the acyl-CoA binding site.

Those skilled in the art will understand that the modification may involve inserting, removing, or replacing one or more amino acids in the in the N-terminal region of the DGAT1 protein to alter the number or position of at least one of the motifs. In this way, existing motifs can be removed, new motifs can be created, or the distance between existing motifs can be altered. In one embodiment, the position of the motif is relative to the acyl-CoA binding site.

In a further embodiment, the position of the motif is relative to the conserved E (Glu) in the conserved motif ESPLSS (Glu-Ser-Pro-Leu-Ser-Ser) in the acyl-CoA binding site of the DGAT1 protein.

In a further embodiment the position of the motif is relative to another of the motifs as described herein.

In a further embodiment the position of the motif is relative to another motif of the same kind as described herein.

The at least one amino acid that is inserted, removed, or replaced may be an arginine or any other amino acid.

In one embodiment the method comprises removing at least one of the motifs in the N-terminal region of the protein upstream of the acyl-CoA binding site.

In a further embodiment the method comprises adding, or creating, at least one of the motifs in the N-terminal region of the protein upstream of the acyl-CoA binding site.

In a further embodiment the method comprises altering the relative position of at least two of the existing motifs in the N-terminal region of the protein upstream of the acyl-CoA binding site.

In a preferred embodiment the modification is a replacement of the at leat one amino acid, such that the length of the DGAT1 protein is unchanged.

Although not preferred, the modification may involve deleting or inserting multiple amino acids to alter the relative position of existing motifs in the N-terminal region of the DGAT1 protein. Thus one or more contiguous stretches of amino acids can be removed, or one or more stretches of amino acids can be inserted accordingly.

### RR, RXR, and RXXR Di-Arginine Motifs

In one embodiment the motif has a formula selected from RR, RXR, and RXXR, where R is arginine, and X is any amino acid. These motifs are also known as di-arginine motifs. Thus in one embodiment the manipulated motif is a di-arginine motif.

In one embodiment di-arginine motif further comprises two additional amino acids preceeding the first arginine (R), wherein the additional amino acids are selected from aromatic and bulky hydrophobic amino acid residues. In one embodiment the first arginine is preceded by two aromatic amino acid residues. In a further embodiment, the first arginine is preceded by two bulky hydrophobic amino acid residues. In a further embodiment, the first arginine is preceded by an aromatic amino acid residue and a bulky hydrophobic amino acid residue. In a further embodiment, the first arginine is preceded by a bulky hydrophobic amino acid residue and an aromatic amino acid residue.

In one embodiment the aromatic amino acid residues are selected from: phenylalanine (F), tyrosine (Y), tryptophan (W), and histidine (H).

In one embodiment the bulky hydrophobic amino acid residues are seletecd from: alanine (A), glycine (G), isoleucine (I), leucine (L), methionine (M), phenylalanine (F), proline (P), tryptophan (W), tyrosine (Y) and valine (V).

In a further embodiment the bulky hydrophobic amino acid residues are seletecd from: leucine (L), isoleucine (I), methionine (M), phenylalanine (F), tryptophan (W) and tyrosine (Y).

In one embodiment the di-arginine motif is removed by deleting an arginine in the motif.

In a further embodiment the di-arginine motif is removed by replacing an arginine in the motif.

In one embodiment when an arginine is replaced, preferred amino acids to replace the arginine (R) include: residues that are not positively charged and do not contain either a bulky hydrophobic or aromatic side chain.

In a further embodiment when an arginine is replaced, preferred amino acids to replace the arginine (R) are selected from: glycine (G) and serine(S).

In a further embodiment the di-arginine motif can be removed, added or created, by removal or addition of one or two aromatic and bulky hydrophobic preceding the first arginine (R).

In one embodiment at least one of the two amino acid preceeding the first arginine in a di-arginine motif is removed or replaced.

In a further embodiment the efficacy of the di-arginine motif can be reduced by removal or addition of one or two aromatic and bulky hydrophobic preceding the first arginine (R). In one embodiment at least one of the two amino acid preceeding the first arginine in a di-arginine motif is removed or replaced.

### Replacement of Alanine (a) in AXXXA AXXXG and GXXXA Motifs

As discussed above the motifs can be manipulated and effectively removed by replacement of one or more amino acids in a motif.

In one embodiment when an alanine (A) is replaced in an AXXXA, AXXXG, or GXXXA motif, preferred amino acids to replace the alanine (A) include: residues other than alanine (A) or Glycine (G).

In a further embodiment when an alanine (A) is replaced in an AXXXA, AXXXG, or GXXXA motif, a preferred amino acid to replace the alanine (A) is serine(S).

In a further embodiment when an alanine (A) is replaced in an AXXXA, AXXXG, or GXXXA motif, a preferred amino acid to replace the alanine (A) is arginine (R).

### Replacement of Glycine (G) in AXXXG, GXXXG and GXXXA Motifs

In one embodiment when a glycine (G) is replaced in an AXXXG, GXXXG or GXXXA motif, preferred amino acids to replace the glycine (G) include: residues other than alanine (A) or Glycine (G).

In a further embodiment when a glycine (G) is replaced in an AXXXG, GXXXG or GXXXA motif, a preferred amino acid to replace the glycine (G) is serine(S).

In a further embodiment when a glycine (G) is replaced in an AXXXG, GXXXG or GXXXA motif, a preferred amino acid to replace the glycine (G) is arginine (R).

Structure of the DGAT1 Protein after Modification and Relative Similarity to the Unmodified DGAT1 Protein

In one embodiment the modified DGAT1 protein is at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the un-modified DGAT1 protein.

In one embodiment the N-terminal region of the modified DGAT1 protein is at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the N-terminal region of the un-modified DGAT1 protein.

In a further embodiment less than 20, preferably less than 19, more preferably less than 18, more preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acids are changed in the modified DGAT1 protein relative to the un-modified DGAT1 protein.

In a further embodiment less than 20, preferably less than 19, more preferably less than 18, more preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acids are deleted or removed in the modified DGAT1 protein relative to the un-modified DGAT1 protein.

In a further embodiment less than 20, preferably less than 19, more preferably less than 18, more preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acids are inserted or added in the modified DGAT1 protein relative to the un-modified DGAT1 protein.

In one embodiment, when the distance between two existing motifs is increased, that distance is increased by less than 20, preferably less than 19, preferably less than 18, preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acid residues.

In an alternative embodiment, when the distance between two existing motifs is increased, that distance is increased by at least one, preferably at least 2, preferably at least 3, preferably at least 3, preferably at least 4, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20 amino acid residues.

In one embodiment, when the distance between two existing motifs is decreased, that distance is decreased by less than 20, preferably less than 19, preferably less than 18, preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acid residues.

In an alternative embodiment, when the distance between two existing motifs is decreased, that distance is decreased by at least one, preferably at least 2, preferably at least 3, preferably at least 3, preferably at least 4, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20 amino acid residues.

Preferably the modification is not truncation from the N-terminus of the DGAT1 protein.

Preferably the modification does not involve producing a chimeric sequence by combining an N-terminal portion of a first DGAT1 protein with a C-terminal portion of a second DGAT1 protein.

### Functional Properties of the Modified DGAT1 Protein

In one embodiment the modified DGAT1 protein has a greater capacity to increase cellular lipid production than does the un-modified DGAT1 protein.

In one embodiment when the modified DGAT1 protein is expressed in a cell, the cell produces more lipid than a suitable control cell in which modified protein is not expressed.

In one embodiment the cell in which the modified DGAT1 protein is expressed produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid than does the control cell.

In one embodiment the modified DGAT1 protein has at least one of:
i) increased DGAT1 activity,
ii) increased stability, and
iii) altered oligomerisation properties,
   relative to the unmodified DGAT1.
   Method with Assessing and or Selection Step

In one embodiment the method includes a step of assessing the capacity of the modified DGAT1 protein to increase cellular lipid production relative to that of the un-modified DGAT1 protein.

In a further embodiment the method includes a step of selecting a modified DGAT1 protein with greater capacity to increase cellular lipid production than that of the un-modified DGAT1 protein.

In one embodiment the method includes a step of testing the modified DGAT1 protein for at least one of:
i) increased DGAT1 activity,
ii) increased stability, and
iii) altered oligomerisation properties,
   relative to the unmodified DGAT1.

In a further embodiment the method includes a step of selecting a modified DGAT1 protein with at least one of:
i) increased DGAT1 activity,
ii) increased stability, and
iii) altered oligomerisation properties,
   relative to the unmodified DGAT1.

### How the Modified DGAT1 Proteins can be Produced

Those skilled in the art will understand that there are many well-known methodologies that can be used to produce the modified DGAT1 proteins in accordance with the invention.

In one embodiment the modified DGAT1 protein is synthesised directly from consitituent amino acids.

In a preferred embodiment the modified DGAT1 protein is expressed from a polynucleotide encoding the modified DGAT1 protein. Methods for producing polynucleotides for expressing proteins are methods well know to those skilled in the art, and include use of cloning and recombinant DNA technologies. These technologies may involve modification of an existing DGAT1 polynucleotide. Alternatively the polynucleotide can be synthesised in its entiretly by methods commonly used by those skilled in the art, and available commercially as a service from numerous well-know providers (e.g. GeneArt, Thermo Fisher Scientific).

The modified DGAT1 protein can be expressed from the polynucleotide in vitro by methods well-known to those skilled in the art.

Alternatively, and more preferably, the polynucleotide can be expressed in a cell or organism to produce the modified DGAT1 protein.

In another preferred method of the invention, the endogenous genome of a cell or organism is modified by gene editing techniques to produce a modified endogenous DGAT1 polynucleotide which when expressed produces the modified DGAT1 protein in the cell or organism.

Thus in one embodiment the modified DGAT1 protein is produced by expression of a polynucleotide encoding the modified DGAT1 protein.

In an alternative embodiment the polynucleotide is expressed in vitro to produce the modified DGAT1 protein.

In a preferred embodiment the polynucleotide is expressed in a cell or organism to produce the modified DGAT1 protein.

In a further preferred embodiment the polynucleotide is a modified endogenous DGAT1 polynucleotide in a cell or organism, and the modified endogenous DGAT1 polynucleotide is expressed in the cell or organism to produce the modified DGAT1 protein.

Those skilled in the art will understand that these methods can also be applied to test and select modified DGAT1 proteins with the desirable functional properties described above.

### Modified DGAT1 Protein

In a further aspect the invention provides a modified DGAT1 protein, with an altered number or position of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
   in the N-terminal region of the protein upstream of the acyl-CoA binding site of a DGAT1 protein, where R is arginine, A is Alanine, G is Glycine and X is any amino acid.

In one embodiment the N-terminal region extends from the N-terminus of the DGAT1 protein to a position at least 1, preferably at least 2, more preferably at least 3 amino acids upstream of the conserved motif ESPLSS (Glu-Ser-Pro-Leu-Ser-Ser) in the acyl-CoA binding site.

In one embodiment the modified DGAT1 protein has at least one less of the at least one of the motifs in the N-terminal region of the protein upstream of the acyl-CoA binding site, than does the un-modified DGAT1 protein.

In a further embodiment the modified DGAT1 protein has at least one more of the at least one of the motifs in the N-terminal region of the protein upstream of the acyl-CoA binding site, than does the unmodified DGAT1 protein.

In a further embodiment the modified DGAT1 has altered relative position of at least two existing motifs in the N-terminal region of the protein upstream of the acyl-CoA binding site.

Those skilled in the art will understand that the modification may involve inserting, removing, or replacing one or more amino acids in the in the N-terminal region of the DGAT1 protein to alter the number or position of di-arginine motifs. In this way at least one motif can be removed or added/created.

The at least on amino acid that is inserted, removed, or replaced may be an arginine or any other amino acid.

In a preferred embodiment the modification is a replacement of an amino acid, such that the length of the DGAT1 protein is unchanged.

Although not preferred, the modification may be a deletion or insertion of multiple amino acids to alter the relative position of existing motifs in the in the N-terminal region of the DGAT1 protein. Thus one or more contiguous stretches of amino acids may have been removed, or one or more stretches of amino acids may have been inserted accordingly.

### RR, RXR, and RXXR Di-Arginine Motifs

In one embodiment the motif has a formula selected from RR, RXR, and RXXR, where R is arginine, and X is any amino acid. These motifs are also known as di-arginine motifs. Thus in one embodiment the manipulated motif is a di-arginine motif.

In one embodiment di-arginine motif further comprises two additional amino acids preceeding the first arginine (R), wherein the additional amino acids are selected from aromatic and bulky hydrophobic amino acid residues. In one embodiment the first arginine is preceded by two aromatic amino acid residues. In a further embodiment the first arginine is preceded by two bulky hydrophobic amino acid residues. In a further embodiment, the first arginine is preceded by an aromatic amino acid residue and a bulky hydrophobic amino acid residue. In a further embodiment, the first arginine is preceded by a bulky hydrophobic amino acid residue and an aromatic amino acid residue.

In one embodiment the aromatic amino acid residues are selected from: phenylalanine (F), tyrosine (Y), tryptophan (W), and histidine (H).

In one embodiment the bulky hydrophobic amino acid residues are seletecd from: alanine (A), glycine (G), isoleucine (I), leucine (L), methionine (M), phenylalanine (F), proline (P), tryptophan (W), tyrosine (Y) and valine (V).

In a further embodiment the bulky hydrophobic amino acid residues are seletecd from: leucine (L), isoleucine (I), methionine (M), phenylalanine (F), tryptophan (W) and tyrosine (Y).

In one embodiment the di-arginine motif has been removed by deleting an arginine in the motif.

In a further embodiment the di-arginine motif has been removed by replacing an arginine in the motif.

In one embodiment when an arginine has been replaced, preferred amino acids replacing the arginine (R) include: residues that are not positively charged and do not contain either a bulky hydrophobic or aromatic side chain.

In a further embodiment when an arginine has been replaced, preferred amino acids replacing the arginine (R) are selected from: glycine (G) and serine(S).

In a further embodiment the di-arginine motif may have been removed, added or created, by removal or addition of one or two aromatic and bulky hydrophobic preceding the first arginine (R).

In one embodiment at least one of the two amino acids preceeding the first arginine in a di-arginine motif has been removed or replaced.

### Replacement of Alanine (a) in AXXXA and AXXXG Motifs

As discussed above motifs may have been manipulated and effectively removed by replacement of one or more amino acids in a motif.

In one embodiment when an alanine (A) is replaced in an AXXXA, AXXXG, or GXXXA motif, preferred amino acids to replace the alanine (A) include: residues other than alanine (A) or Glycine (G).

In a further embodiment when an alanine (A) is replaced in an AXXXA, AXXXG, or GXXXA motif, a preferred amino acid to replace the alanine (A) is serine(S).

In a further embodiment when an alanine (A) is replaced in an AXXXA, AXXXG, or GXXXA motif, a preferred amino acid to replace the alanine (A) is arginine (R).

### Replacement of Glycine (G) in AXXXG, GXXXG and GXXXA Motifs

In one embodiment when a glycine (G) is replaced in an AXXXG, GXXXG or GXXXA motif, preferred amino acids to replace the glycine (G) include: residues other than alanine (A) or Glycine (G).

In a further embodiment when a glycine (G) is replaced in an AXXXG, GXXXG or GXXXA motif, a preferred amino acid to replace the glycine (G) is serine(S).

In a further embodiment when a glycine (G) is replaced in an AXXXG, GXXXG or GXXXA motif, a preferred amino acid to replace the glycine (G) is arginine (R).

### Structure of the Modified DGAT1 Protein and Relative Similarity to the Unmodified DGAT1 Protein

In one embodiment the modified DGAT1 protein is at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the un-modified DGAT1 protein.

In a further embodiment less than 20, preferably less than 19, more preferably less than 18, more preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acids are changed in the modified DGAT1 protein relative to the un-modified DGAT1 protein.

In one embodiment, when the distance between two existing motifs is increased, that distance is increased by less than 20, preferably less than 19, preferably less than 18, preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acid residues.

In an alternative embodiment, when the distance between two existing motifs is increased, that distance is in creased by at least one, preferably at least 2, preferably at least 3, preferably at least 3, preferably at least 4, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20 amino acid residues.

In one embodiment, when the distance between two existing motifs is decreased, that distance is decreased by less than 20, preferably less than 19, preferably less than 18, preferably less than 17, preferably less than 16, preferably less than 15, preferably less than 14, preferably less than 13, preferably less than 12, preferably less than 11, preferably less than 10, preferably less than 9, preferably less than 8, preferably less than 7, preferably less than 6, preferably less than 5, preferably less than 4, preferably less than 3, preferably less than 2 amino acid residues.

In an alternative embodiment, when the distance between two existing motifs is decreased, that distance is decreased by at least one, preferably at least 2, preferably at least 3, preferably at least 3, preferably at least 4, preferably at least 4, preferably at least 5, preferably at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20 amino acid residues.

Preferably the modification is not truncation from the N-terminus of the DGAT1 protein.

Preferably the modification has not produced a chimeric sequence by combining an N-terminal portion of a first DGAT1 protein with a C-terminal portion of a second DGAT1 protein.

### Functional Properties of the Modified DGAT1 Protein

In one embodiment the modified DGAT1 protein has a greater capacity to increase cellular lipid production than does the un-modified DGAT1 protein.

In one embodiment when the modified DGAT1 protein is expressed in a cell, the cell produces more lipid than a suitable control cell.

In one embodiment the cell in which the modified DGAT1 protetin is expressed produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid than does the control cell.

In one embodiment the modified DGAT1 protein has at least one of:
i) increased DGAT1 activity,
ii) increased stability, and
iii) altered oligomerisation properties,
   relative to the unmodified DGAT1.

In a further embodiment the modified DGAT1 protein is produced by a method of the invention.

In a further embodiment the modified DGAT1 protein was tested or selected as described above.

### Polynucleotide Encoding the Modified DGAT1 Protein

In a further aspect the invention provides a polynucleotide encoding a modified DGAT1 protein of the invention.

In one embodiment the encoded modified DGAT1 protein has one or more of the properties discussed above.

In a further embodiment the polynucleotide is not found in nature.

### Constructs

In a further embodiment the invention provides a genetic construct comprising a polynucleotide of the invention.

In one embodiment the construct comprises a promoter operably linked to the polynucleotide.

Those skilled in the art will understand that polynucleotides and constructs for expressing polypeptides in cells, plants and other organisms can include various other modifications including restriction sites, recombination/excision sites, codon optimisation, tags to facilitate protein purification, etc. Those skilled in the art will understand how to utilise such modifications, some of which may influence transgene expression, stability and translation. However, an art skilled worker would also understand that these modifications are not essential, and do not limit the scope of the invention.

The polynucleotide may also be an endogenous DGAT1 polynucleotide, that has been modified for example via a gene-editing technique, to encode the modified DGAT1 protein.

### Cells

In a further embodiment the invention provides a cell comprising a polynucleotide, construct or modified DGAT1 protein of the invention.

In one embodiment the polynucleotide is an transformed into the cell.

In a further embodiment the polynucleotide is an endogenous DGAT1 polynucleotide that has been modified in the cell to encode the modified DGAT1 protein of the invention. In one embodiment the endogenous DGAT1 polynucleotide has been modified by a gene editing technique.

In a preferred embodiment the cell expresses the modified DGAT1 protein from the poynucleotide or construct.

In one embodiment the modified DGAT1 protein, when expressed in the cell, has at least one of:
i) increased DGAT1 activity,
ii) increased stability, and
iii) altered oligomerisation properties,
   relative to the un-modified DGAT1 when expressed in a cell.

In a further embodiment the cell produces more lipid than does a suitable control cell.

In one embodiment the cell produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid than does a suitable control cell.

### Control Cell

Those skilled in the art will know how to choose a suitable control cell.

In one embodiment the control cell is of the same type, but does not express the modified DGAT1 protein.

In one embodiment the control cell is not transformed with the polynucleotide, or construct, of the invention to express the modified DGAT1 protein.

In one embodiment the control cell is an untransformed cell.

In a further embodiment the control cell is transformed with a control construct. In one embodiment the control construct is an “empty vector” construct. In a further embodiment the control construct expresses the un-modified DGAT1 protein.

In a further embodiment the control cell is a cell that has not been modified, by a gene-editing technique to express the modified DGAT1 protein.

### Cells Also Transformed to Express an Oleosin

In one embodiment the cell is also transformed to express at least one of: an oleosin, steroleosin, caloleosin, polyoleosin, and an oleosin including at least one artificially introduced cysteine (WO2011/053169).

### Plant

In a further embodiment the invention provides a plant comprising a polynucleotide, construct or modified DGAT1 protein of the invention.

In one embodiment the polynucleotide is an transformed into the plant.

In a further embodiment the polynucleotide is an endogenous DGAT1 polynucleotide that has been modified in the plant to encode the modified DGAT1 protein of the invention.

In one embodiment the endogenous DGAT1 polynucleotide has been modified by a gene editing technique.

In a preferred embodiment the plant expresses the modified DGAT1 protein from the poynucleotide or construct.

In one embodiment the modified DGAT1 protein when expressed in the plant has at least one of:
i) increased DGAT1 activity,
ii) increased stability, and
iii) altered oligomerisation properties,
   relative to the unmodified DGAT1.

In a further embodiment the plant produces more lipid, in at least one of its tissues or parts, than does the equivalent tissue or part in a suitable control plant.

In one embodiment the plant produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid, in at least one of its tissues or parts, than does a suitable control plant.

In one embodiment the tissue is a vegetative tissue. In one embodiment the part is a leaf. In a further embodiment the part is a root. In a further embodiment the part is a tuber. In a further embodiment the part is a corm. In a further embodiment the part is a stalk. In a further embodiment the part is a stalk of a monoct plant. In a further embodiment the part is a stovum (stalk and leaf blade).

In a preferred embodiment the tissue is seed tissue. In a preferred embodiment the part is a seed. In a preferred embodiment the part is a cotyledon. In a preferred embodiment the tissue is endosperm tissue.

In a further embodiment the plant as a whole produces more lipid than does the suitable control plant as a whole.

In one embodiment the plant produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid as a whole than does a suitable control plant.

### Plant Also Transformed to Express an Oleosin

In one embodiment the plant is also transformed to express at least one of: an oleosin, steroleosin, caloleosin, polyoleosin, and an oleosin including at least one artificially introduced cysteine (WO 2011/053169).

### Plant Parts

In a further embodiment the invention provides a part, propagule or progeny of a plant of the invention.

In a preferred embodiment the part, propagule or progeny comprises at least one of a polynucleotide, construct or protein of the invention.

In a preferred embodiment the part, propagule or progeny expresses at least one of a polynucleotide, construct or protein of the invention.

In a preferred embodiment the part, propagule or progeny expresses a modified DGAT1 protein of the invention.

In a further embodiment the part, propagule or progeny produces more lipid than does a control part, propagule or progeny, or part, propagule or progeny of a suitable control plant.

In one embodiment the part, propagule or progeny produces at least 5% more, preferably at least 10% more, preferably at least 15% more, preferably at least 20% more, preferably at least 25% more, preferably at least 30% more, preferably at least 35% more, preferably at least 40% more, preferably at least 45% more, preferably at least 50% more, preferably at least 55% more, preferably at least 60% more, preferably at least 65% more, preferably at least 70% more, preferably at least 75% more, preferably at least 80% more, preferably at least 85% more, preferably at least 90% more, preferably at least 95% more preferably at least 100% more, preferably at least 105% more, preferably at least 110% more, preferably at least 115% more, preferably at least 120% more, preferably at least 125% more, preferably at least 130% more, preferably at least 135% more, preferably at least 140% more, preferably at least 145% more, preferably at least 150% more lipid than does a control part, propagule or progeny, or part, propagule or progeny of a suitable control plant.

### Control Plant

Those skilled in the art will no how to choose a suitable control plant.

In one embodiment the control plant is of the same type, and age or developmental stage, but does not express the modified DGAT1 protein.

In one embodiment the control plant is not transformed with the polynucleotide, or construct, of the invention to express the modified DGAT1 protein.

In one embodiment the control plant is an untransformed plant.

In a further embodiment the control plant is transformed with a control construct. In one embodiment the control construct is an “empty vector” construct. In a further embodiment the control construct expresses the un-modified DGAT1 protein.

In a further embodiment the control plant is a plant that has not been modified, by a gene-editing technique to express the modified DGAT1 protein.

Preferably the control part, propagule or progeny is from a control plant as described above.

In one embodiment the part is from a vegetative tissue. In one embodiment the part is a leaf.

In a further embodiment the part is a root. In a further embodiment the part is a tuber. In a further embodiment the part is a corm. In a further embodiment the part is a stalk. In a further embodiment the part is a stalk of a monocot plant. In a further embodiment the part is a stovum (stalk and leaf blade).

In a further embodiment the part is from a reproductive tissue. In a further embodiment the part is a seed. In a preferred embodiment the part is from or includes endosperm tissue.

### Animal Feed

In a further aspect the invention provides an animal feedstock comprising at least one of a polynucleotide, construct, modified DGAT1 protein, cell, plant cell, plant part, propagule and progeny of the invention.

### Biofuel Feedstock

In a further aspect the invention provides a biofuel feedstock comprising at least one of a polynucleotide, construct, modified DGAT1 protein, cell, plant cell, plant part, propagule and progeny of the invention.

### Lipid

In one embodiment the lipid is an oil. In a further embodiment the lipid is triacylglycerol (TAG)

### Method for Producing Oil

In a further aspect the invention provides a method for producing oil, the method comprising extracting lipid from at least one of a cell, plant cell, plant part, propagule and progeny of the invention.

In a further embodiment the lipid is processed into at least one of:
a) a fuel,
b) an oleochemical,
c) a nutritional oil,
d) a cosmetic oil,
e) a polyunsaturated fatty acid (PUFA), and
f) a combination of any of a) to e).

### Method for Increasing the Production of Oil in a Plant

In a further aspect the invention provides a method for increasing the production of oil in a plant, the method comprising the step of expressing the modified DGAT1 protein in the plant.

Those skilled in the art will understand how to express the modified DGAT1 protein in the plant, based on the embodiments described in detail above.

### DETAILED DESCRIPTION OF THE INVENTION

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. In some embodiments, the term “comprising” (and related terms such as “comprise and “comprises”) can be replaced by “consisting of” (and related terms “consist” and “consists”).

### Definitions

The term “DGAT1” as used herein means acyl CoA: diacylglycerol acyltransferase (EC 2.3.1.20)

DGAT1 is typically the major TAG synthesising enzyme in both the seed and senescing leaf (Kaup et al., 2002, Plant Physiol. 129 (4): 1616-26; for reviews see Lung and Weselake 2006, Lipids. December 2006; 41 (12): 1073-88; Cahoon et al., 2007, Current Opinion in Plant Biology. 10:236-244; and Li et al., 2010, Lipids. 45:145-157).

DGAT1 contains approximately 500 amino acids and has been reported to have up to 10 predicted transmembrane domains whereas DGAT2 has only 320 amino acids and is predicted to contain only two transmembrane domains; both proteins were also predicted to have their N- and C-termini located in the cytoplasm (Shockey et al., 2006, Plant Cell 18:2294-2313). Both DGAT1 and DGAT2 have orthologues in animals and fungi and are transmembrane proteins located in the ER.

In most dicotyledonous plants DGAT1 & DGAT2 appear to be single copy genes whereas there are typically two versions of each in the grasses which presumably arose during the duplication of the grass genome (Salse et al., 2008, Plant Cell, 20:11-24).

The term “unmodified DGAT1” as used herein typically means a naturally occurring or native DGAT1. In some cases the DGAT1 sequence may have been assembled from sequences in the genome of a plant, but may not be expressed in the plants.

In one embodiment the un-modified DGAT1 polypeptide sequences have the sequence of any one of SEQ ID NO: 1 to 29 or a variant thereof. Preferably the variant has at least 70% identity to any one of SEQ ID NO: 1 to 29. In a further embodiment the unmodified DGAT1 sequences have the sequence of any one of SEQ ID NO: 1 to 29.

In one embodiment the un-modified DGAT1 polynucleotide sequences have the sequence of any one of SEQ ID NO: 30 to 58 or a variant thereof. Preferably the variant has at least 70% identity to any one of SEQ ID NO: 30 to 58. In a further embodiment the unmodified DGAT1 sequences have the sequence of any one of SEQ ID NO: 30 to 58.

The term “modified DGAT1” as used herein refers to the DGAT1 of the invention that is modified upstream of the N-terminal cytoplasmic acyl-CoA binding site, relative to an unmodified DGAT1. The modification is an alteration in the number or position of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
   where R is arginine, A is Alanine, G is Glycine and X is any amino acid.

In one embodiment the modified DGAT1 protein is at least 90%, preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the un-modified DGAT1 protein.

In one embodiment the modified DGAT1 sequences have the sequence of any SEQ ID NO: 73, 76, 77, 97, 98, 101, 102, 107 and 108 or a variant thereof. Preferably the variant has at least 70% identity to any one of SEQ ID NO: 73, 76, 77, 97, 98, 99, 100, 101, 102, 107 and 108.

In a further embodiment the modified DGAT1 sequences has the sequence of any one of SEQ ID NO: 73, 76, 77, 97, 98, 99, 100, 101, 102, 107 and 108.

In a further embodiment the modified DGAT1 polypeptide sequences have the sequence of any SEQ ID NO: 73, 76, 77, 101, 102, 107 and 108 or a variant thereof. Preferably the variant has at least 70% identity to any one of SEQ ID NO: 73, 76, 77, 101, 102, 107 and 108. In a further embodiment the modified DGAT1 sequence has the sequence of any one of SEQ ID NO: 73, 76, 77, 101, 102, 107 and 108.

In a further embodiment the modified DGAT1 polypeptide sequences have the sequence of any SEQ ID NO: 107 and 108 or a variant thereof. Preferably the variant has at least 70% identity to any one of SEQ ID NO: 107 and 108. In a further embodiment the modified DGAT1 sequence has the sequence of any one of SEQ ID NO: 107 and 108.

Although not preferred, the modified DGAT1 of the invention may include modifications additional to those upstream of the acyl-CoA binding site. Preferably the modified DGAT1 of the invention includes an intact acyl-CoA binding site.

The terms upstream and downstream are according to normal convention to mean towards the N-terminus of a polypeptide, and towards the C-terminus of a polypeptide, respectively.

### Acyl-CoA Binding Site

The position of the acycl-CoA binding site in a number of DGAT1 sequences is shown in FIG. 1.

### Conserved Motif ESPLSS

In a preferred embodiment the acycl-CoA binding site comprises the conserved motif ESPLSS

### Acyl-CoA Binding Site General Formulae

In a preferred embodiment the acyl-CoA binding site has the formula: XXXESPLSSXXIFXXXHA,

where X is any amino acid.

In a preferred embodiment the acyl-CoA binding site has the formula: XXXESPLSSXXIFXXSHA, where X is any amino acid.

In a preferred embodiment the acyl-CoA binding site has the formula: X₁X₂X₃ESPLSSX₄X₅IFX₆X₇X₈HA, where X₁=R, K, V, T, A, S or G; X₂=A, T, V, I, N, R, S or L; X₃=R or K; X₄=D or G; X₅=A, T, N, or L; X₆=K or R; X₇=Q or H; and X₈=S or is absent.

In a preferred embodiment the acyl-CoA binding site has the formula: X₁X₂X₃ESPLSSX₄X₅IFX₆X₇SHA, where X₁=R, K, V, T, A, S or G; X₂=A, T, V, I, N, R, S or L; X₃=R or K; X₄=D or G; X₅=A, T, N, or L; X₆=K or R; and X₇=Q or H.

### Methods for Modifying DGAT1

Methods for modifying the sequence of proteins, or the polynucleotide sequences encoding them, are well known to those skilled in the art. The sequence of a protein may be conveniently be modified by altering/modifying the sequence encoding the protein and expressing the modified protein. Approaches such as site-directed mutagenesis may be applied to modify existing polynucleotide sequences. Altered polynucleotide sequences may also be conveniently synthesised in its modified form.

### Methods for Modifying Endogenous Polynucleotides Gene Editing

Some embodiments of the invention involve modifying and endogenous DGAT1 polynucleotides to express the modified DGAT1 proteins of the invention.

Methods for modifying endogenous genomic DNA sequences in plants are known to those skilled in the art. Such methods may involve the use of sequence-specific nucleases that generate targeted double-stranded DNA breaks in genes of interest. Examples of such methods for use in plants include: zinc finger nucleases (Curtin et al., 2011. Plant Physiol. 156:466-473.; Sander, et al., 2011. Nat. Methods 8:67-69.), transcription activator-like effector nucleases or “TALENs” (Cermak et al., 2011, Nucleic Acids Res. 39: e82; Mahfouz et al., 2011 Proc. Natl. Acad. Sci. USA 108:2623-2628; Li et al., 2012 Nat. Biotechnol. 30:390-392), and LAGLIDADG homing endonucleases, also termed “meganucleases” (Tzfira et a/., 2012. Plant Biotechnol. J. 10:373-389).

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically. A modified version of the CRISPR-Cas9 system has been developed to recruit heterologous domains that can regulate endogenous gene expression or label specific genomic loci in living cells (Sander and Young, Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52).

In certain embodiments of the invention, a genome editing technology (e.g. TALENs, a Zinc finger nuclease or CRISPR-Cas9 technology) can be used to modify one or more base pairs in a target endogenous DGAT1 gene or polynucleotide to create or disrupt a codon encoding an Arginine (R) residue. In this way di-Arginine motifs can be added or removed from the expressed DGAT1 protein in accordance with the invention.

The phrase “increased DGAT1 activity” means increased specific activity relative to that of the un-modified DGAT1.

An art skilled worker would know how to test the “specific activity” of the chimeric DGAT1. This may typically be done by isolating, enriching and quantifying the recombinant DGAT1 then using this material to determine either the rate of triaclyglyceride formation and/or the disappearance of precursor substrates (including various forms of acyl-CoA and DAG) as per Xu et al., (2008), Plant Biotechnology Journal. 6:799-818.

The phrase “increased stability” means that the modified DGAT1 protein is more stable, when expressed in a cell, than the un-modified DGAT1. This may lead to increased accumulation of active modified DGAT1 when it is expressed in cells, releative to when unmodified DGAT1 is expressed in cells.

Those skilled in the art know how to test the “stability” of the modified DGAT1. This would typically involve expressing the modified DGAT1 in a cell, or cells, and expressing the un-modified DGAT1 in a separate cell, or cells of the same type. Accumulation of modified and the un-modified DGAT1 protein in the respective cells can then be measured, for example by immunoblot and/or ELISA. A higher level of accumulation of the modified DGAT1 relative to the un-modified DGAT1, at the same time point, indicates that the modified DGAT1 has increased stability. Alternatively, stability may also be determined by the formation of quaternary structure which can also be determined by immunoblot analysis.

The phrase “altered oligomerisation properties” means that the way in which, or the extent to which modified DGAT1 forms oligomers is altered relative to unmodified DGAT1.

Those skilled in the art know how to test the “oligomerisation properties” of the modified DGAT1. This may typically be done by immunoblot analysis or size exclusion chromatography.

The phrase “substantially normal cellular protein accumulation properties” means that the modified DGAT1 of the invention retains substantially the same protein accumulation when expressed in a cell, as does the unmodified DGAT1. That is there is no less accumulation of modified DGAT1 than there is accumulation of un-modified DGAT1, when either are separately expressed in the same cell type.

An art-skilled worker would know how to test the “cellular protein accumulation properties” of the modified DGAT1. This would typically involve expressing the modified DGAT1 in a cell, or cells, and expressing the unmodified DGAT1 in a separate cell, or cells of the same type. Accumulation of modified and unmodified DGAT1 protein in the respective cells can then be measured, for example by ELISA or immunoblot. A higher level of accumulation of the modified DGAT1 relative to the unmodified DGAT1, at the same time point, indicates that the modified DGAT1 has increased “cellular protein accumulation properties”.

In one embodiment the modified DGAT1 protein has a greater capacity to increase cellular lipid production than does the un-modified DGAT1 protein.

The phrase “greater capacity to increase cellular lipid production” means that the modified DGAT1 of the invention when expressed in a cell increases production of lipid, more than does the unmodified DGAT1.

An art skilled worker would know how to test the “capacity to increase cellular lipid production” of the modifies DGAT1. This would typically involve expressing the modified DGAT1 in a cell, or cells, and expressing the unmodified DGAT1 in a separate cell, or cells of the same type. Lipd production in the respective cells can then be assessed, for example by methods well-known to those skilled in the art and discussed further below. An increase in of lipid production by expression of the modified DGAT1 protein relative to that by the un-modified protein, at the same time point, indicates that the modified DGAT1 protein has a “greater capacity to increase cellular lipid production” than does the unmodified DGAT1 protein.

### Lipid

In one embodiment the lipid is an oil. In a further embodiment the oil is triacylglycerol (TAG)

### Lipid Production

In certain embodiments the cell, tissues, plants and plant parts of the invention produces more lipid than control cells, tissues, plants and plant parts.

Those skilled in the art are well aware of methods for measuring lipid production. This may typically be done by quantitative fatty acid methyl ester gas chromatography mass spectral analysis (FAMES GC-MS). Suitable methods are also described in the examples section of this specification.

### Substrate Specificity

In certain embodiments, the modified DGAT1 proteins of the invention have altered substrate specificity relative to other DGAT1 proteins. Plant DGAT1 proteins are relatively promiscuous in terms of the fatty acid substrates and DAG species they are capable of utilisting to generate TAG. As such they can be considered to have relatively low substrate specificity. However, this can be modified such that certain fatty acids become a preferred substrate over others. This leads to an increase in the proportions of the preferred fatty acids in the TAG and decreases in the proportions of the non-preferred fatty acid species. Substrate specificity can be determined by in vitro quantitiative analysis of TAG production following the addition of specific and known quantities of purified substrates to known quantities of recombinant DGAT, as per Xu et al., (2008), Plant Biotechnology Journal. 6:799-818.

### Cells

The modified DGAT1 of the invention, or as used in the methods of the invention, may be expressed in any cell type.

In one embodiment the cell is a prokaryotic cell. In a further embodiment the cell is a eukaryotic cell. In one embodiment the cell is selected from a bacterial cell, a yeast cell, a fungal cell, an insect cell, algal cell, and a plant cell. In one embodiment the cell is a bacterial cell. In a further embodiment the cell is a yeast cell. In one embodiment the yeast cell is a S. cerevisiae cell. In further embodiment the cell is a fungal cell. In further embodiment the cell is an insect cell. In further embodiment the cell is an algal cell. In a further embodiment the cell is a plant cell.

In one embodiment the cell is a non-plant cell. In one embodiment the non-plant is selected from E. coli, P. pastoris, S. ceriviseae, D. salina, C. reinhardtii. In a further embodiment the non-plant is selected from P. pastoris, S. ceriviseae, D. salina, C. reinhardtii.

In one embodiment the cell is a microbial cell. In another embodiment, the microbial cell is an algal cell of the division of Chlorophyta (green algae), Rhodophyta (red algae), Phaeophyceae (brown algae), Bacillariophycaeae (diatoms), or Dinoflagellata (dinoflagellates). In another embodiment, the microbial cell is an algal cell of the species Chlamydomonas, Dunaliella, Botrycoccus, Chlorella, Crypthecodinium, Gracilaria, Sargassum, Pleurochrysis, Porphyridium, Phaeodactylum, Haematococcus, Isochrysis, Scenedesmus, Monodus, Cyclotella, Nitzschia, or Parietochloris. In another embodiment, the algal cell is Chlamydomonas reinhardtii. In yet another embodiment, the cell is from the genus Yarrowia, Candida, Rhodotorula, Rhodosporidium, Cryptococcus, Trichosporon, Lipomyces, Pythium, Schizochytrium, Thraustochytrium, or Ulkenia. In yet another embodiment, the cell is a bacterium of the genus Rhodococcus, Escherichia, or a cyanobacterium. In yet another embodiment, the cell is a yeast cell. In yet another embodiment, the cell is a synthetic cell.

### Organisms

The term organism includes any organism including animals and plant. Preferably the organism is a plant.

### Plants

The un-modified DGAT1 sequences, from which the modified DGAT1 sequences are produced, may be naturally-occurring DGAT1 sequences. Preferably the unmodified DGAT1 sequences are from plants. In certain embodiments the cells into which the modified DGAT1 proteins are expressed are from plants. In other embodiments the modified DGAT1 proteins are expressed in plants.

The plant cells, from which the modified DGAT1 proteins are derived, the plants from which the plant cells are derived, and the plants in which the modified DGAT1 proteins are expressed may be from any plant species.

In one embodiment the plant cell or plant, is derived from a gymnosperm plant species.

In a further embodiment the plant cell or plant, is derived from an angiosperm plant species.

In a further embodiment the plant cell or plant, is derived from a from dicotyledonous plant species.

In a further embodiment the plant cell or plant, is derived from a monocotyledonous plant species.

Other preferred plants are forage plant species from a group comprising but not limited to the following genera: Zea, Lolium, Hordium, Miscanthus, Saccharum, Festuca, Dactylis, Bromus, Thinopyrum, Trifolium, Medicago, Pheleum, Phalaris, Holcus, Glycine, Lotus, Plantago and Cichorium.

Other preferred plants are leguminous plants. The leguminous plant or part thereof may encompass any plant in the plant family Leguminosae or Fabaceae. For example, the plants may be selected from forage legumes including, alfalfa, clover; leucaena; grain legumes including, beans, lentils, lupins, peas, peanuts, soy bean; bloom legumes including lupin, pharmaceutical or industrial legumes; and fallow or green manure legume species.

A particularly preferred genus is Trifolium. Preferred Trifolium species include Trifolium repens; Trifolium arvense; Trifolium affine; and Trifolium occidentale. A particularly preferred Trifolium species is Trifolium repens.

Another preferred genus is Medicago. Preferred Medicago species include Medicago sativa and Medicago truncatula. A particularly preferred Medicago species is Medicago sativa, commonly known as alfalfa.

Another preferred genus is Glycine. Preferred Glycine species include Glycine max and Glycine wightii (also known as Neonotonia wightii). A particularly preferred Glycine species is Glycine max, commonly known as soy bean. A particularly preferred Glycine species is Glycine wightii, commonly known as perennial soybean.

Another preferred genus is Vigna. A particularly preferred Vigna species is Vigna unguiculata commonly known as cowpea.

Another preferred genus is Mucana. Preferred Mucana species include Mucana pruniens. A particularly preferred Mucana species is Mucana pruniens commonly known as velvetbean.

Another preferred genus is Arachis. A particularly preferred Arachis species is Arachis glabrata commonly known as perennial peanut.

Another preferred genus is Pisum. A preferred Pisum species is Pisum sativum commonly known as pea.

Another preferred genus is Lotus. Preferred Lotus species include Lotus corniculatus, Lotus pedunculatus, Lotus glabar, Lotus temus and Lotus uliginosus. A preferred Lotus species is Lotus corniculatus commonly known as Birdsfoot Trefoil. Another preferred Lotus species is Lotus glabar commonly known as Narrow-leaf Birdsfoot Trefoil. Another preferred preferred Lotus species is Lotus pedunculatus commonly known as Big trefoil. Another preferred Lotus species is Lotus temuis commonly known as Slender trefoil.

Another preferred genus is Brassica. A preferred Brassica species is Brassica oleracea, commonly known as forage kale and cabbage.

Other preferred species are oil seed crops including but not limited to the following genera: Brassica, Carthumus, Helianthus, Zea and Sesamum

A preferred oil seed genera is Brassica. A preferred oil seed species is Brassica napus.

A preferred oil seed genera is Brassica. A preferred oil seed species is Brassica oleraceae.

A preferred oil seed genera is Carthamus. A preferred oil seed species is Carthamus tinctorius.

A preferred oil seed genera is Helianthus. A preferred oil seed species is Helianthus anmus.

A preferred oil seed genera is Zea. A preferred oil seed species is Zea mays.

A preferred oil seed genera is Sesamum. A preferred oil seed species is Sesamum indicum.

A preferred silage genera is Zea. A preferred silage species is Zea mays.

A preferred grain producing genera is Hordeum. A preferred grain producing species is Hordeum vulgare.

A preferred grazing genera is Lolium. A preferred grazing species is Lolium perenne.

A preferred grazing genera is Lolium. A preferred grazing species is Lolium arundinaceum.

A preferred grazing genera is Trifolium. A preferred grazing species is Trifolium repens.

A preferred grazing genera is Hordeum. A preferred grazing species is Hordeum vulgare.

Preferred plants also include forage, or animal feedstock plants. Such plants include but are not limited to the following genera: Miscanthus, Saccharum, Panicum.

A preferred biofuel genera is Miscanthus. A preferred biofuel species is Miscanthus giganteus.

A preferred biofuel genera is Saccharum. A preferred biofuel species is Saccharum officinarum.

A preferred biofuel genera is Panicum. A preferred biofuel speices is Panicum virgatum.

### Plant Parts, Propagues and Progeny

The term “plant” is intended to include a whole plant, any part of a plant, a seed, a fruit, propagules and progeny of a plant.

The term ‘propagule’ means any part of a plant that may be used in reproduction or propagation, either sexual or asexual, including seeds and cuttings.

The plants of the invention may be grown and either self-ed or crossed with a different plant strain and the resulting progeny, comprising the polynucleotides or constructs of the invention, and/or expressing the modified DGAT1 sequences of the invention, also form a part of the present invention.

Preferably the plants, plant parts, propagules and progeny comprise a polynucleotide or construct of the invention, and/or express a modified DGAT1 sequence of the invention.

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence that is complementary to the probe, in a hybridization-based assay. The probe may consist of a “fragment” of a polynucleotide as defined herein.

### Proteins Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides or proteins of the present invention, or used in the methods of the invention, may be purified natural products, or may be produced partially or wholly using recombinant or synthetic techniques. The modified DGAT1 proteins may also be expressed fom endogenous polynucleotides that have been modified using gene editing approaches.

A “fragment” of a polypeptide is a subsequence of the polypeptide that preferably performs a function of and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence of the present invention. Identity is found over a comparison window of at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from the NCBI website on the World Wide Web at ftp://ftp.ncbi.nih.gov/blast/. The default parameters of bl2seq are utilized except that filtering of low complexity parts should be turned off.

The identity of polynucleotide sequences may be examined using the following unix command line parameters:
bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from the world wide web at http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

A preferred method for calculating polynucleotide % sequence identity is based on aligning sequences to be compared using Clustal X (Jeanmougin et al., 1998, Trends Biochem. Sci. 23, 403-5.)

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from the NCBI website on the World Wide Web at ftp://ftp.ncbi.nih.gov/blast/.

The similarity of polynucleotide sequences may be examined using the following unix command line parameters:
bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably less than 1×10-100 when compared with any one of the specifically identified sequences.

Alternatively, variant polynucleotides of the present invention, or used in the methods of the invention, hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C−log (Na+). (Sambrook et al., Eds, 1987, Molecular Cloning, A Laboratory Manual, 2nd Ed. Cold Spring Harbor Press; Bolton and Mccarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.)

With respect to the DNA mimics known as peptide nucleic acids (PNAS) (Nielsen et al., Science. 1991 Dec. 6; 254 (5037): 1497-500) Tm values are higher than those for DNA-DNA or DNA-RNA hybrids, and can be calculated using the formula described in Giesen et al., Nucleic Acids Res. 1998 Nov. 1; 26 (21): 5004-6. Exemplary stringent hybridization conditions for a DNA-PNA hybrid having a length less than 100 bases are 5 to 10° C. below the Tm.

Variant polynucleotides of the present invention, or used in the methods of the invention, also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]) from the NCBI website on the World Wide Web at ftp://ftp.ncbi.nih.gov/blast/via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from the NCBI website on the World Wide Web at ftp://ftp.ncbi.nih.gov/blast/. The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5.)

Polypeptide variants of the present invention, or used in the methods of the invention, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from the NCBI website on the World Wide Web at ftp://ftp.ncbi.nih.gov/blast/. The similarity of polypeptide sequences may be examined using the following unix command line parameters:
bl2seq -i peptideseq1 -j peptideseq2 -F F -p blastp

Variant polypeptide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably 1×10-100 when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence may, in some cases, identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination, mRNA stability, and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors. Introns within coding sequences can also regulate transcription and influence post-transcriptional processing (including splicing, capping and polyadenylation).

A promoter may be homologous with respect to the polynucleotide to be expressed. This means that the promoter and polynucleotide are found operably linked in nature.

Alternatively the promoter may be heterologous with respect to the polynucleotide to be expressed. This means that the promoter and the polynucleotide are not found operably linked in nature.

In certain embodiments the modified DGAT1 polynucleotides/polypeptides of the invention may be advantageously expessed under the contol of selected promoter sequences as described below.

### Vegetative Tissue Specific Promoters

An example of a vegetative specific promoter is found in U.S. Pat. Nos. 6,229,067; and 7,629,454; and 7,153,953; and 6,228,643.

### Pollen Specific Promoters

An example of a pollen specific promoter is found in U.S. Pat. Nos. 7,141,424; and 5,545,546; and 5,412,085; and 5,086,169; and 7,667,097.

### Seed Specific Promoters

An example of a seed specific promoter is found in U.S. Pat. Nos. 6,342,657; and 7,081,565; and 7,405,345; and 7,642,346; and 7,371,928. A preferred seed specific promoter is the napin promoter of Brassica napus (Josefsson et al., 1987, J Biol Chem. 262 (25): 12196-201; Ellerström et al., 1996, Plant Molecular Biology, Volume 32, Issue 6, pp 1019-1027).

### Fruit Specific Promoters

An example of a fruit specific promoter is found in U.S. Pat. Nos. 5,536,653; and 6,127,179; and 5,608,150; and 4,943,674.

### Non-Photosynthetic Tissue Preferred Promoters

Non-photosynthetic tissue preferred promoters include those preferentially expressed in non-photosynthetic tissues/organs of the plant.

Non-photosynthetic tissue preferred promoters may also include light repressed promoters.

### Light Repressed Promoters

An example of a light repressed promoter is found in U.S. Pat. No. 5,639,952 and in U.S. Pat. No. 5,656,496.

### Root Specific Promoters

An example of a root specific promoter is found in U.S. Pat. No. 5,837,848; and US 2004/0067506 and US 2001/0047525.

### Tuber Specific Promoters

An example of a tuber specific promoter is found in U.S. Pat. No. 6,184,443.

### Bulb Specific Promoters

An example of a bulb specific promoter is found in Smeets et al., (1997) Plant Physiol. 113:765-771.

### Rhizome Preferred Promoters

An example of a rhizome preferred promoter is found Seong Jang et al., (2006) Plant Physiol. 142:1148-1159.

### Endosperm Specific Promoters

An example of an endosperm specific promoter is found in U.S. Pat. No. 7,745,697.

### Corm Promoters

An example of a promoter capable of driving expression in a corm is found in Schenk et al., (2001) Plant Molecular Biology, 47:399-412.

### Photosythetic Tissue Preferred Promoters

Photosythetic tissue preferred promoters include those that are preferrentially expressed in photosynthetic tissues of the plants. Photosynthetic tissues of the plant include leaves, stems, shoots and above ground parts of the plant. Photosythetic tissue preferred promoters include light regulated promoters.

### Light Regulated Promoters

Numerous light regulated promoters are known to those skilled in the art and include for example chlorophyll a/b (Cab) binding protein promoters and Rubisco Small Subunit (SSU) promoters. An example of a light regulated promoter is found in U.S. Pat. No. 5,750,385. Light regulated in this context means light inducible or light induced.

A “transgene” is a polynucleotide that is taken from one organism and introduced into a different organism by transformation. The transgene may be derived from the same species or from a different species as the species of the organism into which the transgene is introduced.

### Host Cells

Host cells may be derived from, for example, bacterial, fungal, yeast, insect, mammalian, algal or plant organisms. Host cells may also be synthetic cells. Preferred host cells are eukaryotic cells. A particularly preferred host cell is a plant cell, particularly a plant cell in a vegetative tissue of a plant.

A “transgenic plant” refers to a plant which contains new genetic material as a result of genetic manipulation or transformation. The new genetic material may be derived from a plant of the same species as the resulting transgenic plant or from a different species.

### Methods for Isolating or Producing Polynucleotides

The polynucleotide molecules of the invention can be isolated by using a variety of techniques known to those of ordinary skill in the art. By way of example, such polypeptides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides of the invention include use of all, or portions of, the polypeptides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion, oligonucleotide synthesis and PCR amplification.

A partial polynucleotide sequence may be used, in methods well-known in the art to identify the corresponding full length polynucleotide sequence. Such methods include PCR-based methods, 5′RACE (Frohman M A, 1993, Methods Enzymol. 218:340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. For these reasons among others, it is desirable to be able to identify and isolate orthologues of a particular gene in several different plant species.

Variants (including orthologues) may be identified by the methods described.

### Methods for Identifying Variants

### Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules of the invention by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

The variant sequences of the invention, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29:1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, MD 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25:3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302:205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the invention, or used in the methods of the invention, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco California, or automated synthesis, for example using an Applied Biosystems 431A Peptide Synthesizer (Foster City, California). Mutated forms of the polypeptides may also be produced during such syntheses.

The polypeptides and variant polypeptides of the invention, or used in the methods of the invention, may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides of the invention, or used in the methods of the invention, may be expressed recombinantly in suitable host cells and separated from the cells as discussed below.

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

### Methods for Producing Host Cells Comprising Polynucleotides, Constructs or Vectors

The invention provides a host cell which comprises a genetic construct or vector of the invention.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention, or used in the methods of the invention. Plants comprising such cells also form an aspect of the invention.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of plant transformation strategies are available (e.g. Birch, 1997, Ann Rev Plant Phys Plant Mol Biol, 48, 297; Hellens et al., 2000, Plant Mol Biol 42:819-32; Hellens et al., Plant Meth 1:13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues respond to internal developmental signals or external abiotic or biotic stresses are described in the scientific literature. Exemplary promoters are described, e.g., in WO 02/00894 and WO2011/053169, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9: 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); Brassica (U.S. Pat. Nos. 5,188,958; 5,463,174 and 5,750,871); cereals (U.S. Pat. No. 6,074,877); pear (Matsuda et al., 2005, Plant Cell Rep. 24 (1): 45-51); Prunus (Ramesh et al., 2006 Plant Cell Rep. 25 (8): 821-8; Song and Sink 2005 Plant Cell Rep. 2006; 25 (2): 117-23; Gonzalez Padilla et al., 2003 Plant Cell Rep.22 (1): 38-45); strawberry (Oosumi et al., 2006 Planta. 223 (6): 1219-30; Folta et al., 2006 Planta April 14; PMID: 16614818), rose (Li et al., 2003), Rubus (Graham et al., 1995 Methods Mol Biol. 1995; 44:129-33), tomato (Dan et al., 2006, Plant Cell Reports V25: 432-441), apple (Yao et al., 1995, Plant Cell Rep. 14, 407-412), Canola (Brassica napus L.). (Cardoza and Stewart, 2006 Methods Mol Biol. 343:257-66), safflower (Orlikowska et al, 1995, Plant Cell Tissue and Organ Culture 40:85-91), ryegrass (Altpeter et al., 2004 Developments in Plant Breeding 11 (7): 255-250), rice (Christou et al., 1991 Nature Biotech. 9:957-962), maize (Wang et al., 2009 In: Handbook of Maize pp. 609-639) and Actinidia eriantha (Wang et al., 2006, Plant Cell Rep. 25,5:425-31). Transformation of other species is also contemplated by the invention. Suitable methods and protocols are available in the scientific literature.

### Modification of Endogenous Genomes

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of cell types and in organisms that have traditionally been challenging to manipulate genetically. A modified version of the CRISPR-Cas9 system has been developed to recruit heterologous domains that can regulate endogenous gene expression or label specific genomic loci in living cells (Nature Biotechnology 32, 347-355 (2014). The system is applicable to plants, and can be used to regulate expression of target genes. (Bortesi and Fischer, Biotechnology Advances Volume 33, Issue 1, January-February 2015, Pages 41-52). Use of CRISPR technology in plants is also reviewed in Zhang et al., 2019, Nature Plants, Volume 5, pages 778-794.

### BRIEF DESCRIPTION OF THE FIGURES

FIG. 1 shows the alignment of peptide sequences of the N-terminal cytoplasmic region of a number of plant DGAT1s including both long and short versions from the grasses as well as examples from dicotyledonous species. Left hand box represents acyl-CoA binding site (Nykiforuk et al., 2002, Biochimica et Biophysica Acta 1580:95-109). Right hand box represents first transmembrane region (McFie et al., 2010, JBC., 285:37377-37387). Left hand arrow represents boundary between exon 1 and exon 2. Right hand arrow represents boundary between exon 2 and exon 3. The sequences are AtDGAT1 (SEQ ID NO:113), BjDGAT1 (SEQ ID NO:114), BnDGAT1-AF (SEQ ID NO:115), BjDGAT1 (SEQ ID NO:116), TmajusDGAT1 (SEQ ID NO:117), EpDGAT1 (SEQ ID NO:118), VgDGAT1 (SEQ ID NO: 119), NtDGAT1 (SEQ ID NO:120), PfDGAT1 (SEQ ID NO:121), ZmL (SEQ ID NO: 122), SbDGAT1 (SEQ ID NO:123), OsL (SEQ ID NO:124), OsS (SEQ ID NO:125), SbDGAT1 (SEQ ID NO:126), ZmS (SEQ ID NO:127), PpDGAT1 (SEQ ID NO:128), SmDGAT1 (SEQ ID NO:129), EaDGAT1 (SEQ ID NO:130), VVDGAT1 (SEQ ID NO:131), GmDGAT1 (SEQ ID NO:132), GmDGAT1 (SEQ ID NO:133), LjDGAT1 (SEQ ID NO:134), MtDGAT1 (SEQ ID NO:135), JcDGAT1 (SEQ ID NO:136), VIDGAT1 (SEQ ID NO:137), RcDGAT1 (SEQ ID NO:138), PtDGAT1 (SEQ ID NO:139), Pt DGAT1 (SEQ ID NO:140).

FIG. 2 shows alignment of N-terminal peptide sequences of Arabidopsis thaliana, At (SEQ ID NO: 1); Tropolium majus, Tm (SEQ ID NO:5); Zea mays, ZmL (SEQ ID NO:10); Zea mays, ZmS (SEQ ID NO:15). Underlined type denotes the beginning of the conserved region (WO/2014/068439) which starts 13 residues upstream of the acetyl-CoA binding domain (not shown). Each arginine residue associated with a potential di-arginine motif is shown by bold face type, while the complete AXXXA and GXXXG motifs are shown by bold face underline type.

FIG. 3 shows alignments of N-terminal peptide sequence from ZmL (SEQ ID NO:10) with versions containing modified di-argine motifs. Underlined type denotes the beginning of the conserved region (WO/2014/068439) which starts 13 residues upstream of the acetyl-CoA binding domain (not shown). Each arginine residue associated with a potential di-arginine motif is shown by bold face type. Di-arginine motifs replaced by serine or glycine residues (shown by bold face and underlined S or G respectively) create ZmL^{R13G,R14S,R17S,R65G,R66G} (SEQ ID NO:97) and ZmL^{R33S,R34S,R37S,R65G,R66G} (SEQ ID NO:98).

FIG. 4 shows alignments of N-terminal peptide sequences from Tm (SEQ ID NO:94) with two versions having internal deletions. Underlined type denotes the beginning of the conserved region (WO/2014/068439) which starts 13 residues upstream of the acetyl-CoA binding domain (not shown). Two versions of Tm with internal deletions were made; each had a separate internal section of 27 residues deleted (shown within blocks); in each case this reduced the length of the cytoplasmic N-terminus to be the same as ZmS. One removed the last 27 residues before the conserved region; this also removed the three AXXXA/GAAAG (shown by bold face and underline) motifs creating Tm^{Δ68-94} (SEQ ID NO:99). The second removed a segment closer to the N-terminus; this placed the multiple AXXXA/GAAAG motifs (shown by bold face and underline) closer to the multi di-argine motif (bold face arginine resides) creating Tm^{Δ35-61} (SEQ ID NO:100).

FIG. 5 shows alignments of N-terminal peptide sequences from the chimeras Tm::ZmL (SEQ ID NO: 111) and ZmS::Tm (SEQ ID NO:112) with the same chimeras that had their di-arginine motifs deleted. Underlined type denotes the beginning of the conserved region (WO/2014/068439) which starts 13 residues upstream of the acetyl-CoA binding domain (not shown). Each arginine residue associated with a potential di-arginine motif is shown by bold face type. Deletion of the multi-diargine motif (shown within blocks) from the chimera Tm::ZmL created Tm^{ΔR25,R26,R27}::ZmL (SEQ ID NO:103). Deletion of the multi-diargine motif (shown within blocks) from the chimera ZmS::Tm created ZmS^{ΔR27,L28,R29,R30}::Tm (SEQ ID NO:104).

FIG. 6 shows alignments of N-terminal peptide sequences from the chimeras Tm::ZmL (SEQ ID NO:111) and ZmS::Tm (SEQ ID NO:112) with the same chimeras that had their AXXXA and GXXXG motifs perturbed by substitution. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. Substitution of one alanine and one glycine residue (shown by blocks) with serine residues created Tm^{A64S,G80S}::ZmL (SEQ ID NO:101). Substitution of two alanine and two glycine residues with serine residues created ZmS^{A10S,G17S,A36S,G37S}::Tm (SEQ ID NO:102).

FIG. 7 shows alignment of N-terminal peptide sequences from the chimera Tm::ZmL (SEQ ID NO: 111) with the same chimera that had the AXXXA and GXXXG motifs perturbed by substitution and in one case replaced with a multi di-arginine motifs. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. Substitution of one alanine and three glycine residue (shown by blocks) with serine residues created Tm^{A64S,G78R,G79R,G80R}::ZmL (SEQ ID NO:105).

FIG. 8 shows alignment of N-terminal peptide sequences from the chimera ZmS::Tm (SEQ ID NO: 112) with the same chimera that had a AXXXA motif perturbed and replaced with a multi di-arginine motifs by substitution and an additional multi di-arginine motif created by substitution. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. Substitution of one aspartate and one alanine (shown by blocks) with arginine residues perturbed the N-terminal AXXXA motif with a multi di-arginine motif and substitution of one serine and two glycine residues (shown by blocks) generated an additional multi di-arginine motif creating ZmS^{D12R,A14R,S44R,G45R,G46R}::Tm (SEQ ID NO:106).

FIG. 9 shows alignment of N-terminal peptide sequences from ZmS (sequence ID number 96) and Tm (SEQ ID NO:94) with the same sequences where an additional N-terminal multi di-arginine motif had been created by substitution. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. The substituted residues are shown by blocks; this created ZmS^{A9R,A10R,S11R} (SEQ ID NO:107) and Tm^{S6R,S7R,Q8R} (SEQ ID NO:108).

FIG. 10 shows alignment of N-terminal peptide sequence from At (sequence ID number 75) with the same sequences multiple AXXXA and GXXXG motifs were perturbed by substitution. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. The substituted residues are shown by blocks; this created At^{A75S,G79S,G97S,G99S} (SEQ ID NO:76).

FIG. 11 shows alignment of N-terminal peptide sequence from At (SEQ ID NO:75) with the same sequences multiple AXXXA and GXXXG motifs were perturbed and an additional multi di-arginine motifs was created by substitution. Underlined type denotes the beginning of the conserved region (WO/2014/068439) which starts 13 residues upstream of the acetyl-CoA binding domain (not shown). Each arginine residue associated with a potential di-arginine motif is shown by bold face type. The AXXXA/GAAAG motifs are shown by bold face and underline. The substituted residues are shown by blocks; this created At^{A75S,G79S,G93R,G94R,G95R,G97S,G99S} (SEQ ID NO:77).

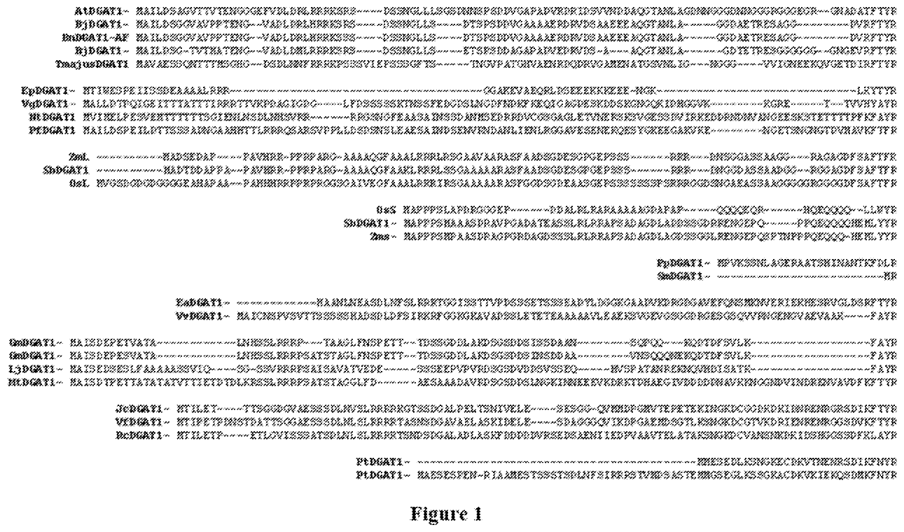

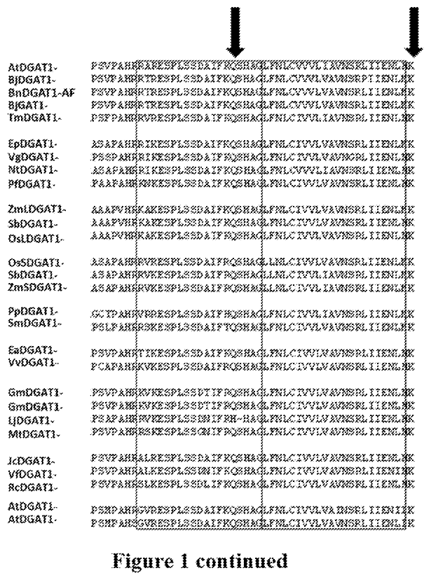

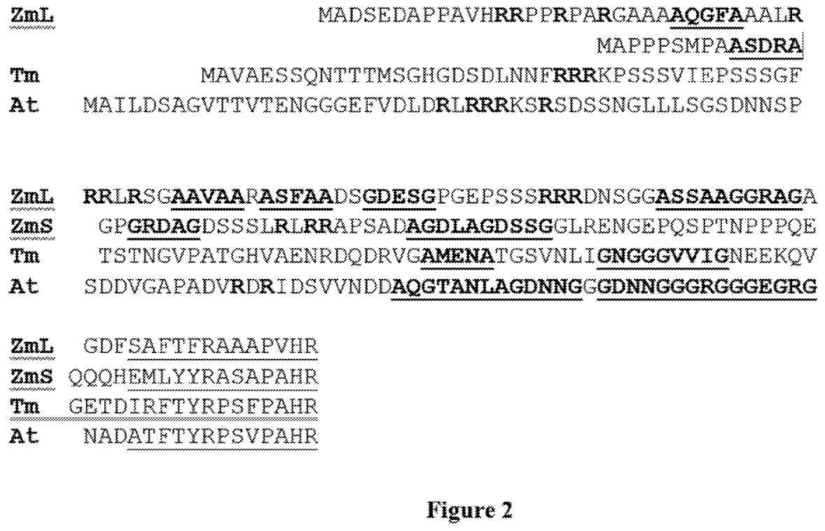

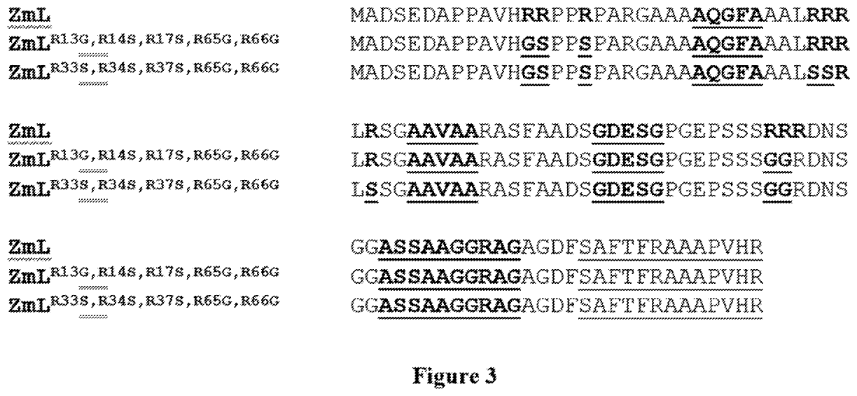

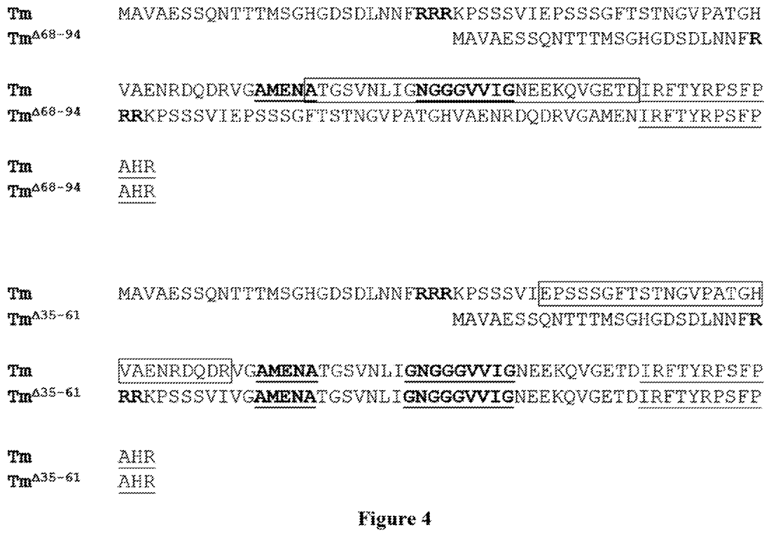

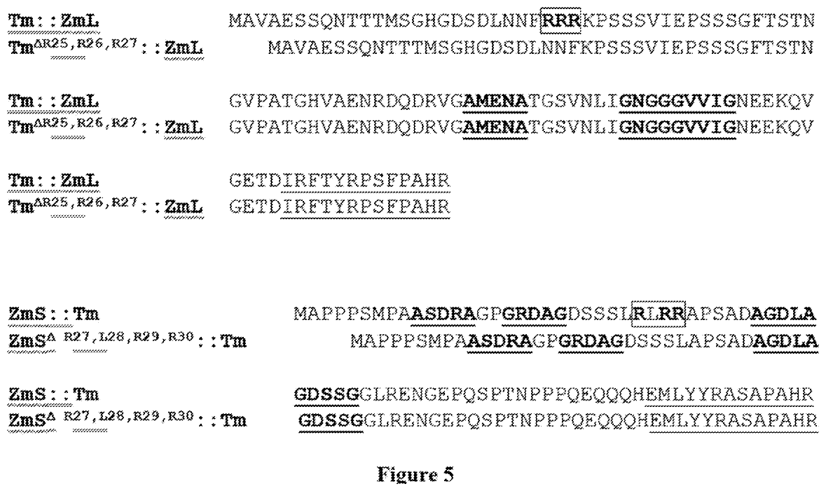

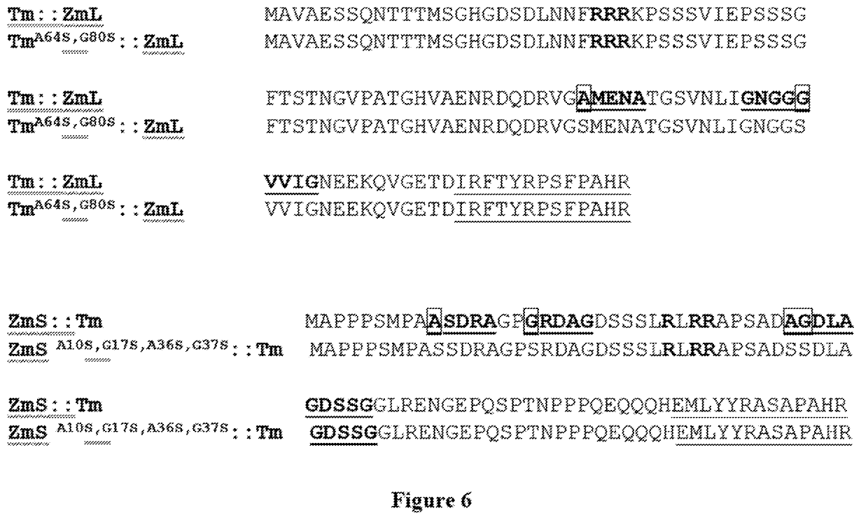

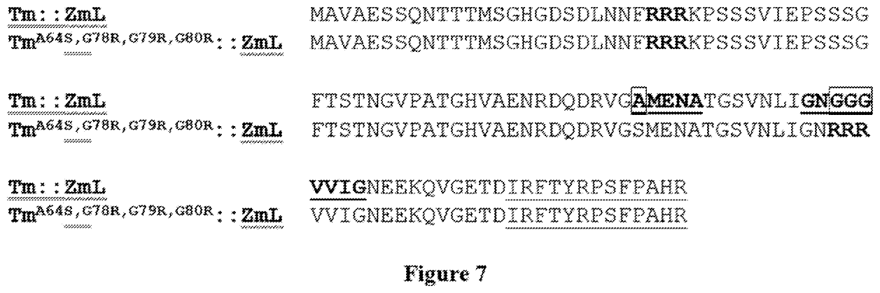

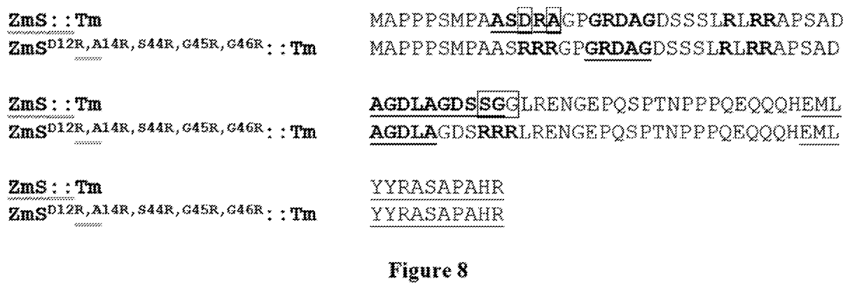

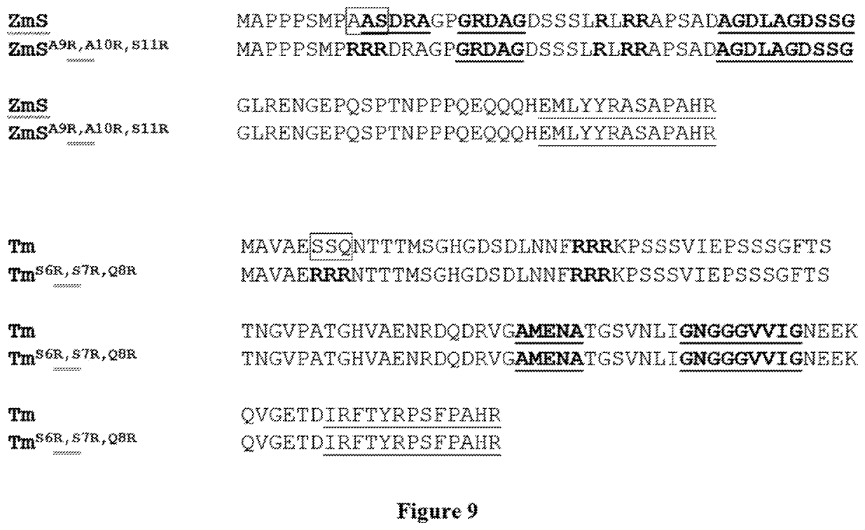

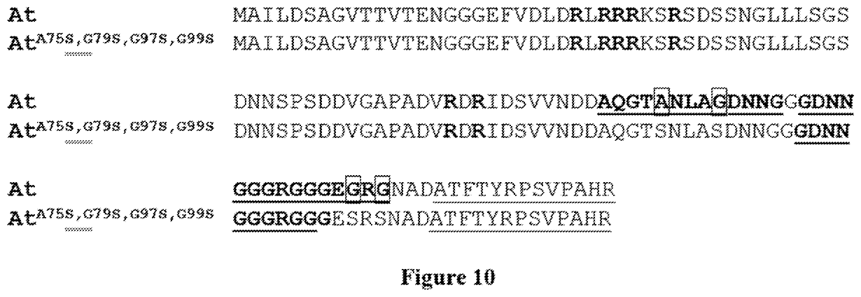

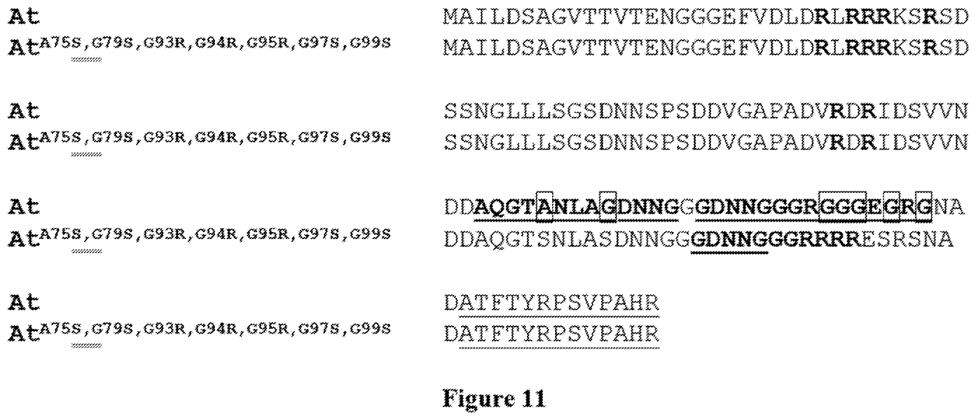

### EXAMPLES

The invention will now be described with reference to the following non-limiting examples.

### Example 1: Plant DGAT1 Contain Multiple N-Terminal Di-Arginine Motifs as Well as AXXXA, GXXXG, AXXXG and GXXXA Motifs

DGAT1s from a broad range of organisms were found to contain a cluster of arginines in the first 30 residues (Siloto et al 2010). Upon examination we found most vascular plant DGAT1s from Table 1 contain multiple di-arginine motifs (RR, RXR, and RXXR) in the variable region of the N-terminus (Table 2). In comparison, the N-terminus of the mammalian DGAT1s (including: Bos Taurus, NP_777118; Mus musculus, NP_034176; Homo sapiens, NP_036211; Ovis aries, NP_0011036; Rattus norvegicus, NP_445889; Sus scrofa, NP_999216; and Mesocricetus auratus, XP_005086048) all contain the same multi motif (RRRR) close to the N-terminus and a second potential motif (RXXR) at the start of the acyl CoA binding region.

In other proteins the di-arginine motifs appear to have roles in assembly of heteromultimeric membrane proteins; retrieval of ER membrane proteins from the Golgi apparatus and the ER-Golgi intermediate; establishing the cytosolic location of the N-terminus; interaction with downstream cytosolic loops (Boulaflous et al 2009; Michelsen et al, 2005; Parks and Lamb 1993; Shikano and Li, 2003; Teasdale and Jackson 1996).

The cytosolic N-termini of the plant DGAT1s also contain varying numbers of AXXXA and GXXXG (includes AXXXG and GXXXA) motifs (Table 3); these have been shown to be involved in protein-protein interaction in transmembrane domains and in cytosolic proteins of thermophilic organisms (Teese and Langosch 2015; Kleiger et al 2002). As such the applicants postulate that they could potentially be involved in oligomerization of DGAT1.

TABLE 1

| DGAT1 | PROTEIN | | | |
|---|---|---|---|---|
| Species | accession #s | SEQ ID | DNA accession #s | SEQ ID |
| Source | & BAC # | NO: | & BAC # | NO: |
| A. thaliana | NP 179535 | 1 | NM 127503 | 30 |
| B. juncea | AAY40784 | 2 | AF164434 | 31 |
| B. napus | AAD45536.1 | 3 | AF164434 1 | 32 |
| B. juncea | AAY40785 | 4 | DQ016107 | 33 |
| T. majus | AAM03340 | 5 | AY084052 | 34 |
| E. pitardii | ACO55635 | 6 | FJ226588 | 35 |
| V. galamensis | ABV21945 | 7 | EF653276 | 36 |
| N. tabacum | AAF19345.1 | 8 | AF129003_1 | 37 |
| P. frutescens | AAG23696.1 | 9 | AF298815 1 | 38 |
| Z. mays | From: CHORI-201 | 10 | From: CHORI-201 | 39 |
| | Maize B73 BAC | | Maize B73 BAC | |
| S. bicolor | XP 002439419 | 11 | XM 002439374 | 40 |
| O. sativa | NP_001054869 | 12 | Os05g0196800 | 41 |
| O. sativa | From: AP003714.1 | 13 | From: AP003714.1 | 42 |
| S. bicolor | XP 002437165 | 14 | XM 002437120.1 | 43 |
| | ABV91586 | 15 | EU039830 | 44 |
| P. patens | XP_001770929 | 16 | XM_001770877.1 | 45 |
| S. moellendorffii | XP_002964165 | 17 | XM_002964119 | 46 |
| E. alatus | AAV31083 | 18 | AY751297 | 47 |
| V. vinifera | XP 002279345 | 19 | XM 002279309 | 48 |
| G. max | AAS78662 | 20 | AY496439 | 49 |
| G. max | BAE93461 | 21 | AB257590 | 50 |
| L. japonicus | AAW51456 | 22 | AY859489 | 51 |
| M. truncatula | ABN09107 | 23 | AC174465.2 | 52 |
| J. curcas | ABB84383 | 24 | DQ278448.1 | 53 |
| V. fordii | ABC94472 | 25 | DQ356680.1 | 54 |
| V. galamensis | ABV21945 | 26 | EF653276.1 | 55 |
| R. communis | XP_002514132 | 27 | XM_002514086.1 | 56 |
| P. trichocarpa | XP 002308278 | 28 | XM 002308242.1 | 57 |
| P. trichocarpa | XP 002330510 | 29 | XM 002330474.1 | 58 |

TABLE 2

| | | Position of | Position of | Position of | |
|---|---|---|---|---|---|
| | | first N- | first N- | first N- | Number of |
| | | terminally | terminally | terminally | di-arginine |
| | | located | located | located | clusters |
| | | arginine | arginine | arginine | within the |
| DGAT1 | | residue of di- | residue of di- | residue of di- | first 65 N- |
| Species | SEQ | arginine motif | arginine motif | arginine motif | terminal |
| Source | ID NO: | RR | RXR | RXXR | residues |
| A. thaliana | 1 | 27, 28, 115 | 116 | 25 29, 115 | 1 |
| B. juncea | 2 | 26, 98 | 54, 99 | 23, 27, 98 | 2 |
| B. napus | 3 | 26, 98 | 54, 99 | 23, 98 | 2 |
| B. juncea | 4 | 24, 25, 98 | 24, 99 | 26, 98 | 1 |
| T. majus | 5 | 25, 26, 107 | 25, 108 | 107 | 1 |
| E. pitardii | 6 | 20, 21, 60 | 20 | | 2 |
| V. galamensis | 7 | 21, 22, 110 | 21 | | 1 |
| N. tabacum | 8 | 33, 34, 35, 58, | 33, 34 | 33 | 2 |
| | | 123 | | | |
| P. frutescens | 9 | 28, 29 | 28 | | 1 |
| Z. mays L | 10 | 3, 33, 34, 65, 66 | 33, 35, 63 | 14, 17, 34 | 3 |
| S. bicolor L | 11 | 5, 35, 36. 67, 68 | 35, 67 | 16, 19 | 2 |
| O. sativa L | 12 | 6, 47, 48, 85, 86 | 30, 47, 49, 85 | 27, 48 | 2 |
| O. sativa S | 13 | 62 | 21, 23, 63 | 62 | 2 |
| S. bicolor S | 14 | 29, 47, 76 | 27 | 27 | 2 |
| Z. mays S | 15 | 29, 80 | 27 | 27 | 1 |
| P. patens | 16 | 36 | 34 | 34 | 1 |
| S. moellendorffii | 17 | 9 | | | 1 |
| E. alatus | 18 | 16, 17 | 16 | | 1 |
| V. vinifera | 19 | 28 | | | 1 |
| G. max | 20 | 20, 21 | 20 | | 1 |
| G. max | 21 | 20, 21 | 20 | | 1 |
| L. japonicus | 22 | 27, 28, 98 | 27 | | 1 |
| M. truncatula | 23 | 32, 33, 127 | 32 | | 1 |
| J. curcas | 24 | 26, 27, 28 | 26, 27, 88 | 26, 85, 105 | 1 |
| V. fordii | 25 | 30, 31, 32 | 30, 31 | 30, 90 | 1 |
| V. galamensis | 26 | 21, 22, 110 | 21 | | 1 |
| R. communis | 27 | 26, 27, 28 | 26, 27 | 26 | 1 |
| P. trichocarpa | 28 | 34, 35 | 34 | 84 | 1 |
| P. trichocarpa | 29 | 29, 30 | 29 | | 1 |

TABLE 3

| | SEQ | |
|---|---|---|
| DGAT1 | ID | Position of first N-terminal A or G residue located in a |
| Species Source | NO: | AXXXA, AXXXG, GXXXG or GXXXA motif |
| A. thaliana | 1 | 71, 75, 79, 85, 89, 90, 91, 95, 97, 99 |
| B. juncea | 2 | 48, 61, 65, 69, 72, |
| B. napus | 3 | 63, 75, 76, |
| B. juncea | 4 | 48, 61, 65, 69, 72, |
| T. majus | 5 | 64, 76, 80, |
| E. pitardii | 6 | 25, 54 |
| V. galamensis | 7 | 31 |
| N. tabacum | 8 | 40, 63, 117 |
| P. frutescens | 9 | 17, 85 |
| Z. mays L | 10 | 19, 21, 23, 25, 27, 39, 40, 46, 49, 53, 72, 73, 77, 78, 79, 84 |
| S. bicolor L | 11 | 21, 23, 25, 27, 41, 42, 44, 48, 51, 55, 75, 78, 81, 82, 87 |
| O. sativa L | 12 | 6, 8, 11, 13, 15, 33, 37, 41, 53, 54, 56, |
| | | 63, 67, 89, 96, 99, 100, 101, 103, 104, 105, 110 |
| O. sativa S | 13 | 8, 24, 26, 27, 29, 31, 56 |
| S. bicolor S | 14 | 10, 14, 36, 70 |
| Z. mays S | 15 | 10, 17, 36, 37, 41, 74 |
| P. patens | 16 | 9, 28 |
| S. moellendorffii | 17 | 0 |
| E. alatus | 18 | 44, 45 |
| V. vinifera | 19 | 33, 49, 53, 67, 86, 89 |
| G. max | 20 | 40, 44 |
| G. max | 21 | 25, 43, 47 |
| L. japonicus | 22 | 12, 85 |
| M. truncatula | 23 | 37, 43, 47, 50, |
| J. curcas | 24 | 11, 73 |
| V. fordii | 25 | 13, 77 |
| V. galamensis | 26 | 31 |
| R. communis | 27 | 0 |
| P. trichocarpa | 28 | 15 |
| P. trichocarpa | 29 | 0 |

### Example 2: Generation of Recombinant Constructs for Evaluation in Saccharomyces cerevisiae

A series of construct where the di-arginine and/or AXXXA, GXXXG motifs were altered/introduced. The name of the constructs, a description of their derivation, and the corresponding peptide sequences are shown in Table 4. All DGAT1s were optimised for expression in Saccharomyces cerevisiae and had an in-frame C-terminal V5 epitope and 6× histidine tag.

Saccharomyces cerevisiae optimized DGAT1 coding sequences along with a C-terminus V5-His tag were synthesised by either GeneArt (Thermo Fisher Scientific) or GenScript and subsequently cloned into the pYES2.1/V5-His-TOPO yeast expression vector (Life Technologies, K4150-01) as per the manufacturer's instructions. This places all DGAT1s expressed in yeast were under the control of the inducible Gall promoter.

TABLE 4

| | | | | NUCLEIC | |
|---|---|---|---|---|---|
| Original | | | | ACID SEQ | |
| Source of | | | | OPTIMIZED | EXPRESSED |
| DGAT | | | | FOR YEAST | PROTEIN |
| Sequence | Name | Description | FIG. | SEQ ID NO: | SEQ ID NO: |
| T. majus | Tm | DGAT1 from T. majus | 1, 2, | 59 | 69 |
| Z. mays | ZmL | Long DGAT1 from Z. mays | 1, 2, | 60 | 70 |
| T. majus & | Tm::ZmL | Chimeric DGAT1 using Tm N- | 5, 6, 7 | 61 | 71 |
| Z. mays | | terminus and ZmL C-terminus | | | |
| Z. mays | ΔN ZmL | ZmL with tr [symbol] ted N-terminus | N/A | 62 | 72 |
| T. majus | Tm [symbol] | Tm Di-arginine motifs | N/A | 63 | 73 |
| | | substituted | | | |
| T. majus and | Tm [symbol] ::ZmL | Tm Di-arginine motifs | N/A | 64 | 74 |
| Z. mays | | substituted and chimera | | | |
| | | generated with ZmL C-terminus | | | |
| A. thaliana | At | DGAT1 from A. thaliana | 1, 2, 10, | 65 | 75 |
| | | | 11 | | |
| A. thaliana | At [symbol] | At with multiple disrupted | 10 | 66 | 76 |
| | | AXXXA and GXXXG | | | |
| A. thaliana | At [symbol] | At with multiple disrupted | 11 | 67 | 77 |
| | | AXXXA and GXXXG and new | | | |
| | | RRRR | | | |
| A. thaliana | ΔN At | At with truncated N-terminus | N/A | 68 | 78 |

[symbol]

indicates data missing or illegible when filed

### Example 3: Generation of Recombinant Constructs for Evaluation in Camelina sativa

A series of construct where the di-arginine and/or AXXXA, GXXXG motifs were altered/introduced. The name of the constructs, a description of their derivation, and the corresponding peptide sequences are shown in Table 5. All DGAT1s had an Arabidopsis thaliana DGAT1 intron 3 (Accession NC_003071, REGION: 8426117 . . . 8429853); the constructs were optimised for expression in Camelina sativa and had an in-frame C-terminal V5 epitope and 6× histidine tag. In addition, the putative serine/threonine protein kinase site in the Tropaeolum majus DGAT1 (Xu et al., 2008) was disrupted by substitution of the serine to alanine generating Tm^{S197A}, Tm^{Δ68-94,S197A}, Tm^{Δ35-61,S197A}, ZmS^{A10S,G17S,A36S,G37S}::Tm^{S170A}, ZmS^{ΔR27,1.28,R29,R30}::Tm^{S170A}, ZmS^{D12R,A14R,S44R,G45R,G46R}::Tm^{S170A}, Tm^{S6R,S7R,Q8R,S197A}, ZmS::Tm^{S170A}.

Brassica optimized DGAT1 coding sequences along with a C-terminus V5-His tag were synthesised by either GeneArt (Thermo Fisher Scientific) or GenScript and sub cloned into pDONR™221. A cassette consisting of Not I sites flanking the Brassica napus napin seed storage promoter region and 5′UTR (GenBank accession number EF627523.1)::GATEWAY® cloning sequences::octopine synthase terminator was synthesised by GenScript. The cassette was digested with Not I and cloned into pRSh1 (Scott et al 2010) replacing the constitutive promoter cauliflower mosaic virus 35S (CaMV35Sp) driven GATEWAY® adapted expression cassette. This created the binary vector pBR2 (ref from Somrutai) containing a seed specific expression cassette in a back-to back orientation with the CaMV35Sp driven bar gene for phosphinothricin resistant selection. DGAT1s were subsequently placed into pBR2 from pDONR™221 by GATEWAY® LR cloning (Thermo Fisher Scientific).

Camelina sativa Transformation

C. sativa (cf. Calena) were transformed via Agrobacterium tumefaciens (GV3101) using the floral dip method (adapted from that of Clough and Bent, 1998, Plant J. 16 (6): 735-745). Essentially seeds were sown in potting mix in 10 cm pots in a controlled environment, approximately 6 weeks after planting the flowers were dipped for 5-14 minutes under vacuum (70-80 mm Hg) in an overnight culture of appropriated Agrobacterium GV3101 cells re-suspended in a floral dip buffer. After vacuum-transformation, plants were kept for 24 h under low light conditions by partly covering with a black plastic sheet. Vacuum transformations can be repeated three times at approximately 10-12 days intervals, corresponding to the flowering duration. Plants were grown in potting mix in a controlled environment (16-h day length, 21-24° C., 65-70% relative humidity).

The T₁ seeds produced can be collected and screened for transformants by germinating and growing seedlings at 22° C. with continuous light on a half-strength MS medium (pH 5.6) selection plate containing 1% (w/v) sucrose, 300 mg/L Timentin, and 25 mg/L DL-phosphinothricin to select for herbicide resistance. T₂ selfed seed populations can also be screened by immuno blot for the presence of the V5 eptiope.

T₂ selfed seeds may be analysed for oil content by GC. Approximately 50 individual transgenic lines (including control lines) may be selected for the next generation (10 plants/line) based on their oil content, or seed weight. T₂ plants may be grown and screened by PCR for copy number and identification of null sibing lines. T₂ seeds may be analysed in triplicate for oil content by NMR or GC/MS.

TABLE 5

| Original | | | | NUCLEIC ACID SEQ | |
|---|---|---|---|---|---|
| Source of | | | | OPTIMIZED FOR | EXPRESSED |
| DGAT | | | | CAMELINA | PROTEIN |
| Sequence | Name | Description | FIG. | SEQ ID NO: | SEQ ID: |
| T. majus | Tm [symbol] | DGAT1 from T. majus | 1, 2, 4, | 79 | 94 |
| Z. mays | ZmL | Long DGAT1 from Z. mays | 1, 2, 3, | 80 | 95 |
| Z. mays | ZmS | Short DGAT1 from Z. mays | 1, 2, 9 | 81 | 96 |
| Z. mays | ZmL [symbol] | Long DGAT1 from Z. mays with 1 [symbol] | 3, | 82 | 97 |
| | | and 3^{rd} regions of di-arginines | | | |
| | | disrupted | | | |
| Z. mays | ZmL [symbol] | Long DGAT1 from Z. mays with 2^{nd} | 3, | 83 | 98 |
| | | and 3^{rd} regions of | | | |
| | | di-arginines disrupted | | | |
| T. majus | Tm [symbol] | Truncated Tm disrupts AXXXA and | 4 | 84 | 99 |
| | | GXXXG | | | |
| T. majus | Tm [symbol] | Truncated Tm disrupts AXXXA and | 4 | 85 | 100 |
| | | GXXXG | | | |
| T. majus & Z. mays | Tm [symbol] ::ZmL | Disrupts AXXXA and GXXXG by | 6 | 86 | 101 |
| | | substitution | | | |
| Z. mays & T. majus | ZmS [symbol] Tm [symbol] | Disrupts AXXXA and GXXXG by | 6 | 87 | 102 |
| | | substitution | | | |
| T. majus & Z. mays | Tm [symbol] ::ZmL | Tm::ZmL chimera with disrupted di- | N/A | 88 | 103 |
| | | arginines | | | |
| Z. mays & T. majus | ZmS [symbol] ::Tm [symbol] | ZmS::Tm chimera with disrupted di- | N/A | 89 | 104 |
| | | arginines | | | |
| T. majus & Z. mays | Tm [symbol] ::ZmL | Tm::ZmL chimera with disrupted | 7 | 90 | 105 |
| | | AXXXA and additional di-arginines | | | |
| Z. mays & T. majus | ZmS [symbol] ::Tm [symbol] | ZmS::Tm chimera with disrupted | 8 | 91 | 106 |
| | | GXXXG and additional di-arginines | | | |
| Z. mays | ZmS [symbol] | ZmS with additional di-arginines | 9 | 92 | 107 |
| T. majus | Tm [symbol] | Tm with additional di-arginines | 9 | 93 | 108 |
| T. majus & Z. mays | Tm::ZmL | Chimera Tm N-terminus with ZmL C- | 5, 6, 7 | 109 | 111 |
| | | terminus (already in C. sativa) | | | |
| Z. mays & T. majus | ZmS::Tm [symbol] | Chimera ZmS N-terminus with Tm C- | 6 | 110 | 112 |
| | | terminus (already in C. sativa) | | | |

[symbol]

indicates data missing or illegible when filed

### Example 4: Evaluation of Plant DGAT1s with Modified Di-Arginine and AXXXA, GXXXG, AXXXG and GXXXA in Saccharomyces cerevisiae

The control DGAT1s At; Tm; ZmL; ΔN ZmL; Tm::ZmL and the DGAT1s with modified di-arginine and AXXXA, GXXXG, AXXXG and GXXXA Tm^{R25G,R26G,R27G}; Tm^{R25G,R26G,R27G}::ZmL; At^{A75S,G79S,G97S,G99S}; and At^{A75S,G79S,G93R,G94R,G95R,G97S,G99S} were over expressed in yeast and the TAG produced (as % DW) after 48 hours was determined (shown in Table 6).

TABLE 6

| Original | | | | | | | | g FA |
|---|---|---|---|---|---|---|---|---|
| Source | | | | | Cell | | | per L |
| of DGAT | | | | PROTEIN | DW | % FA | g FA | (% of |
| Sequence | Name | Description | FIG. | SEQ ID: | (g/L) | per cell | per L | control) |
| N/A | vector control | vector control | N/A | N/A | 5.36 | 3.22 | 0.173 | N/A |
| T. majus | Tm | DGAT1 from T. majus | 1, 2, 4, 9 | 69 | 5.79 | 8.36 | 0.495 | N/A |
| Z. mays | ZmL | Long DGAT1 from Z. mays | 1, 2, 3 | 70 | 7.26 | 12.6 | 0.915 | N/A |
| T. majus & | Tm::ZmL | Chimeric DGAT1 using Tm N- | 5, 6, 7 | 71 | 6.33 | 8.6 | 0.544 | 59.5 |
| Z. mays | | terminus and ZmL C-terminus | | | | | | (% of ZmL) |
| Z. mays | ΔN ZmL | ZmL with truncated | N/A | 72 | 6.67 | 12.7 | 0.847 | 92.6 |
| | | N-terminus | | | | | | (% of ZmL) |
| T. majus | Tm [symbol] | Tm Di-arginine motifs | N/A | 73 | 5.79 | 7.86 | 0.455 | 91.9 |
| | | substituted | | | | | | (% of Tm) |
| T. majus & | Tm [symbol] ::ZmL | Tm Di-arginine motifs | N/A | 74 | 5.33 | 7.3 | 0.389 | 71.5 |
| Z. mays | | substituted, and chimera | | | | | | (% of Tm::ZmL) |
| | | generated with ZmL | | | | | | |
| | | C-terminus | | | | | | |
| A. thaliana | At | DGAT1 from A. thaliana | 1, 2, 10, 11 | 75 | 5.46 | 6.43 | 0.351 | N/A |
| A. thaliana | At [symbol] | At with multiple disrupted | 10 | 76 | 5.31 | 6.68 | 0.355 | 101.1 |
| | | AXXXA and GXXXG | | | | | | (% of At) |
| A. thaliana | At [symbol] | At with maltipte disrupted | 11 | 77 | 5.89 | 6.72 | 0.396 | 112.8 |
| | | AXXXA and GXXXG and new RRRR | | | | | | (% of At) |
| A. thaliana | ΔN At | At with truncated | N/A | 78 | 6.02 | 8.68 | 0.523 | 147.3 |
| | | N-terminus | | | | | | (% of At) |

[symbol]

indicates data missing or illegible when filed

Microsomes were extracted from the yeast cells after 48 h culture. The extracts were subjected to PAGE-immunoblot (probing with either anti-V5 antibody or anti-Kar2 antibody). The most predominant band in the in-gel-stain-free image was scanned and quantified using BioRad's ChemiDoc software. Similarly, the immunofluorescence signals indicating the V5 tag of the DGAT1 and Kar2 marker protein of the ER were also scanned and quantified. The values and relative quantifications are shown in Table 7.

### Table 7.

TABLE 7

| | Signal Intensity | | | DGAT | DGAT |
|---|---|---|---|---|---|
| | Total | | DGAT1- | signal | signal |
| Protein | Membrane | Kar-2 | V5 | relative | relative |
| SEQ ID: | Protein | Signal | Signal | to MP | to Kar-2 |
| 75 | 13072992 | 8318706 | 1334200 | 0.102 | 0.160 |
| 76 | 12852240 | 6144000 | 1506348 | 0.117 | 0.245 |
| 77 | 13031997 | 6906966 | 1993800 | 0.153 | 0.289 |
| 69 | 13942980 | 15875928 | 5028670 | 0.361 | 0.317 |
| 73 | 16206056 | 11252126 | 1585332 | 0.098 | 0.141 |

Summary when Expressed in Yeast
Removal of di-arginine motifs in Tm decreased recombinant DGAT1 in yeast by approximately 56% (estimated by determining signal strength relative to Kar-2) and reduced FA production in yeast by approximately 10%.
The same perturbation of the N-terminal di-arginine motifs in TmZmL resulted in a decrease in FA production (g FA/L) in yeast cells of approximately 30%.
For the At DGAT1, disruption of AXXXA and GXXXG motifs and addition of a new RRR motif (near the C-terminus of the cytosolic variable N-terminus) increased recombinant DGAT1 in yeast microsomes by approximately 81% and increased FA production (g FA/L) in yeast cells by approximately 13%. In comparison disruption of AXXXA and GXXXG had little influence on FA production but increased accumulation of recombinant DGAT1 in yeast microsomes by approximately 53%.

### Example 5: Evaluation of Plant DGAT1s with Modified Di-Arginine and AXXXA, GXXXG, AXXXG and GXXXA in Camelina Sativa

The control DGAT1s (Tm^{S197A}, ZmL; ZmS; ZmS::Tm^{S170A}, Tm::ZmL) and the DGAT1s with modified di-arginine and AXXXA, GXXXG, AXXXG and GXXXA (ZmL^{R13G,R14S,R17S,R65G,R66G}; ZmL^{R33S,R34S,R37S,R65G,R66G}; Tm^{Δ68-94,S197A}; Tm^{Δ135-61,S197A}; Tm^{A64S,G80S}::ZmL; ZmS^{A10S,G17S,A36S,G37S}::Tm^{S170A}, Tm^{ΔR25,R26,R27}::ZmL; ZmS^{ΔR27,1.28,R29,R30}::Tm^{S170A}; Tm^{A64S,G78R,G79R,G80R}::ZmL; ZmS^{D12R,A14R,S44R,G45R,G46R}::Tm^{S170A}; Zm^{SA9R,A10R,S11R}; and Tm^{S6R,S7R,Q8R,S197A}) were over expressed in the seeds of Camelina sativa. The fatty acid content of the seed (as % DW) was determined and shown in Tables 8-13 (full data and statistics tables from each glasshouse). The data has been separated by glasshouse since the growing conditions varied within each glasshouse making comparisons between glasshouses inappropriate. However, an overall summary of the trends can be made; these are listed below.

SUMMARY when Expressed in Camelina
Disruption to ⅔ of the di-arginine clusters within the first 65 residues of ZmL led to a modest increase in seed FA content compared to ZmL 3.7 to 6.6% difference compared with ZmL)
First internal truncation of Tm led to decreases in seed FA content (−7.7% to −2.2%) compared to Tm^{S197A}
Second internal truncation of Tm led to decreases in seed FA content (−7.9% to −3.3% difference compared to Tm^{S197A}
Addition of di-arginine multi-motif within first 15 residues to ZmS, resulted in an increase in seed FA content (+12% compared to ZmS)
Addition of di-arginine multi-motif within first 15 residues to Tm^{S197A}, resulted in an increase in seed FA content (+15.5% compared to Tm^{S197A})
Disruption of first di-arginine motif in Tm::ZmL, resulted in a decrease in seed FA content (−21 to −7.5% compared to Tm::ZmL)
Disruption of first di-arginine motif in ZmS::Tm^{S170A}, resulted in a decrease in seed FA content (−15.6 to −14.5% compared to ZmS::Tm^{S170A})
Disrupt AXXXA and GXXXG in ZmS of ZmS::Tm^{S170A} resulted in a decrease in seed FA content (−8.0% compared to ZmS::Tm^{S170A})
Disrupt AXXXA and GXXXG in Tm of Tm::ZmL, resulted in an increase in seed FA content (+3.3% compared to Tm::ZmL)
Disrupt AXXXA and add di-arginine motif in Tm of Tm::ZmL, resulted in an increase in seed FA content (+6.3% compared to Tm::ZmL)
Disrupt AXXXA and add di-arginine motif in ZmS of ZmS::Tm^{S170A}, resulted in an increase in seed FA content (+0.2% compared to ZmS::Tm^{S170A})

TABLE 8
(GH3 SUMMARY)

| | | | | | TAG |
|---|---|---|---|---|---|
| | | | | | accumulation |
| | | | PROTEIN | Av % [symbol] A | compared to |
| DGAT1 Name | Description | FIG. | SEQ ID: | in seed | control |
| TmS197A | Control Tm DGAT | 1, 2, 4, | 94 | 26.32 | N/A |
| | | 9 | | | |
| ZmL | Control ZmL DGAT | 1, 2, 3 | 95 | 25.78 | N/A |
| ZmS | Control ZmS DGAT | 1, 2, 9 | 96 | 23.46 | N/A |
| ZmLR13G, R14S, R17S, R65G, R66G | disrupt di-arginines in ZmL | 3 | 97 | 25.74 ns | −0.2 (% of ZmL) |
| ZmLR33S, R34S, R37S, R65G, R66G | disrupi di-argiranes in ZmL | 3 | 98 | 27.49 ns | +6.6 (% of ZmL) |
| TmΔ68-94, S197A | internal [symbol] ation of Tm | 4 | 99 | 24.24 ns | −7.9 (% of Tm) |
| TmΔ35-61, S197A | internal [symbol] ation of Tm | 4 | 100 | 25.75 ns | −2.2 (% of Tm) |
| ZmSA9R, A10R, S11R | creates di-arginine in ZmS | 9 | 107 | 26.28 ns | +12.0 (% of ZmS) |

[symbol]

indicates data missing or illegible when filed

TABLE 9
(GH3 stats)

| | | DNA | Seed Size | | % Lipid | | Lipid | |
|---|---|---|---|---|---|---|---|---|
| | | Seq | (mg/seed) | Null Seed Size | (% DW) | Null % Lipid | (mg/seed) | Null Lipid |
| Line ID | DGAT1 | ID# | n = 6 | (p-Value) | n = 6 | (p-Value) | n = 6 | (p-Value) |
| WT | Wild-Type | | 1.22 | | 24.33 | | 0.30 | |
| pBR2 | Vector Control | | 1.16 | | 24.99 | | 0.29 | |
| Tm#2 | Tm | | 1.25 | 1.23 (0.6355) | 26.32 | 26.53 (0.6279) | 0.33 | 0.33 (0.8333) |
| ZmS#1 | ZmS | | 1.16 | 1.24 (0.0803) | 23.46 | 25.41 (0.3489) | 0.27 | 0.31 (0.2027) |
| ZmL#9 | ZmL | | 1.17 | 1.15 (0.6847) | 25.78 | 27.50 (0.3268) | 0.30 | 0.32 (0.6291) |
| 1-E | ZmL R13G R14S R17S R65G R65G | 82 | 1.21 | 1.17 (0.2047) | 27.09 | 26.58 (0.7733) | 0.33 | 0.31 (0.4663) |
| 1-D | | | 1.25 | 1.19 (0.1056) | 24.39 | 24.47 (0.9232) | 0.31 | 0.29 (0.2429) |
| 2-29 | ZmL R33S R34S R37S R65G R66 | 83 | 1.18 | 1.17 (0.7351) | 28.11 | 26.32 (0.1236) | 0.33 | 0.31 (0.2251) |
| 2-H11 | | | 1.19 | 1.15 (0.3879) | 24.91 | 25.22 (0.8865) | 0.30 | 0.29 (0.8381) |
| 2-H13 | | | 1.24 | 1.22 (0.5211) | 28.80 | 28.32 (0.7381) | 0.36 | 0.35 (0.5074) |
| 2-H14 | | | 1.28# | 1.23 (0.2203) | 28.12 | 28.15 (0.9815) | 0.36 | 0.35 (0.5713) |
| 3-A | Tm Delta 68-94 | 84 | 1.31 | 1.24 (0.1255) | 23.88 | 23.35 (0.7251) | 0.31 | 0.29 (0.3536) |
| 3-H | | | 1.27 | 1.22 (0.4819) | 24.59 | 23.68 (0.4413) | 0.31 | 0.29 (0.3886) |
| 4-48 | Tm Delta 35-61 | 85 | 1.27 | 1.25 (0.6464) | 24.70 | 23.93 (0.6589) | 0.31 | 0.30 (0.5273) |
| 4-50 | | | 1.25 | 1.22 (0.6047) | 26.80 | 24.67 (0.3536) | 0.34 | 0.30 (0.3494) |
| 11-6 | ZmS ASR A10R S11R | 92 | 1.27## | 1.15 (0.0003) | 25.93 | 26.02 (0.9319) | 0.33 | 0.30 (0.0402) |
| 11-7 | | | 1.25# | 1.21 (0.2503) | 27.10 | 26.81 (0.8333) | 0.34 | 0.32 (0.5236) |
| 11-13 | | | 1.29## | 1.18 (0.0019) | 25.58 | 23.36 (0.0899) | 0.33 | 0.28 (0.0163) |
| 11-15 | | | 1.25# | 1.19 (0.0335) | 24.26 | 25.25 (0.4124) | 0.31 | 0.30 (0.8027) |
| 11-H14 | | | 1.29# | 1.22 (0.1419) | 28.52 | 27.32 (0.6397) | 0.37# | 0.33 (0.3982) |

Glasshouse 3 Plants
(* = p-Value < 0.05, ** = p-value < 0.01, *** = p-value < 0.001, compared to WT and VC)
(# = p-value < 0.05, ## = p-value < 0.01, ### = p-value < 0.001, compared to the parent)

TABLE 10
(GH5 BATCH 1 SUMMARY)

| | | | PROTEIN | % FA in | TAG accumulation |
|---|---|---|---|---|---|
| DGAT1 Name | Description | FIG. | SEQ ID: | seed | compared to control |
| TmS197A | Control Tm DGAT | 1, 2, 4, 9 | 94 | 27.24 | N/A |
| ZmL | Control ZmL DGAT | 1, 2, 3 | 95 | 26.06 | N/A |
| ZmS | Control ZmS DGAT | 1, 2, 9 | 96 | 27.68 | N/A |
| ZmLR13G, R14S, R17S, | disrupt di-arginines in | 3 | 97 | 27.03 | +3.7 (% of ZmL) |
| R65G, R66G | ZmL | | | | |
| TmΔ68-94, S197A | internal truncation of Tm | 4 | 99 | 26.34 | −3.3 (% of Tm) |
| TmΔ35-61, S197A | internal truncation of Tm | 4 | 100 | 25.15 | −7.7 (% of Tm) |
| ZmS A10S, G17S, A36S, | disrupt AXXXA & GXXXG | 6 | 102 | 27.65 | −8.0 (% of ZmS::Tm) |
| G37S::TmS170A | | | | | |
| TmΔR25, R26, R27::ZmL | disrupt first di-arginine | 5 | 103 | 25.46 | −21.0 (% of Tm::ZmL) |
| | motif in Tm::ZmL | | | | |
| [symbol] mSΔ R17, L28, | disrupt first di-arginine | 5 | 104 | 25.36 | −15.6 (% of ZmS::Tm) |
| R29, R30::TmS170A | motif in ZmS::Tm | | | | |
| Tm::ZmL | Control Tm::ZmL Chimera | 6, 7 | 111 | 32.21 | N/A |
| ZmS::TmS170A | Control ZmS::Tm Chimera | 6 | 112 | 30.04 | N/A |

[symbol]

indicates data missing or illegible when filed

TABLE 11
(GH5 BATCH 1 STATS)

| | | DNA | Seed Size | Null | % Lipid | Null | Lipid | Null |
|---|---|---|---|---|---|---|---|---|
| | | Seq | [symbol] mg/seed [symbol] | Seed Size | [symbol] % DW [symbol] | % Lipid | [symbol] mg/seed [symbol] | Lipid |
| Line ID | DGAT1 | ID # | n = 6 | [symbol] p-Value [symbol] | n = 6 | [symbol] p-Value [symbol] | n = 6 | [symbol] p-Value [symbol] |
| WT | Wild type | | 1.24 | | 26.46 | | 0.33 | |
| pBR2 | Vector Control | | 1.18 | | 25.44 | | 0.30 | |
| TM [symbol] | Tm | | 1.38** | 1.29 (0.0056) | 27.24 | 26.48 (0.3299) | 0.38 | 0.34 (0.0099) |
| Zm [symbol] | ZmS | | 1.29 | 1.22 (0.1073) | 27.68 | 26.95 (0.2524) | 0.36 | 0.32 (0.0382) |
| Zm [symbol] | ZmL | | 1.20 | 1.17 (0.6170) | 26.06 | 26.47 (0.7288) | 0.31 | 0.31 (0.8847) |
| TM::Zm [symbol] | Tm::ZmL | | 1.16 | 1.20 (0.6159) | 31.71** | 27.42 (0.01025) | 0.36 | 0.33 (0.2458) |
| TM::Zm [symbol] | Tm::ZmL | | 1.25 | 1.16 (0.1419) | 32.71** | 26.37 (0.0029) | 0.41** | 0.31 (0.0057) |
| ZmS::Tm [symbol] | ZmS::Tm | | 1.42* | 1.23 (0.0325) | 30.04** | 26.09 (0.0006) | 0.43*** | 0.32 (0.0006) |
| 1-23 | ZmL R136 R145 R175 R65G | 82 | 1.29 | 1.17 (0.0274) | 27.03 | 26.65 (0.7625) | 0.35 | 0.31 (0.1463) |
| | R66G | | | | | | | |
| 3-10 | Tm Delta 68-94 | 84 | 1.15### | 1.19 (0.4990) | 25.73 | 25.92 (0.9449) | 0.30 | 0.31 (0.7562) |
| 3-17 | | | 1.21### | 1.05 (0.0126) | 26.95 | 22.85 (0.0883) | 0.33*# | 0.24 (0.0168) |
| 4- [symbol] | Tm Delta 35-61 | 85 | 1.27## | 1.14 (0.0159) | 25.15# | 25.20 (0.9724) | 0.32## | 0.28 (0.1746) |
| 5-1 | Tm Delta R25R26R27::ZmL | 88 | 1.16 | 1.21 (0.3930) | 25.52## | 25.78 (0.8661) | 0.30## | 0.31 (0.5845) |
| 5-2 | | | 1.32 | 1.22 (0.3007) | 25.92## | 26.69 (0.5484) | 0.34# | 0.33 (0.5523) |
| 5-11 | | | 1.28 | 1.33 (0.4643) | 24.94### | 26.01 (0.3461) | 0.32## | 0.35 (0.2959) |
| 6-14 | ZmS Delta R27L28R29R30::Tm | 89 | 1.33 | 1.12 (0.0038) | 24.48### | 20.59 (0.1847) | 0.33## | 0.24 (0.0506) |
| 6-15 | | | 1.29# | 1.25 (0.1860) | 26.23## | 23.89 (0.0366) | 0.34### | 0.30 (0.0117) |
| 8-14 | ZmS A10S G17S A36S G37S::Tm | 87 | 1.31*# | 1.20 (0.0703) | 30.63** | 26.01 (0.0010) | 0.40*** | 0.31 (0.0001) |
| 8- [symbol] | | | 1.34* | 1.15 (0.0022) | 24.66### | 25.93 (0.2769) | 0.33### | 0.30 (0.1115) |

Glasshouse 5-Batch 1 Plants
(*= p-Value < 0.05,
**=p-value <0.01,
***= p-value < 0.001, compared to WT and VC)
(#= p-value < 0.05,
##= p-value < 0.01, compared to the parent) [symbol]
indicates data missing or illegible when filed

TABLE 12
(GH7 SUMMARY)

| | | | | | TAG |
|---|---|---|---|---|---|
| | | | | | accumulation |
| | | | PROTEIN | % FA | compared to |
| DGAT1 Name | Description | FIG. | SEQ ID: | in seed | control |
| TmS197A | Control Tm DGAT | 1, 2, 4, | 94 | 25.08 | N/A |
| | | 9 | | | |
| ZmL | Control ZmL DGAT | 1, 2, 3 | 95 | 28.56 | N/A |
| ZmS | Control ZmS DGAT | 1, 2, 9 | 96 | 27.19 | N/A |
| TmA64S, G80S::ZmL | disrupt AXXXA & GXXXG | 6 | 101 | 30.59 | +3.3 (% of |
| | | | | | Tm::ZmL) |
| TmΔR25, R26, R27::ZmL | disrupt di-arginines in Tm portion | 5 | 103 | 27.40 | −7.5 (% of |
| | | | | | Tm::ZmL) |
| ZmSΔ R27, L28, R29, R30::TmS170A | disrupt di-arginines in ZmS::Tm | 5 | 104 | 27.83 | −14.5 (% of |
| | | | | | ZmS::Tm) |
| TmA64S, G78R, G79R, G80R::ZmL | disrupt AXXXA: added di-arginine in | 7 | 105 | 31.48 | +6.3 (% of |
| | Tm::ZmL | | | | Tm::ZmL) |
| ZmSD12R, A14R, S44R, G45R, G46R::TmS170A | disrupt AXXXA; added di-arginine in | 8 | 106 | 32.62 | +0.2 (% of |
| | ZmS::Tm | | | | ZmS::Tm) |
| TmS6R, S7R, Q8R, S197A | creates di-arginine in Tm | 9 | 108 | 28.97 | +15.5 (% of |
| | | | | | Tm) |
| Tm::ZmL | Control Tm::ZmL Chimera | 6, 7 | 111 | 29.62 | N/A |
| ZmS::TmS170A | Control ZmS::Tm Chimera | 6 | 112 | 32.54 | N/A |

TABLE 13
(GH7 STATS)

| | | DNA | Seed Size | Null | | % Lipid | | | Lipid | | |
|---|---|---|---|---|---|---|---|---|---|---|---|
| | | Seq | (mg/seed) | Seed Size | | (% DW) | Null % Lipid | | (mg/seed) | Null Lipid | |
| Line ID | DGAT1 | ID# | n = 6 | (p-Value) | | n = 6 | (p-Value) | | n = 6 | (p-Value) | |
| None | Wild-Type | | 1.17 | | | 27.16 | | | 0.32 | | |
| pBR2 | Vector Control | | 1.17 | | | 25.96 | | | 0.30 | | |
| Tm#2 | Tm | | 1.15 | 1.15 | (0.8877) | 25.08 | 23.91 | (0.2692) | 0.29 | 0.27 | (0.4350) |
| ZmS#1 | ZmS | | 1.16 | 1.14 | (0.7798) | 27.19 | 27.28 | (0.9549) | 0.31 | 0.31 | (0.8899) |
| ZmL#9 | ZmL | | 1.18 | 1.16 | (0.7606) | 28.56 | 26.17 | (0.1805) | 0.34 | 0.30 | (0.1409) |
| Tm::ZmL#5 | Tm::ZmL | | 1.21 | 1.20 | (0.7038) | 27.57 | 21.22 | (5.7E−01) | 0.33 | 0.25 | (5.1E−04) |
| Tm::ZmL#13 | Tm::ZmL | | 1.29** | 1.12 | (7.8E−04) | 31.66*** | 25.43 | (1.2E−04)) | 0.41*** | 0.29 | (9.0E−05) |
| ZmS::Tm#3 | ZmS::Tm | | 1.32** | 1.17 | (0.0222) | 32.93*** | 27.92 | (4.1E−04) | 0.44*** | 0.33 | (6.9E−04) |
| ZmS::Tm#4 | ZmS::Tm | | 1.46*** | 1.14 | (1.0E−05) | 32.14*** | 27.96 | (6.3E−04) | 0.47*** | 0.32 | (2.0E−05) |
| 5-15 | Tm Delta | 88 | 1.18## | 1.16 | (0.5842) | 28.10# | 26.87 | (0.3537) | 0.33## | 0.31 | (0.1856) |
| | R25R26R27::ZmL | | | | | | | | | | |
| 5-21 | | | 1.24 | 1.19 | (0.4706) | 26.70## | 25.30 | (0.4377) | 0.33## | 0.30 | (0.1811) |
| 6-23 | ZmS Delta | 89 | 1.20## | 1.18 | (0.5964) | 27.79## | 27.09 | (0.5961) | 0.33## | 0.32 | (0.5658) |
| | R27L28R29R30::Tm | | | | | | | | | | |
| 6-27 | | | 1.29## | 1.19 | (0.0609) | 27.86## | 25.34 | (0.0791) | 0.36*## | 0.30 | (0.0169) |
| 7 [symbol] | Tm A64S G80S::ZmL | 86 | 1.23 | 1.18 | (0.2181) | 30.13**# | 28.15 | (0.0255) | 0.37**# | 0.33 | (0.0419) |
| 7 [symbol] | | | 1.26* | 1.16 | (0.0537) | 30.35 | 27.34 | (0.1794) | 0.38* | 0.32 | (0.0289) |
| 7 [symbol] | | | 1.29 | 1.18 | (0.1179) | 32.42** | 26.93 | (0.0062) | 0.42*** | 0.32 | (1.3E−04) |
| 7 [symbol] | | | 1.32** | 1.21 | (0.0440) | 29.47 | 27.27 | (0.1677) | 0.39** | 0.33 | (0.0262) |
| 9-20 | Tm A64S G78R G79R | 90 | 1.15# | 1.16 | (0.8637) | 31.55** | 27.49 | (5.9E−04) | 0.36**# | 0.32 | (0.0113) |
| | G80R::ZmL | | | | | | | | | | |
| 9-21 | | | 1.14# | 1.18 | (0.1567) | 31.41* | 27.30 | (0.0323) | 0.35 | 0.32 | (0.1367) |
| 10-3 | ZmS D12R A14R S44R | 91 | 1.20## | 1.19 | (0.7306) | 31.28*** | 26.60 | (3.1E−04) | 0.38***## | 0.32 | (0.0170) |
| | G45R G45R::Tm | | | | | | | | | | |
| 10-13 | | | 1.22## | 1.21 | (0.8347) | 35.17** | 28.13 | (0.0016) | 0.43** | 0.34 | (0.0265) |
| 10-15 | | | 1.26## | 1.09 | (0.0354) | 31.41** | 27.44 | (0.0020) | 0.40**# | 0.30 | (0.0027) |
| 12-8 | Tm S6R S7R Q8R | 93 | 1.32**# | 1.16 | (0.0053) | 28.97# | 24.39 | (0.0151) | 0.38**## | 0.28 | (0.0013) |

Glasshouse 7 Plants
(*= p-Value < 0.05,
**= p-value < 0.01,
***= p-value < 0.001, compared to WT and VC)
(#=pvalue < 0.05,
##= p-value < 0.01, compared to the parent) [symbol]
indicates data missing or illegible when filed

### REFERENCES

Boulaflous A, Sait-Jore-Dupas C, Herranz-Gordo M-C, Pagny-Salehabadi S, Plasson C, Garidou F, Kiefer-Meyer M-C, Ritzenthaler C, Faye L, Gomord V (2009). BMC Plant Biology 9:144-166
Kleiger G, Grothe R, Mallick P, Eisenbert D (2002). Biochemistry 41:5990-5997
Michelsen K, Yuan H, Schwappach B (2005). EMBO reports 6:717-722
Parks G D, Lamb R A (1993). JBC 268:19101-19109
Scott R W, Winichayakul S, Roldan M, Cookson R, Willingham M, Castle M, Pueschel, R, Peng C-C, Tzen J T C, Roberts N J. (2010). Plant Biotechnology Journal. 8 (8): 912-927.
Shikano S, Li M (2003). PNAS 100:5783-5788
Teasdale R D, Jackson M R (1996). Annu. Rev. Cell Dev. Biol. 12:27-54
Teese M G, Langosh D (2015). Biochemistry 54:5125-5135
Xu J, Francis T, Mietkiewska E, Giblin E M, Barton D L, Zhang Y, Zhang M, Taylor D C (2008). Plant Biotech. J. 6:799-818

1. A method for producing a modified DGAT1 protein, the method comprising targeted manipulation of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
in the N-terminal region of the protein upstream of the acyl-CoA binding site of a DGAT1 protein, where R is arginine, A is alanine, G is glycine and X is any amino acid.

2. The method of claim 1 wherein the N-terminal region extends from the N-terminus of the DGAT1 protein to a position at least 1 amino acid upstream of the conserved motif ESPLSS (Glu-Ser-Pro-Leu-Ser-Ser) in the acyl-CoA binding site.

3. The method of claim 1 wherein the modified DGAT1 protein is at least 90% identical to the un-modified DGAT1 protein.

4. The method of claim 1 wherein the modified DGAT1 protein has a greater capacity to increase cellular lipid production than does the un-modified DGAT1 protein.

5. The method of claim 1 wherein when the modified DGAT1 protein is expressed in a cell, the cell produces more lipid than a suitable control cell in which modified protein is not expressed.

6. The method of claim 5 wherein when the modified DGAT1 protein is expressed in a cell, the cell produces at least 5% more than a suitable control cell in which modified protein is not expressed.

7. The method of claim 1 wherein the method includes a step of assessing the capacity of the modified DGAT1 protein to increase cellular lipid production relative to that of the un-modified DGAT1 protein.

8. The method of claim 1 wherein the method includes a step of selecting a modified DGAT1 protein with greater capacity to increase cellular lipid production than that of the un-modified DGAT1 protein.

9. The method of claim 1 wherein the modified DGAT1 protein is produced by expression from a polynucleotide encoding the modified DGAT1 protein.

10. The method of claim 9 wherein the modified DGAT1 protein is expressed in a cell or organism.

11. The method of claim 9 wherein the modified DGAT1 protein is expressed from a modified endogenous DGAT1 polynucleotide.

12. The method of claim 11 wherein the modified endogenous DGAT1 polynucleotide has been modified by a gene editing technology.

13. A modified DGAT1 protein, with an altered number or position of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
in the N-terminal region of the protein upstream of the acyl-CoA binding site of a DGAT1 protein, where R is arginine, A is Alanine, G is Glycine and X is any amino acid.

14. The modified DGAT1 protein of claim 13 wherein the N-terminal region extends from the N-terminus of the DGAT1 protein to a position at least 1 amino acid upstream of the conserved motif ESPLSS (Glu-Ser-Pro-Leu-Ser-Ser) in the acyl-CoA binding site.

15. The modified DGAT1 protein of claim 13 that is at least 90% identical to the un-modified DGAT1 protein.

16. The modified DGAT1 protein of claim 1 that has a greater capacity to increase cellular lipid production than does the un-modified DGAT1 protein.

17. The modified DGAT1 protein of claim 13 that is produced by targeted manipulation of at least one motif selected from:
a) a motif of the formula selected from RR, RXR, and RXXR,
b) a motif of the formula AXXXA,
c) a motif of the formula AXXXG,
d) a motif of the formula GXXXG, and
e) a motif of the formula GXXXA,
in the N-terminal region of the protein upstream of the acyl-CoA binding site of a DGAT1 protein, where R is arginine, A is alanine, G is glycine and X is any amino acid.

18. A polynucleotide encoding a modified DGAT1 of claim 13.

19. A construct comprising the polynucleotide of claim 18.

20. A cell comprising the modified DGAT1 protein of claim 13.

21. The cell of claim 20 that produces more lipid than does a suitable control cell.

22. The cell of claim 21 that produces at least 5% more lipid than a suitable control cell in which modified protein is not expressed.

23. A plant comprising the modified DGAT1 protein of claim 13 or a polynucleotide encoding the modified DGAT1 protein.

24. The plant of claim 23 in which the polynucleotide is an endogenous DGAT1 polynucleotide that has been modified in the plant to encode the modified DGAT1 protein.

25. The plant of claim 23 that produces more lipid, in at least one of its tissues or parts, than does the equivalent tissue or part in a suitable control plant.

26. The plant of claim 23 that produces at least 5% more lipid in at least one of its tissues or parts, than does a suitable control plant.

27. The plant of claim 23 that as a whole produces at least 5% more lipid than does a suitable control plant.

28. The part, propagule or progeny of a plant of claim 23 that comprises the modified DGAT1 protein or a polynucleotide encoding the modified DGAT1 protein.

29. The part, propagule or progeny of claim 28 that produces at least 5% more lipid than does an equivalent part, propagule or progeny of a suitable control plant.

30. An animal feedstock comprising the modified DGAT1 protein of claim 13 or a polynucleotide encoding the modified DGAT1 protein.

31. A biofuel feedstock comprising the modified DGAT1 protein of claim 13 or a polynucleotide encoding the modified DGAT1 protein.

32. A method for producing oil, the method comprising extracting lipid from the cell of claim 20 or a plant, plant part, propagule or progeny comprising the cell.

33. The method of claim 32 wherein the lipid is processed into at least one of:
a) a fuel,
b) an oleochemical,
c) a nutritional oil,
d) a cosmetic oil,
e) a polyunsaturated fatty acid (PUFA), and
f) a combination of any of a) to e).
